(19) United States
(12) Patent Application Publication
(10) Pub. No.: US 2013/0254944 A1
(43) Pub. Date: Sep. 26, 2013

(54) Herbicide Tolerant Soybeans and Methods of Use

(71) Applicants: PIONEER HI BRED INTERNATIONAL INC, JOHNSTON, IA (US); E.I. DU PONT DE NEMOURS AND COMPANY, WILMINGTON, DE (US)

(72) Inventors: JULIAN M. CHAKY, URBANDALE, IA (US); DAVID H. JOHNSTON, DES MOINES, IA (US); SCOTT A. SEBASTIAN, POLK CITY, IA (US); JOSHUA M. SHENDELMAN, ANKENY, IA (US); STEPHEN D. STRACHAN, OXFORD, PA (US); MARK VOGT, ANKENY, IA (US); KAY L. WALTER, SUNNYVALE, CA (US); JOHN B. WOODWARD, ANKENY, IA (US)

(73) Assignees: E.I. DU PONT DE NEMOURS AND COMPANY, WILMINGTON, DE (US); PIONEER HI BRED INTERNATIONAL INC, JOHNSTON, IA (US)

(21) Appl. No.: 13/795,885

(22) Filed: Mar. 12, 2013

Related U.S. Application Data
(60) Provisional application No. 61/613,703, filed on Mar. 21, 2012.

(51) Int. Cl. C12Q 1/68; C12N 15/82; A01N 43/50; A01C 1/00; A01N 47/36; A01N 47/38; A01N 43/90; A01H 5/10; A01N 43/54

(52) U.S. Cl. CPC C12Q 1/6895; A01H 5/10; C12N 15/8275; C12N 15/8277; C12N 15/8278; C12N 15/8274; A01N 43/50; A01N 43/54; A01N 47/36; A01N 47/38; A01N 43/90; A01C 1/00
USPC 800/300; 536/24.3; 435/6.11; 536/24.33; 504/253; 504/130; 504/243; 504/214; 504/215; 504/213; 504/212; 504/273; 504/241; 504/134; 504/136; 504/135; 504/139; 504/128; 111/100

(57) ABSTRACT

Soybean plants, germplasm, and seed comprising a mutant acetolactate synthase gene conferring improved herbicide tolerance, molecular markers useful for identifying and, optionally, selecting soybean plants displaying tolerance, improved tolerance, or susceptibility to a herbicide, and methods of their use are provided. Also provided are isolated polynucleotides, probes, kits, systems, and the like, useful for carrying out the methods described herein.

### REFERENCE TO SEQUENCE LISTING SUBMITTED ELECTRONICALLY

The official copy of the sequence listing is submitted electronically via EFS-Web as an ASCII formatted sequence listing with a file named “4270.seglist_ST25.txt” created on Feb. 28, 2013, and having a size of 45 kilobytes and is filed concurrently with the specification. The sequence listing contained in this ASCII formatted document is part of the specification and is herein incorporated by reference in its entirety.

### FIELD OF THE INVENTION

This invention relates to herbicide resistant soybean plants, molecular markers, and methods.

### BACKGROUND

Soybeans (Glycine max (L.) Merr.) are a major cash crop and investment commodity in North America and elsewhere. Soybean oil is one of the most widely used edible oils, and soybeans are used worldwide both in animal feed and in human food production. Additionally, soybean utilization is expanding to industrial, manufacturing, and pharmaceutical applications. Soybean varieties resistant to at least one herbicide provide efficient and effective weed control and crop management options, including treatment systems that take advantage of varying herbicide properties so that weed control could provide the best possible combination of flexibility and economy.

There is need for methods to identify and/or select soybean plants and germplasm with improved tolerance to ALS-inhibiting herbicides, including improved genetic markers for identifying plants possessing tolerance or susceptibility.

### SUMMARY

Soybean plants, germplasm and seed comprising a mutant acetolactate synthase gene conferring improved herbicide tolerance, molecular markers useful for identifying and, optionally, selecting soybean plants displaying tolerance, improved tolerance, or susceptibility to a herbicide, and methods of their use are provided. Also provided are isolated polynucleotides, probes, kits, systems, and the like, useful for carrying out the methods described herein.

### BRIEF DESCRIPTION OF DRAWINGS

FIG. 1 is a multiple sequence alignment of ALS1 polynucleotide sequences, the SNP variant is shown in bold font. The sequence alignment was generated using the PileUp program in GCG SeqWeb (Accelrys, San Diego, Calif., USA) using default settings of the pileupdna.cmp comparison table, the gap creation penalty=5, and the gap extension penalty=1.

FIG. 2 is a multiple sequence alignment of ALS2 polynucleotide sequences, the SNP is variant shown in bold font. The sequence alignment was generated using the PileUp program in GCG SeqWeb (Accelrys, San Diego, Calif., USA) using default settings of the pileupdna.cmp comparison table, the gap creation penalty=5, and the gap extension penalty=1.

FIG. 3 is a multiple sequence alignment of ALS1, ALS2, and HRA polypeptide sequences, the amino acid variants are shown in bold. The sequence alignment was generated using the PileUp program in GCG SeqWeb (Accelrys, San Diego, Calif., USA) using default settings of the BLOSSUM62 comparison scoring matrix, the gap creation penalty=8, and the gap extension penalty=2.

FIG. 4 provides an example of the dose response results of ALS1, ALS2, and ALS1+ALS2 soybean lines treated with an ALS-inhibiting herbicide and the isobole chart generated.

### SUMMARY OF THE SEQUENCES

SEQ ID NOs: 1-5, and 11-14 comprise nucleotide sequences of regions of the soybean genome, each capable of being used as a probe or primer, either alone or in combination, for the detection of marker locus S12761-1 on LG-C1, a marker locus comprising nucleotides 43645228-43645929 of Glyma04g37270.1, or any closely linked locus. In some examples, SEQ ID NO: 1 is used as a specific forward primer, and SEQ ID NO: 2 used as a specific reverse primer, with SEQ ID NOs: 3 and 4 used as allele specific probes. SEQ ID NO: 5 is a consensus sequence for Als1 (Glyma04g37270.1). SEQ ID NO: 5 comprises an amplicon produced by primer pair SEQ ID NOs: 1 and 2, and shows the location and sequence diversity of the SNP. SEQ ID NOs: 11-14 comprise wildtype and mutant ALS1 sequences. These sequences can be used as probes in various detection and characterization methodologies.

SEQ ID NOs: 6-10 comprise nucleotide sequences of regions of the soybean genome, each capable of being used as a probe or primer, either alone or in combination, for the detection of marker locus S12764-1 on LG-C2, a marker locus comprising nucleotides 14143182-14143881 of Glyma06g17790.1, or any closely linked locus. In some examples, SEQ ID NO: 6 is used as a specific forward primer, and SEQ ID NO: 7 used as a specific reverse primer, with SEQ ID NOs: 8 and 9 used as allele specific probes. SEQ ID NO: 10 is a consensus sequence for Als2 (Glyma06g17790.1). SEQ ID NO: 10 comprises an amplicon produced by primer pair SEQ ID NOs: 6 and 7 and shows the location and sequence diversity of the SNP. SEQ ID NOs: 15-18 comprise wildtype and mutant ALS2 sequences. These sequences can be used as probes in various detection and characterization methodologies.

SEQ ID NO: 19 is a polypeptide sequence of HRA.

SEQ ID NOs: 20-29 are oligonucleotide primers for the isolation of genomic soybean ALS polynucleotides.

SEQ ID NOs: 30-33 are oligonucleotide primers for synthesis of ALS1 and ALS2 cDNA polynucleotides.

SEQ ID NOs: 34-39 are RT-qPCR oligonucleotide primers for ALS1, ALS2, and EF1A polynucleotides.

### DETAILED DESCRIPTION

Methods for identifying a soybean plant or germplasm having tolerance, improved tolerance, or susceptibility to an ALS-inhibiting herbicide are provided, the methods comprising detecting at least one allele of one or more marker loci associated with ALS-inhibiting herbicide tolerance.

In some examples, the method involves identifying a soybean plant, germplasm or seed comprising an endogenous polynucleotide encoding a mutant acetolactate synthase gene in its genome, the method comprising isolating nucleic acids from the plant, germplasm or seed, and detecting at least one allele of one or more marker locus that is associated with herbicide resistance.

In some examples, the method involves detecting a single marker locus. In other examples, the method involves detecting two marker loci to provide a haplotype or marker profile for the plant or germplasm. In other examples, the method involves detecting two marker loci on different linkage groups or chromosomes to provide a marker profile for the plant or germplasm. In some examples, at least one marker locus is identified using methods of amplifying the marker locus or a portion thereof and detecting the marker amplicon produced.

In some examples, one or more marker locus is selected from the group consisting of S12761-1 on linkage group C1, a marker locus comprising nucleotides 43645228-43645929 of Glyma04g37270.1, a marker that detects a polymorphism at nucleotide 43645620 of Glyma04g37270.1, a marker that detects a polymorphism that encodes a P178S mutation in an acetolactate synthase gene on chromosome 4; S12764-1 on linkage group C2; a marker locus comprising nucleotides 14143182-14143881 of Glyma06g17790.1, a marker that detects a polymorphism at nucleotide 14143499 of Glyma06g17790.1, a marker that detects a polymorphism that encodes a W560L mutation in an acetolactate synthase gene on chromosome 6, a marker locus closely linked to any of the marker loci, and any combination of marker loci.

In some examples, at least one favorable allele is selected from the group consisting of allele T of S12761-1, allele T of S12764-1, allele T at nucleotide 43645620 of Glyma04g37270.1, allele Tat nucleotide 14143499 of Glyma06g17790.1, and any combination thereof. In some examples the method comprises detecting at least one favorable allele. In other examples, the method comprises detecting more than one favorable allele, up to and including all of the favorable alleles.

In some examples, the one or more alleles are favorable alleles that positively correlate with tolerance or improved tolerance to an ALS-inhibiting herbicide. In other examples, the one or more alleles are disfavored alleles that positively correlate with susceptibility or increased susceptibility to an ALS-inhibiting herbicide. In some examples, at least one allele is a favorable allele that positively correlates with improved herbicide resistance when compared to a soybean plant lacking the favorable allele.

In some examples, the ALS-inhibiting herbicide comprises a sulfonylurea herbicide. In some examples, sulfonylurea herbicides include but are not limited to amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flucetosulfuron, flupyrsulfuron-methyl (including sodium salt), foramsulfuron, halosulfuron-methyl, imazosulfuron, iodosulfuron-methyl (including sodium salt), mesosulfuron-methyl, metazosulfuron, metsulfuron-methyl, nicosulfuron, orthosulfamuron, oxasulfuron, primisulfuron-methyl, propyrisulfuron, prosulfuron, pyrazosulfuron-ethyl, rimsulfuron, sulfometuron-methyl, sulfosulfuron, thifensulfuron-methyl, triasulfuron, tribenuron-methyl, trifloxysulfuron (including sodium salt), triflusulfuron-methyl and tritosulfuron. In some examples, the sulfonylurea herbicide comprises a pyrimidinylsulfonylurea compound, a triazinylsulfonylurea compound, or any combination thereof. In some examples, the sulfonylurea herbicide comprises a chlorimuron, a sulfometuron, a thifensulfuron, a tribenuron or any combination thereof.

In some examples, the ALS-inhibiting herbicide comprises an imidazolinone herbicide. In some examples, the imidazolinone herbicide comprises an imazamethabenz, an imazamox, an imazapic, an imazapyr, an imazaquin, an imazethapyr, or any combination thereof. In some examples the ALS-inhibiting herbicide comprises a sulfonylaminocarbonyltriazolinone herbicide, also known as a triazolinone herbicide. In some examples the sulfonylaminocarbonyltriazolinone herbicide comprises a flucarbazone, a propoxycarbazone, a thiencarbazone-methyl or any combination thereof. In some examples the ALS-inhibiting herbicide comprises a triazolopyrimidine herbicide. In some examples the triazolopyrimidine herbicide comprises a cloransulam-methyl, a diclosulam, a florasulam, a flumetsulam, a metosulam, a penoxsulam, a pyrosulam, or any combination thereof. In some examples the ALS-inhibiting herbicide comprises a pyrimidinyl(thio)benzoate herbicide, also known as a pyrimidinyl(thio)ether herbicide. In some examples the pyrimidinyl(thio)benzoate herbicide comprises a bispyribac, a pyribenzoxim, a pyriftalid, pyrithiobac, a pyrminobac-methyl, and any combination thereof. In some examples, the ALS-inhibiting herbicide comprises a combination of two or more of an imidazolinone, a sulfonylurea, a sulfonylaminocarbonyltriazolinone, a triazolopyrimidine, a pyrimidinyl(thio)benzoate herbicide, or any combination thereof. In some examples, the ALS-inhibiting herbicide comprises an imazamethabenz, an imazamox, an imazapic, an imazapyr, an imazaquin, an imazethapyr, a pyrimidinylsulfonylurea compound, a triazinylsulfonylurea compound, a chlorimuron, a sulfometuron, a thifensulfuron, a tribenuron or any combination thereof.

In some examples, the ALS-inhibiting herbicide comprises a combination of ALS-inhibiting herbicides including combinations within any one of the five chemical subclasses (e.g., sulfonylureas, sulfonylaminocarbonyltriazolinones, triazolopyrimidines, pyrimidinyl(thio)benzoates and imidazolinones), as well as combinations across two or more of the five chemical subclasses. In some examples a combination of ALS-inhibiting herbicides may be applied as the combination, or may be applied separately. When applied separately, the herbicides may be applied concurrently, or sequentially. If applied sequentially, the herbicides may be provided at different plant growth stages, or at different points in the growing season.

In some examples, the ALS-inhibiting herbicide is used in combination with another herbicidal compound. In some examples, the other herbicide compound comprises one of more herbicides including but not limited to acetochlor, acifluorfen and its sodium salt, aclonifen, acrolein (2-propenal), alachlor, alloxydim, ametryn, amicarbazone, aminocyclopyrachor and its sodium or potassium salts or esters thereof, aminopyralid, amitrole, ammonium sulfamate, anilofos, asulam, atrazine, beflubutamid, benazolin, benazolin-ethyl, bencarbazone, benfluralin, benfuresate, bensulide, bentazone, benzobicyclon, benzofenap, bifenox, bilanafos, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorotoluron, chlorpropham, chlorthal-dimethyl, chlorthiamid, cinidon-ethyl, cinmethylin, clethodim, clodinafop-propargyl, clomazone, clomeprop, clopyralid, clopyralid-olamine, CUH-35 (2-methoxyethyl 2-[[[4-chloro-2-fluoro-5-[(1-methyl-2-propynyl)oxy]phenyl](3-fluoro-benzoyl)amino]carbonyl]-1-cyclohexene-1-carboxylate), cumyluron, cyanazine, cycloate, cycloxydim, cyhalofop-butyl, 2,4-D and any butotyl, butyl, isoctyl and isopropyl esters and any dimethylammonium, diolamine and trolamine salts, daimuron, dalapon, dalapon-sodium, dazomet, 2,4-DB and any dimethylammonium, potassium and sodium salts, desmedipham, desmetryn, dicamba and any diglycolammonium, dimethylammonium, potassium and sodium salts, dichlobenil, dichlorprop, diclofop-methyl, difenzoquat metilsulfate, diflufenican, diflufenzopyr, dimefuron, dimepiperate, dimethachlor, dimethametryn, dimethenamid, dimethenamid-P, dimethipin, dimethylarsinic acid and any sodium salt, dinitramine, dinoterb, diphenamid, diquat dibromide, dithiopyr, diuron, DNOC, endothal, EPTC, esprocarb, ethalfluralin, ethofumesate, ethoxyfen, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, fluazifop-butyl, fluazifop-P-butyl, flucarbazone, flucetosulfuron, fluchloralin, flufenacet, flufenpyr, flufenpyr-ethyl, flumiclorac-pentyl, flumioxazin, fluometuron, fluoroglycofen-ethyl, flurenol, flurenol-butyl, fluridone, fluorochloridone, fluoroxypyr, flurtamone, fluthiacet-methyl, fomesafen, fosamine-ammonium, glufosinate, glufosinate-ammonium, glyphosate and any salts such as ammonium, isopropylammonium, potassium, sodium (including sesquisodium) and trimesium (alternatively named sulfosate), haloxyfop-etotyl, haloxyfop-methyl, hexazinone, HOK-201 (N-(2,4-difluorophenyl)-1,5-dihydro-N-(1-methylethyl)-5-oxo-1-[(tetrahydro-2H-pyran-2-yl)-methyl]-4H-1,2,4-triazole-4-carboxamide), indanofan, indaziflam, iofensulfuron, ioxynil, ioxynil octanoate, ioxynil-sodium, ipfencarbazone, isoproturon, isouron, isoxaben, isoxaflutole, isoxachlortole, lactofen, lenacil, linuron, maleic hydrazide, MCPA and any salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesotrione, metam-sodium, metamifop, metamitron, metazachlor, methabenzthiazuron, methiozolin, methylarsonic acid and any calcium, monoammonium, monosodium and disodium salts, methyldymron, metobenzuron, metobromuron, metolachlor, S-metholachlor, metoxuron, metribuzin, molinate, monolinuron, naproanilide, napropamide, naptalam, neburon, norflurazon, orbencarb, oryzalin, oxadiargyl, oxadiazon, oxaziclomefone, oxyfluorfen, paraquat dichloride, pebulate, pelargonic acid, pendimethalin, pentanochlor, pentoxazone, perfluidone, pethoxyamid, phenmedipham, picloram, picloram-potassium, picolinafen, pinoxaden, piperofos, pretilachlor, prodiamine, profoxydim, prometon, prometryn, propachlor, propanil, propaquizafop, propazine, propham, propisochlor, propoxycarbazone, propyzamide, prosulfocarb, pyraclonil, pyraflufen-ethyl, pyrasulfotole, pyrazogyl, pyrazolynate, pyrazoxyfen, pyributicarb, pyridate, pyrimisulfan, pyrithiobac-sodium, quinclorac, quinmerac, quinoclamine, quizalofop-ethyl, quizalofop-P-ethyl, quizalofop-P-tefuryl, saflufenacil, sethoxydim, siduron, simazine, simetryn, sulcotrione, sulfentrazone, 2,3,6-TBA, TCA, TCA-sodium, tebutam, tebuthiuron, tefuryltrione, tembotrione, tepraloxydim, terbacil, terbumeton, terbuthylazine, terbutryn, thenylchlor, thiazopyr, thiencarbazone, thiobencarb, tiocarbazil, topramezone, tralkoxydim, tri-allate, triafamenone, triaziflam, triclopyr, triclopyr-butotyl, triclopyr-triethylammonium, tridiphane, trietazine, trifluralin, vernolate, and any combinations thereof.

Kits for characterizing a soybean plant, germplasm or seed are also provided. In some examples a kit comprises primers and/or probes for detecting one or more markers for one or more endogenous polynucleotides encoding a mutant acetolactate synthase associated with ALS-inhibiting herbicide tolerance, and instructions for using the primers and/or probes to detect the one or more marker loci and for correlating the detected marker loci with predicted tolerance to at least one herbicide. In some examples, one or more marker loci are selected from the group consisting of S12761-1, S12764-1, and markers closely linked thereto. In some examples, the primers or probes comprise one or more of SEQ ID NOs: 1-18. In some examples the kit further comprises a buffer or other reagent. In some examples, the kit can include one or more primers or probes for detecting one or more markers for another trait of interest. In some examples, the trait of interest is a transgene. In some example the trait of interest is a native trait.

Isolated polynucleotides are also provided. In one example, an isolated polynucleotide for detecting a marker locus is provided. In some examples isolated polynucleotides include S12761-1 on linkage group C1, a polynucleotide comprising nucleotides 43645228-43645929 of Glyma04g37270.1, a polynucleotide that detects a polymorphism at nucleotide 43645620 of Glyma04g37270.1, a polynucleotide that detects a polymorphism that encodes a P178S mutation in an acetolactate synthase gene on chromosome 4, S12764-1 on linkage group C2, a polynucleotide comprising nucleotides 14143182-14143881 of Glyma06g17790.1, a polynucleotide that detects a polymorphism at nucleotide 14143499 of Glyma06g17790.1; and, a polynucleotide that detects a polymorphism that encodes a W560L mutation in an acetolactate synthase gene on chromosome 6. In some examples, the polynucleotide comprises a nucleotide sequence selected from the group consisting of SEQ ID NOs: 1-18.

A soybean plant, germplasm, plant part, or seed comprising at least one endogenous polynucleotide encoding a mutant acetolactate synthase gene in its genome which confers improved herbicide resistance is provided. In some examples, the soybean plant, germplasm, plant part, or seed comprising at least one endogenous polynucleotide encoding a mutant acetolactate synthase gene in its genome which confers improved herbicide resistance is an elite soybean variety. In some examples said mutant acetolactate synthase gene encoding a mutation selected from the group consisting of a P178S mutation in an acetolactate synthase gene on chromosome 4, and a W560L mutation in an acetolactate synthase gene on chromosome 6, wherein said plant, germplasm, plant part, or seed has improved herbicide resistance when compared to a soybean plant or germplasm lacking a mutant acetolactate synthase gene in its genome. In some examples, the soybean plant or germplasm comprises a mutant acetolactate synthase gene encoding a P178S mutation in an acetolactate synthase gene on chromosome 4, and a W560L mutation in an acetolactate synthase gene on chromosome 6. In some examples, the soybean plant, germplasm, plant part, or seed has improved resistance to one or more ALS-inhibiting herbicides. In some examples, the soybean plant, germplasm, plant part, or seed comprising a mutant acetolactate synthase gene encoding a P178S mutation in an acetolactate synthase gene on chromosome 4, and a W560L mutation in an acetolactate synthase gene on chromosome 6 has improved resistance to one or more ALS-inhibiting herbicides that is synergistic, as compared to additive level of resistance of the individual mutations. In some examples the plant, germplasm, or seed having the two mutations have an improved resistance to one or more ALS-inhibiting herbicides that is at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 150%, 200%, or greater than 200% higher as compared to the additive levels of resistance provided by the single mutations. In some examples the plant, germplasm, or seed having the two mutations have an improved resistance to one or more ALS-inhibiting herbicides that is at least 1.2×, 1.3×, 1.4×, 1.5×, 1.6×, 1.7×, 1.8×, 1.9×, 2×, 2.25×, 2.5×. 2.75×, 3×, 3.25×, 3.5×, 3.75×, 4×, 4.5×, 5×, 6×, 7×, 8×, 9×, 10× or greater than 10× higher as compared to the additive levels of resistance provided by the single mutations. In some examples the plant, germplasm, or seed having the two mutations have an improved resistance to one or more ALS-inhibiting herbicides that provides resistance to a higher herbicide application rate that is 1.2×, 1.3×, 1.4×, 1.5×, 1.6×, 1.7×, 1.8×, 1.9×, 2×, 2.25×, 2.5×. 2.75×, 3×, 3.25×, 3.5×, 3.75×, 4×, 4.5×, 5×, 6×, 7×, 8×, 9×, 10× or greater than 10× higher as compared to herbicide application rate corresponding to the additive levels of resistance provided by the single mutations. In some examples the plant, germplasm, or seed having the two mutations have an improved resistance to one or more ALS-inhibiting herbicides that such that the damage or toxicity is reduced at least at least 5%, 10%, 15%, 20%, 25%, 30%, 35%, 40%, 45%, 50%, 55%, 60%, 65%, 70%, 75%, 80%, 85%, 90%, 95%, or greater than 95% as compared to the damage or toxicity from additive levels of resistance provided by the single mutations. In some examples, the soybean plant, germplasm, plant part, or seed has improved resistance to one or more ALS-inhibiting herbicides including a sulfonylurea herbicide, a sulfonylaminocarbonyltriazolinone, a triazolopyrimidines, pyrimidinyl(thio)benzoates, and an imidazolinone herbicide. In some examples, the sulfonylurea herbicide comprises a pyrimidinylsulfonylurea or a triazinylsulfonylurea. In some examples, the sulfonylurea herbicide comprises a chlorimuron, a sulfometuron, a thifensulfuron, or a tribenuron. In some examples, the soybean plant, germplasm, plant part, or seed further comprises resistance to a herbicidal formulation comprising a compound selected from the group consisting of a hydroxyphenylpyruvatedioxygenase inhibitor, a phosphanoglycine (including but not limited to a glyphosate), a sulfonamide, an imidazolinone, a bialaphos, a phosphinothricin, a metribuzin, a mesotrione, an isoxaflutole, an azafenidin, a butafenacil, a sulfosate, a glufosinate, a dicamba, a 2,4-D, and a protox inhibitor. In some examples, resistance to the herbicidal formulation is conferred by a transgene. In some examples, the plant or germplasm further comprises a trait selected from the group consisting of drought tolerance, stress tolerance, disease resistance, herbicide resistance, enhanced yield, modified oil, modified protein, tolerance to chlorotic conditions, and insect resistance, or any combination thereof. In some examples, the trait is selected from the group consisting of brown stem rot resistance, charcoal rot drought complex resistance, Fusarium resistance, Phytophthora resistance, stem canker resistance, sudden death syndrome resistance, Sclerotinia resistance, Cercospora resistance, anthracnose resistance, target spot resistance, frogeye leaf spot resistance, soybean cyst nematode resistance, root knot nematode resistance, rust resistance, high oleic content, low linolenic content, aphid resistance, stink bug resistance, and iron chlorosis deficiency tolerance, or any combination thereof. In some examples, one or more of the traits is conferred by one or more transgenes, by one or more native loci, or any combination thereof.

In another example a method of producing a cleaned soybean seed is provided, the method comprising cleaning a soybean seed comprising at least one endogenous polynucleotide encoding a mutant acetolactate synthase gene in its genome which confers improved herbicide resistance is provided. In some examples said mutant acetolactate synthase gene encode a mutation selected from the group consisting of a P178S mutation in an acetolactate synthase gene on chromosome 4, and a W560L mutation in an acetolactate synthase gene on chromosome 6, wherein said seed or plant produced therefrom has improved herbicide resistance when compared to a soybean plant or germplasm lacking a mutant acetolactate synthase gene in its genome. In some examples, the seed or plant produced therefrom comprises a mutant acetolactate synthase gene encoding a P178S mutation in an acetolactate synthase gene on chromosome 4, and a W560L mutation in an acetolactate synthase gene on chromosome 6. In some examples, the cleaned soybean seed has enhanced yield characteristics when compared to a soybean seed which has not been cleaned. Cleaned soybean seed produced by the methods are also provided.

In another example a method of producing a treated soybean seed is provided, the method comprising treating a soybean seed comprising at least one endogenous polynucleotide encoding a mutant acetolactate synthase gene in its genome which confers improved herbicide resistance is provided. In some examples said mutant acetolactate synthase gene encodes a mutation selected from the group consisting of a P178S mutation in an acetolactate synthase gene on chromosome 4, and a W560L mutation in an acetolactate synthase gene on chromosome 6, wherein said seed or plant produced therefrom has improved herbicide resistance when compared to a soybean plant or germplasm lacking a mutant acetolactate synthase gene in its genome. In some examples, the seed or plant produced therefrom comprises a mutant acetolactate synthase gene encoding a P178S mutation in an acetolactate synthase gene on chromosome 4, and a W560L mutation in an acetolactate synthase gene on chromosome 6. In some examples, the seed treatment comprises a fungicide, an insecticide, or any combination thereof. In some examples the seed treatment comprises trifloxystrobin, metalaxyl, imidacloprid, Bacillus spp., and any combination thereof. In some examples the seed treatment comprises picoxystrobin, penthiopyrad, cyantraniliprole, chlorantraniliprole, and any combination thereof. In some examples, the seed treatment improves seed germination under normal and/or stress environments, early stand count, vigor, yield, root formation, nodulation, and any combination thereof when compared to a soybean seed which has not been treated. In some examples seed treatment reduces seed dust levels, insect damage, pathogen establishment and/or damage, plant virus infection and/or damage, and any combination thereof. Treated soybean seed produced by the methods are also provided.

In some examples, methods include a method of producing an herbicide resistant soybean crop, said method comprising planting the seed of the invention. In some examples, the method further comprises applying at least one herbicide in an amount sufficient to differentiate between resistant plants and susceptible plants. In some examples, the herbicide comprises a sulfonylurea herbicide. In some examples the sulfonylurea herbicide comprises a pyrimidinylsulfonylurea compound or a triazinylsulfonylurea compound. In some examples the sulfonylurea herbicide comprises a chlorimuron, a sulfometuron, a thifensulfuron, or a tribenuron. In some examples, the herbicide is applied as a pre-emergent herbicide. In some examples, the herbicide is applied as a post-emergent herbicide. In some examples, the method further comprises applying to the crop and/or weeds in the field a simultaneous or a chronologically staggers application of the herbicide, and optionally an additional herbicide formulation. In examples including application of an additional herbicide formulation, the additional herbicide formulation can comprise an active ingredient including but not limited to a hydroxyphenylpyruvatedioxygenase inhibitor, a phosphanoglycine (including but not limited to a glyphosate), a sulfonamide, an imidazolinone, a bialaphos, a phosphinothricin, a mesotrione, an isoxaflutole, an azafenidin, a butafenacil, a sulfosate, a glufosinate, a dicamba, a 2,4-D, a metribuzin, a protox inhibitor, or any combinations thereof. In some examples, the crop seeds or plants further comprise tolerance to the active ingredient of the additional herbicide formulation. In some examples, tolerance to the active ingredient of the additional herbicide formulation is provided by a transgene which confers the tolerance.

In some examples, methods include a method for selectively controlling weeds in a field containing a crop comprising planting a field with crop seeds or plants comprising at least one favorable allele of a marker locus of the invention, wherein the seeds or plants have tolerance to a herbicide; and, applying to the crop and/or weeds in the field a sufficient amount of the herbicide to control the weeds without significantly affecting the crop. In some examples, the herbicide comprises a sulfonylurea herbicide. In some examples the sulfonylurea herbicide comprises a pyrimidinylsulfonylurea compound or a triazinylsulfonylurea compound. In some examples the sulfonylurea herbicide comprises a chlorimuron, a sulfometuron, a thifensulfuron, or a tribenuron. In some examples, the herbicide is applied as a pre-emergent herbicide. In some examples, the herbicide is applied as a post-emergent herbicide. In some examples, the method further comprises applying to the crop and weeds in the field a simultaneous or a chronologically staggered application of the herbicide, and optionally an additional herbicide formulation. In examples including application of an additional herbicide formulation, the additional herbicide formulation can comprise an active ingredient including but not limited to a hydroxyphenylpyruvatedioxygenase inhibitor, a phosphanoglycine (including but not limited to a glyphosate), a sulfonamide, an imidazolinone, a bialaphos, a phosphinothricin, a mesotrione, an isoxaflutole, an azafenidin, a butafenacil, a sulfosate, a glufosinate, a dicamba, a 2,4-D, a metribuzin, a protox inhibitor, or any combinations thereof. In some examples, the crop seeds or plants further comprise tolerance to the active ingredient of the additional herbicide formulation. In some examples, tolerance to the active ingredient of the additional herbicide formulation is provided by a transgene which confers the tolerance.

Also provided is a method to determine an herbicide application rate which can be used to differentiate between soybean plants or seeds comprising at least one endogenous polynucleotide encoding a mutant acetolactate synthase gene in its genome which confers improved herbicide resistance to an ALS-inhibiting herbicide, the method comprising providing a soybean seed, determining a marker profile for the soybean seed, wherein determining the marker profile comprises use of one or more markers of S12761-1 on linkage group C1, a marker locus comprising nucleotides 43645228-43645929 of Glyma04g37270.1, a marker that detects a polymorphism at nucleotide 43645620 of Glyma04g37270.1, a marker that detects a polymorphism that encodes a P178S mutation in an acetolactate synthase gene on chromosome 4; S12764-1 on linkage group C2, a marker locus comprising nucleotides 14143182-14143881 of Glyma06g17790.1, a marker that detects a polymorphism at nucleotide 14143499 of Glyma06g17790.1, a marker that detects a polymorphism that encodes a W560L mutation in an acetolactate synthase gene on chromosome 6, a marker locus closely linked to any of the marker loci, and any combination thereof, and using the marker profile to determine an application rate for at least one ALS-inhibiting herbicide that controls any susceptible or less tolerant plants. In some examples the application rate is a rate which controls any susceptible or less tolerant plants without significantly affecting plants comprising at least one endogenous polynucleotide encoding a mutant acetolactate synthase gene in its genome. In some examples, the profile is determined from a sample from the seed, for example a seed chip. In some examples, the seed is planted and allowed to germinate, and the profile is determined using a sample from the germinated plant, such as a leaf tissue sample. In some examples, the method further comprises applying at least one ALS-inhibiting herbicide. In some examples, the method further comprises determining the plant or seed response to the applied herbicide to validate the predicted application rate. In some examples, the method further comprises determining an herbicide application rate which can differentiate between soybean plants or seeds which are heterozygous for Als1, homozygous for Als1 or als1, heterozygous for Als2, homozygous for Als2 or als2, and any combination thereof. In some examples, the ALS-inhibiting herbicide comprises a sulfonylurea herbicide. In some examples, the sulfonylurea herbicide comprises a pyrimidinylsulfonylurea compound or a triazinylsulfonylurea compound. In some examples, the sulfonylurea herbicide comprises a chlorimuron, a sulfometuron, a thifensulfuron, a tribenuron, or any combination thereof. In some examples, the ALS-inhibiting herbicide comprises an imidazolinone herbicide. In some examples, the imidazolinone herbicide comprises an imazamethabenz, an imazamox, an imazapic, an imazapyr, an imazaquin, an imazethapyr, or any combination thereof. In some examples, the ALS-inhibiting herbicide comprises a combination of an imidazolinone herbicide and a sulfonylurea herbicide. In some examples, the ALS-inhibiting herbicide comprises an imazamethabenz, an imazamox, an imazapic, an imazapyr, an imazaquin, an imazethapyr, a pyrimidinylsulfonylurea compound, a triazinylsulfonylurea compound, a chlorimuron, a sulfometuron, a thifensulfuron, a tribenuron or any combination thereof. In some examples said mutant acetolactate synthase gene encodes a mutation selected from the group consisting of a P178S mutation in an acetolactate synthase gene on chromosome 4, and a W560L mutation in an acetolactate synthase gene on chromosome 6, wherein said seed or plant produced therefrom has improved herbicide resistance when compared to a soybean plant or germplasm lacking a mutant acetolactate synthase gene in its genome. In some examples, the seed or plant produced therefrom comprises a mutant acetolactate synthase gene encoding a P178S mutation in an acetolactate synthase gene on chromosome 4, and a W560L mutation in an acetolactate synthase gene on chromosome 6

A method to produce a soybean crop tolerant to higher ALS-inhibiting herbicide application rates is provided, said method comprises planting soybean seed comprising at least one endogenous polynucleotide encoding a mutant acetolactate synthase gene in its genome which confers improved herbicide resistance to an ALS-inhibiting herbicide in a field; and, applying a herbicidal compound comprising at least one ALS-inhibitor to the field in a sufficient amount of the herbicide to control any susceptible and less tolerant plants without significantly affecting the crop, wherein the herbicidal compound is applied in an amount greater than the amount used on a soybean crop lacking the endogenous polynucleotide encoding a mutant acetolactate synthase gene. In some examples, the herbicide comprises at least one ALS-inhibiting in an amount greater than 0.5× of the recommended field rate. In some examples, the seed comprises the P178S mutation in an acetolactate synthase gene on chromosome 4 and the W560L mutation in an acetolactate synthase gene on chromosome 6, and wherein the herbicide comprises at least one ALS-inhibiting in an amount greater than 1.5× of the recommended field rate.

A method of producing a soybean crop in a field comprising at least one residual ALS-inhibiting herbicide is provided, said method comprising planting soybean seed comprising at least one endogenous polynucleotide encoding a mutant acetolactate synthase gene in its genome which confers improved herbicide resistance to an ALS-inhibiting herbicide in the field having residual ALS-inhibiting herbicide present.

In certain examples, detecting comprises amplifying the marker locus or a portion of the marker locus and detecting the resulting amplified marker amplicon. In particular examples, the amplifying comprises: 1) admixing an amplification primer or amplification primer pair and, optionally at least one nucleic acid probe, with a nucleic acid isolated from the first soybean plant or germplasm, wherein the primer or primer pair and optional probe is complementary or partially complementary to at least a portion of the marker locus and is capable of initiating DNA polymerization by a DNA polymerase using the soybean nucleic acid as a template; and 2) extending the primer or primer pair in a DNA polymerization reaction comprising a DNA polymerase and a template nucleic acid to generate at least one amplicon. In particular examples, the detection comprises real time PCR analysis.

The methods can be used to aid in the selection of breeding plants, lines, and populations containing tolerance to an ALS-inhibiting herbicide for use in introgression of this trait into elite soybean germplasm, or germplasm of proven genetic superiority suitable for variety release. Also provided is a method for introgressing a soybean QTL, marker, marker profile, and/or haplotype associated with an ALS-inhibiting herbicide tolerance into non-tolerant or less tolerant soybean germplasm. According to the method, markers, marker profiles, and/or haplotypes are used to select soybean plants containing the improved tolerance trait. Plants so selected can be used in a soybean breeding program. Through the process of introgression, the QTL, marker, marker profile, and/or haplotype associated with an improved ALS-inhibiting herbicide tolerance is introduced from plants identified using marker-assisted selection (MAS) to other plants. According to the method, agronomically desirable plants and seeds can be produced containing the QTL, marker, marker profile, and/or haplotype associated with an ALS-inhibiting herbicide tolerance from germplasm containing the QTL, marker, marker profile, and/or haplotype.

Also provided herein is a method for producing a soybean plant adapted for conferring improved ALS-inhibiting herbicide tolerance. First, donor soybean plants for a parental line containing one or more tolerance QTL, marker, haplotype, and/or marker profile are selected. According to the method, selection can be accomplished via MAS as explained herein. Selected plant material may represent, among others, an inbred line, a hybrid line, a heterogeneous population of soybean plants, or an individual plant. According to techniques well known in the art of plant breeding, this donor parental line is crossed with a second parental line. In some examples, the second parental line is a high yielding line. This cross produces a segregating plant population composed of genetically heterogeneous plants. Plants of the segregating plant population are screened for one or more of the tolerance QTL, marker, haplotype, and/or marker profile. Further breeding may include, among other techniques, additional crosses with other lines, with hybrids, backcrossing, or self-crossing. The result is a line of soybean plants that has improved tolerance to one or more ALS-inhibiting herbicide and optionally also has other desirable traits from one or more other soybean lines.

Soybean plants, germplasm, seeds, tissue cultures, variants and mutants having improved ALS-inhibiting herbicide tolerance produced by the foregoing methods are provided. Also provided are isolated nucleic acids, kits, and systems useful for the identification and selection methods disclosed herein.

It is to be understood that this invention is not limited to particular embodiments, which can, of course, vary. It is also to be understood that the terminology used herein is for the purpose of describing particular embodiments only, and is not intended to be limiting. Further, all publications referred to herein are incorporated by reference for the purpose cited to the same extent as if each was specifically and individually indicated to be incorporated by reference herein.

### DEFINITIONS

As used in this specification and the appended claims, terms in the singular and the singular forms “a,” “an,” and “the,” for example, include plural referents unless the content clearly dictates otherwise. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. In a claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole. The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention.

Certain definitions used in the specification and claims are provided below. In order to provide a clear and consistent understanding of the specification and claims, including the scope to be given such terms, the following definitions are provided:

“ALS” is a common abbreviation for the enzyme acetolactate synthase. This enzyme is also known as acetohydroxy acid synthase (AHAS). As used herein “ALS” indicates a polynucleotide or polypeptide encoding acetolactate synthase enzyme activity, or a sequence derived therefrom. “ALS1” and “ALS2” are each used to denote a genetic locus comprising a polynucleotide that codes for acetolactate synthase enzyme activity. “Als1” and “als1” are used to denote the resistant and susceptible (wildtype) alleles respectively at the ALS1 locus in soybean. “Als2” and “als2” are used to denote the resistant and susceptible (wildtype) alleles respectively at the ALS2 locus in soybean.

“ALS-inhibiting herbicide” refers to a chemical compound causing herbicidal effects such as growth reduction through inhibition of the plant enzyme acetolactate synthase (ALS), also known as acetohydroxy acid synthase (AHAS) (EC 2.2.1.6).

“Allele” means any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant. A favorable allele is an allele correlated with the preferred phenotype. A favorable allele is typically denoted as a nucleotide variant on one strand at a specified position of a polynucleotide, but clearly includes the nucleotide at the corresponding position on the complementary strand of the polynucleotide. For example, a favorable allele “T” at position 10 of polynucleotide X includes the “A” at the corresponding position of the other strand of polynucleotide X based nucleotide base pairing.

The term “amplifying” in the context of nucleic acid amplification is any process whereby additional copies of a selected nucleic acid (or a transcribed form thereof) are produced. An “amplicon” is an amplified nucleic acid, e.g., a nucleic acid that is produced by amplifying a template nucleic acid by any available amplification method.

“Backcrossing” is a process in which a breeder crosses a progeny variety back to one of the parental genotypes one or more times.

The term “chromosome segment” designates a contiguous linear span of genomic DNA that resides in planta on a single chromosome. “Chromosome interval” refers to a chromosome segment defined by specific flanking marker loci.

“Cultivar” and “variety” are used synonymously and mean a group of plants within a species (e.g., Glycine max) that share certain genetic traits that separate them from other possible varieties within that species. Soybean cultivars are inbred lines produced after several generations of self-pollinations. Individuals within a soybean cultivar are homogeneous, nearly genetically identical, with most loci in the homozygous state.

An “elite line” is an agronomically superior line that has resulted from many cycles of breeding and selection for superior agronomic performance. Numerous elite lines are available and known to those of skill in the art of soybean breeding.

An “elite population” is an assortment of elite individuals or lines that can be used to represent the state of the art in terms of agronomically superior genotypes of a given crop species, such as soybean.

An “exotic soybean strain” or an “exotic soybean germplasm” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

A “genetic map” is a description of genetic linkage relationships among loci on one or more chromosomes or linkage groups within a given species, generally depicted in a diagrammatic or tabular form.

“Genotype” refers to the genetic constitution of a cell or organism.

“Germplasm” means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

“Herbicide resistance” and “herbicide tolerance” are used interchangeably to classify plants that when treated with a particular herbicide will show reduced damage or symptoms as compared to an appropriate control plant treated under substantially identical conditions.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

“Introgression” means the entry or introduction of a gene, a transgene, a QTL, a marker, a haplotype, a marker profile, a trait, a trait locus, or a chromosomal segment from the genome of one plant into the genome of another plant.

The terms “label” and “detectable label” refer to a molecule capable of detection. A detectable label can also include a combination of a reporter and a quencher, such as are employed in FRET probes or TaqMan™ probes. The term “reporter” refers to a substance or a portion thereof which is capable of exhibiting a detectable signal, which signal can be suppressed by a quencher. The detectable signal of the reporter is, e.g., fluorescence in the detectable range. The term “quencher” refers to a substance or portion thereof which is capable of suppressing, reducing, inhibiting, etc., the detectable signal produced by the reporter. As used herein, the terms “quenching” and “fluorescence energy transfer” refer to the process whereby, when a reporter and a quencher are in close proximity, and the reporter is excited by an energy source, a substantial portion of the energy of the excited state nonradiatively transfers to the quencher where it either dissipates nonradiatively or is emitted at a different emission wavelength than that of the reporter.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor. Traditionally, a subline has been derived by inbreeding the seed from an individual soybean plant selected at the F3 to F5 generation until the residual segregating loci are homozygous (fixed) across most or all loci. Commercial soybean varieties (or lines) are typically produced by aggregating (bulking) the self-pollinated progeny of a single F3 to F5 plant from a controlled cross between 2 genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. Marker-based sublines that differ from each other based on qualitative polymorphism at the DNA level at one or more specific marker loci are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (e.g., yield, tolerance, etc.).

“Linkage” refers to a phenomenon wherein alleles on the same chromosome tend to segregate together more often than expected by chance if their transmission was independent. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a map distance of 1.0 centiMorgan (1.0 cM).

With regard to physical position on a chromosome, closely linked markers can be separated, for example, by about 1 megabase (Mb; 1 million nucleotides), about 500 kilobases (Kb; 1000 nucleotides), about 400 Kb, about 300 Kb, about 200 Kb, about 100 Kb, about 50 Kb, about 25 Kb, about 10 Kb, about 5 Kb, about 4 Kb, about 3 Kb, about 2 Kb, about 1 Kb, about 500 nucleotides, about 250 nucleotides, or less.

When referring to the relationship between two genetic elements, such as a genetic element contributing to tolerance and a proximal marker, “coupling” phase linkage indicates the state where the “favorable” allele at the tolerance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for tolerance) is physically linked with an “unfavorable” allele at the proximal marker locus, and the two “favorable” alleles are not inherited together (i.e., the two loci are “out of phase” with each other).

“Linkage disequilibrium” refers to a phenomenon wherein alleles tend to remain together in linkage groups when segregating from parents to offspring, with a greater frequency than expected from their individual frequencies.

“Linkage group” refers to traits or markers that generally co-segregate. A linkage group generally corresponds to a chromosomal region containing genetic material that encodes the traits or markers.

“Locus” is a defined segment of DNA.

A “map location,” a “map position,” or, “relative map position” is an assigned location on a genetic map relative to linked genetic markers where a specified marker can be found within a given species. Map positions are generally provided in centimorgans. A “physical position” or “physical location” is the position, typically in nucleotide bases, of a particular nucleotide, such as a SNP nucleotide, on the chromosome.

“Mapping” is the process of defining the linkage relationships of loci through the use of genetic markers, populations segregating for the markers, and standard genetic principles of recombination frequency.

“Marker” or “molecular marker” is a term used to denote a nucleic acid or amino acid sequence that is sufficiently unique to characterize a specific locus on the genome. Any detectible polymorphic trait can be used as a marker so long as it is inherited differentially and exhibits linkage disequilibrium with a phenotypic trait of interest.

“Marker assisted selection” refers to the process of selecting a desired trait or traits in a plant or plants by detecting one or more nucleic acids from the plant, where the nucleic acid is linked to the desired trait, and then selecting the plant or germplasm possessing those one or more nucleic acids.

“Marker profile” denotes a combination of particular alleles present within a particular plant's genome at two or more marker loci which are not linked, including but not limited to instances when two or more loci are on two or more different linkage groups.

“Haplotype” refers to a combination of particular alleles present within a particular plant's genome at two or more linked marker loci, for instance at two or more loci on a particular linkage group. A haplotype can include 2, 3, 4, 5, 6, 7, 8, 9, 10, 11, 12, 13, 14, 15, 16, 17, 18, 19, 20, or more linked marker loci used to define a haplotype for a particular plant.

“Mode of action” generally refers to the metabolic or physiological process within the plant that the herbicide inhibits or otherwise impairs, whereas “site of action” generally refers to the physical location or biochemical site within the plant where the herbicide acts or directly interacts.

The term “plant” includes reference to an immature or mature whole plant, including a plant from which seed or grain or anthers have been removed. Seed or an embryo that will produce the plant is also considered to be the plant.

“Plant parts” means any portion or piece of a plant, including leaves, stems, buds, roots, root tips, anthers, seed, grain, embryo, pollen, ovules, flowers, cotyledons, hypocotyls, pods, flowers, shoots, stalks, tissues, tissue cultures, cells, and the like.

“Polymorphism” means a change or difference between two related nucleic acids. A “nucleotide polymorphism” refers to a nucleotide that is different in one sequence when compared to a related sequence when the two nucleic acids are aligned for maximal correspondence.

“Polynucleotide,” “polynucleotide sequence,” “nucleic acid sequence,” “nucleic acid fragment,” and “oligonucleotide” are used interchangeably herein. A polynucleotide is a polymer of nucleotides that is single- or multi-stranded, that optionally contains synthetic, non-natural, or altered RNA or DNA nucleotide bases. A DNA polynucleotide may be comprised of one or more strands of cDNA, genomic DNA, synthetic DNA, or mixtures thereof.

“Primer” refers to a synthetic or naturally occurring polynucleotide which acts as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers may be provided in double-stranded form, though the single-stranded form is more typically used. A primer can further contain a detectable label, for example a 5′ end label.

“Probe” refers to a synthetic or a naturally occurring polynucleotide that is sufficiently complementary (not necessarily fully complementary) to a polynucleotide of interest and forms a duplexed structure by hybridization with at least one strand of the polynucleotide of interest. Typically, probes are oligonucleotides from 10 to 50 nucleotides in length, but longer or shorter sequences can be employed. A probe can further contain a detectable label.

“Quantitative trait locus” or “QTL” refer to the genetic elements controlling a quantitative trait.

“Recombination frequency” is the frequency of a crossing over event (recombination) between two genetic loci. Recombination frequency can be observed by following the segregation of markers and/or traits during meiosis.

“Tolerance,” “improved tolerance,” “resistance,” and “improved resistance” are used interchangeably herein and refer to any type of increase in resistance or tolerance to, or any type of decrease in susceptibility to at least one herbicide. A “tolerant plant” or “tolerant plant variety” need not possess absolute or complete tolerance to at least one herbicide. Instead, a “tolerant plant,” “tolerant plant variety,” or a plant or plant variety with “improved tolerance” will have a level of resistance or tolerance to at least one herbicide which is higher than that of a comparable susceptible or less tolerant plant or variety.

“Self crossing” or “self pollination” or “selfing” is a process through which a breeder crosses a plant with itself; for example, a second generation hybrid F2 with itself to yield progeny designated F2:3.

“SNP” or “single nucleotide polymorphism” means a sequence variation that occurs when a single nucleotide (A, T, C, or G) in the genome sequence is altered or variable. “SNP markers” exist when SNPs are mapped to sites on the soybean genome.

The term “yield” refers to the productivity per unit area of a particular plant product of commercial value. For example, yield of soybean is commonly measured in bushels of seed per acre or metric tons of seed per hectare per season. Yield is affected by both genetic and environmental factors. Yield is the final culmination of all agronomic traits.

ALS-inhibiting herbicides are chemical compounds that inhibit acetolactate synthase and thus kill plants by inhibiting the production of the branched-chain aliphatic amino acids such as valine, leucine and isoleucine, which are required for DNA synthesis and cell growth. Herbicides inhibiting ALS can be categorized into five chemical subclasses based on general molecular structure: sulfonylureas, sulfonylaminocarbonyltriazolinones, triazolopyrimidines, pyrimidinyl(thio)benzoates and imidazolinones. Each of these chemical subclasses is well known in the art.

Sulfonylurea herbicide molecules comprise a sulfonylurea moiety (—S(O)2NHC(O)NH(R)). In sulfonylurea herbicides, the sulfonyl end of the sulfonylurea moiety is connected either directly or by way of an oxygen atom or an optionally substituted amino or methylene group to a typically substituted cyclic or acyclic group. At the opposite end of the sulfonylurea bridge, the amino group, which may have a substituent such as methyl (R being CH3) instead of hydrogen, is connected to a heterocyclic group, typically a symmetric pyrimidine or triazine ring, having one or two substituents such as methyl, ethyl, trifluoromethyl, methoxy, ethoxy, methylamino, dimethylamino, ethylamino and the halogens. In pyrimidinylsulfonylurea compounds the heterocyclic group is a symmetric pyrimidine ring. In triazinylsulfonylurea compounds the heterocyclic group is a symmetric triazine ring. Sulfonylurea herbicides can be in the form of the free acid or a salt. In the free acid form the sulfonamide nitrogen on the bridge is not deprotonated (i.e., S(O)2NHC(O)NH(R)), while in the salt form the sulfonamide nitrogen atom on the bridge is deprotonated (i.e., S(O)2N□C(O)NH(R)), and a cation is present, typically of an alkali metal or alkaline earth metal, most commonly sodium or potassium.

A representative sulfonylurea is shown in Formula I, including but not limited to any salts thereof,

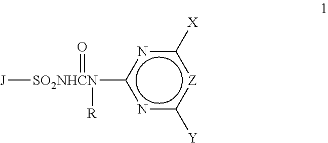

wherein J is selected from the group consisting of:

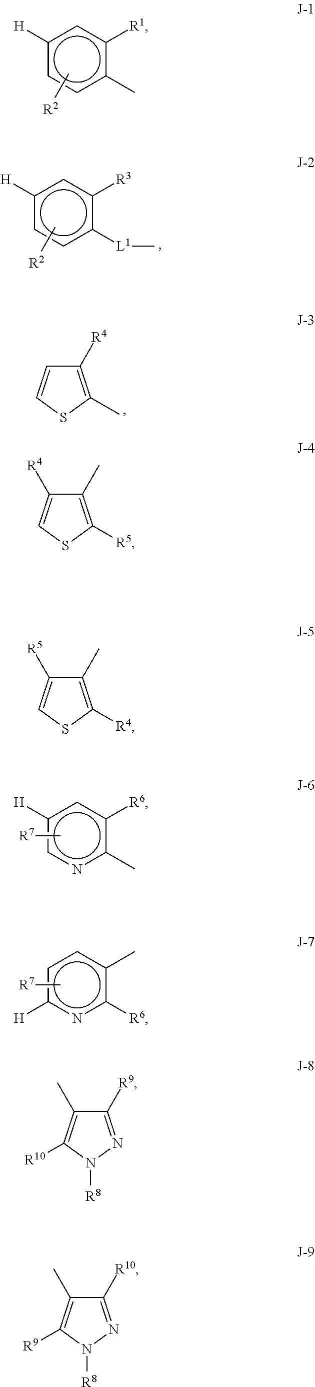

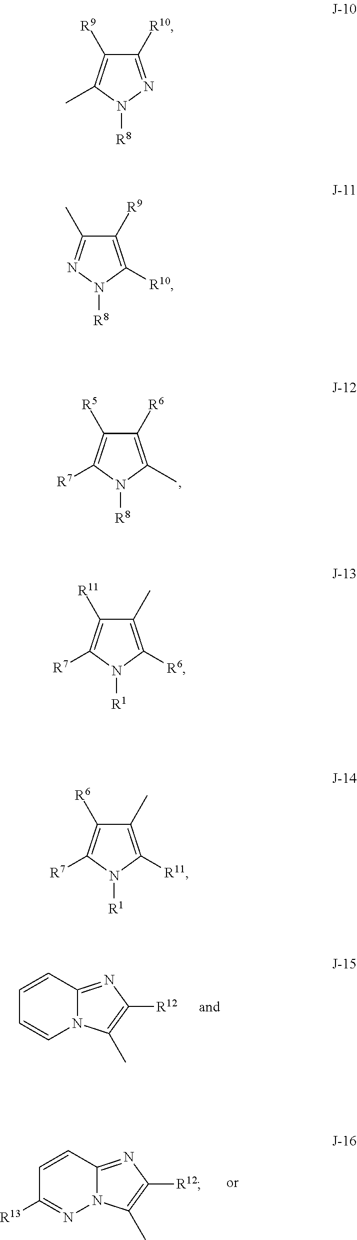

J is R¹⁴S0₃N(CH₃)—; R is H or CH₃;

R¹ is F, Cl Br, —NO₂, C₁-C₄ alkyl, C₁-C₄ haloalkyl, C₃-C₄ cycloalkyl, C₂-C₄ haloalkenyl, C₁-C₄ alkoxy, C₁-C₄ haloalkoxy, C₂-C₄ alkoxyalkoxy, CO₂R¹⁵, C(O)NR¹⁶R¹⁷, SO2NR¹⁸R¹⁹, S(O)ₙR²⁰, C(O)R²¹, CH₂CN or L;

R² is H, F, Cl, Br, I, cyano, CH₃, CF₃, CH₂NHS(O)₂CH₃, OCH₃, OCF₂H, SCH₃ or NHCHO;

R³ is Cl, nitro, CO₂CH₃, CO₂CH₂CH₃, C(O)CH₃, C(O)CH₂CH₃, C(O)-cyclopropyl, C(O)N(CH₃)₂, SO₂N(CH₃)₂, SO₂CH₃, SO₂CH₂CH₃, OCH₃ or OCH₂CH₃;

R⁴ is C₁-C₃ alkyl, C₁-C₂ haloalkyl, C₁-C₂ alkoxy, C₂-C₄ haloalkenyl, F, Cl, Br, nitro, CO₂R¹⁵, C(O)NR¹⁶R¹⁷, SO₂NR¹⁸R¹⁹, S(O)ₙR²⁰, C(O)R²¹ or L;

R⁵ is H, F, Cl, Br or CH₃;

R⁶ is C₁-C₃ alkyl optionally substituted with up to 3 F, up to 1 Cl and up to 1 C3-C₄ alkoxyacetyloxy, or R⁶ is C₁-C₂ alkoxy, C₁-C₂ haloalkoxy, C₂-C₄ haloalkenyl, F, Cl, Br, CO₂R¹⁵, C(O)NR¹⁶R¹⁷, SO₂NR¹⁸R¹⁹, S(O)ₙR²⁰, C(O)R²¹ or L;

R⁷ is H, F, Cl, CH₃ or CF₃;

R⁸ is H, C₁-C₃ alkyl or pyridinyl;

R⁹ is C₁-C₃ alkyl, C₁-C₂ alkoxy, F, Cl, Br, NO₂, CO₂R¹⁵, SO₂NR¹⁸R¹⁹, S(O)ₙR²⁰, OCF₂H, C(O)R²¹, C₂-C₄ haloalkenyl or L;

R¹⁰ is H, Cl, F, Br, C₁-C₃ alkyl or C₁-C₂ alkoxy;

R¹¹ is H, C₁-C₃ alkyl, C₁-C₂ alkoxy, C₂-C₄ haloalkenyl, F, Cl, Br, CO₂R¹⁵, C(O)NR¹⁶R¹⁷, SO₂NR¹⁸R¹⁹, S(O)ₙR²⁰, C(O)R²¹ or L;

R¹² is halogen, C₁-C₄ alkyl or C₁-C₃ alkylsulfonyl;

R¹³ is H or C₁-C₄ alkyl;

R¹⁴ is C₁-C₄ alkyl;

R¹⁵ is allyl, propargyl or oxetan-3-yl; or C₁-C₃ alkyl optionally substituted by up to 3 halogen, up to 1 C₁-C₂ alkoxy, and up to 1 cyano;

R¹⁶ is H, C₁-C₃ alkyl or C₁-C₂ alkoxy;

R¹⁷ is C₁-C₂ alkyl;

R¹⁸ is H, C₁-C₃ alkyl, C₁-C₂ alkoxy, allyl or cyclopropyl;

R¹⁹ is H or C₁-C₃ alkyl;

R²⁰ is C₁-C₃ alkyl, C₁-C₃ haloalkyl, allyl or propargyl;

R²¹ is C₁-C₄ alkyl or C₁-C₄ haloalkyl; or C₃-C₅ cycloalkyl optionally substituted by halogen;

n is 0, 1 or 2;

L is

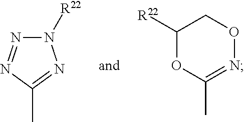

L¹ is CH₂, NH or O;

R²² is selected from the group H and C₁-C₃ alkyl;

X is selected from the group consisting of H, C₁-C₄ alkyl, C₁-C₄ alkoxy, C₁-C₄ haloalkoxy, C₁-C₄ haloalkyl, C₁-C₄ haloalkylthio, C₁-C₄ alkylthio, halogen, C₂-C₅ alkoxyalkyl, C₂-C₅ alkoxyalkoxy, amino, C₁-C₃ alkylamino and di(C₁-C₃ alkyl)amino;

Y is selected from the group consisting of H, C₁-C₄ alkyl, C₁-C₄ alkoxy, C₁-C₄ haloalkoxy, C₁-C₄ alkylthio, C₁-C₄ haloalkylthio, C₂-C₅ alkoxyalkyl, C₂-C₅ alkoxy-alkoxy, amino, C₁-C₃ alkylamino, di(C₁-C₃ alkyl)-amino, C₃-C₄ alkenyloxy, C₃-C₄ alkynyloxy, C₂-C₅ alkylthioalkyl, C₂-C₅ alkylsulfinylalkyl, C₂-C₅ alkylsulfonylalkyl, C₁-C₄ haloalkyl, C₂-C₄ alkynyl, C₃-C₅ cycloalkyl, azido and cyano; and

Z is selected from the group consisting of CH and N; provided that (i) when one or both of X and Y is O₁ haloalkoxy, then Z is CH; and (ii) when X is halogen, then Z is CH and Y is OCH₃, OCH₂CH₃, NHCH₃, N(CH₃)₂ or OCF₂H.

Sulfonylurea herbicides of Formula I wherein Z is CH are pyrimidinylsulfonylurea compounds. Sulfonylurea herbicides of Formula I wherein Z is N are triazinylsulfonylurea compounds.

Examples of sulfonylurea herbicides include amidosulfuron, azimsulfuron, bensulfuron-methyl, buthiuron, chlorimuron-ethyl, chlorsulfuron, cinosulfuron, cyclosulfamuron, ethametsulfuron-methyl, ethidimuron, ethoxysulfuron, flazasulfuron, flucetosulfuron, flupyrsulfuron-methyl (including sodium salt), foramsulfuron, halosulfuron-methyl, imazosulfuron, iodosulfuron-methyl (including sodium salt), mesosulfuron-methyl, metazosulfuron, metsulfuron-methyl, nicosulfuron, orthosulfamuron, oxasulfuron, primisulfuron-methyl, propyrisulfuron, prosulfuron, pyrazosulfuron-ethyl, rimsulfuron, sulfometuron-methyl, sulfosulfuron, tebuthiuron, thiazafluoron, thidiazuron, thifensulfuron-methyl, triasulfuron, tribenuron-methyl, trifloxysulfuron (including sodium salt), triflusulfuron-methyl, tritosulfuron, and any salts and derivatives thereof.

Sulfonylaminocarbonyltriazolinone herbicide molecules comprise a triazolinone moiety bonded through a C(O)NHS(O)₂ bridge to a typically substituted cyclic group. The bridge can be deprotonated (e.g., by bases) to form salts. Examples of sulfonylaminocarbonyltriazolinone herbicides include flucarbazone, propoxycarbazone, thiencarbazone-methyl, and any salts thereof.

Triazolopyrimidine herbicide molecules comprise a triazolopyrimidine moiety bonded to a S(O)₂NH bridge bonded at the other end to a typically substituted cyclic group. Deprotonation of the bridge (e.g., with bases) can form salts. Examples of triazolopyrimidine herbicides include cloransulam-methyl, diclosulam, florasulam, flumetsulam, metosulam, penoxsulam, pyroxsulam, and any salts thereof.

Pyrimidinyl(thio)benzoate herbicide molecules comprise pyrimidine ring bonded through an oxygen or sulfur atom to a benzene ring having a carboxylic acid or ester substituent. The carboxylic acid substituent can be deprotonated (e.g., by bases) to form salts. Examples of pyrimidinyl(thio)benzoate herbicides also identified as pyrimidinyl-(thio)ether herbicides include bispyribac, pyribenzoxim, pyriftalid, pyrithiobac, pyriminobac-methyl, and any salts thereof.

Imidazolinone herbicide molecules comprise an imidazolinone moiety. Examples of imidazolinone herbicides include imazapic, imazamethabenz-methyl, imazamox, imazapyr, imazaquin, imazethapyr, and any salts thereof including ammonium salts.

By providing plants having increased resistance to herbicides, particularly ALS-inhibiting herbicides, a wide variety of formulations can be employed for protecting plants from weeds to enhance crop growth and productivity. An herbicide can be used by itself for pre-emergence, post-emergence, pre-planting and/or at planting to control weeds in or adjacent to field. The herbicide can also be used as a seed treatment. A composition comprising an effective concentration of the herbicide can be applied directly to the seeds prior to or during the sowing of the seeds. Additives found in an herbicide formulation or composition include other herbicides, detergents, adjuvants, spreading agents, sticking agents, stabilizing agents, or the like. The herbicide formulation can be a wet or dry preparation and can include, but is not limited to, flowable powders, emulsifiable concentrates and liquid concentrates. The herbicide and herbicide formulations can be applied in accordance with conventional methods, for example, by spraying, irrigation, dusting, coating, and the like.

Many crop plants having resistance to one or more ALS-inhibiting herbicides based on natural resistance and selection, mutagenesis, and/or transgenic methods have been reported, including but not limited to soybean, rice, wheat, Brassica, sunflower, maize, sugarbeet, cotton, lettuce, tomato, tobacco (Tan et al. (2005) Pest Manag Sci 61:246-257) and barley (Lee et al. (2011) Proc Natl Acad Sci USA doi/10/1073/pnas.1105612108). Crop producers often rotate crops, and various ALS-inhibiting herbicides may have been used with the previous crop and there may be residual carryover in the field. Planting a crop with resistance to ALS-inhibiting herbicides can prevent crop injury from residual herbicide.

Sulfonylurea (SU) herbicides were discovered by DuPont chemist George Levitt in 1975 with the first SU herbicide products commercialized in 1982. SU's kill plants via inhibition of acetolactate synthase (ALS), a key enzyme in amino acid synthesis required for plant growth. This enzyme is not present in animals, and SU's have very low toxicity to non-target species making them valuable tools for controlling weeds in a wide variety of applications. It is well-known that different SU compounds have different activity and/or selectivity, for example some SU's are non-selective (kill all plants) and can be used for complete vegetation control. Other SU's are selective—i.e. they kill some plant species but are tolerated by others based on their differential ability to metabolize the SU before significant damage is done to ALS activity.

Two specific SU's (chlorimuron ethyl and thifensulfuron) are registered and used in a variety of herbicide formulations for selective weed control in soybean. At least some varieties of wild type soybeans have shown at least some level of tolerance to these SU's through metabolic inactivation. However resistance to SU's can also be conferred through specific mutations within ALS gene(s) that make the corresponding ALS enzyme(s) less susceptible to SU-inhibition but retain the vital catalytic ALS activity (see, e.g., weed management and herbicide information at www.agron.iastate.edu).

Several crop plants with tolerance or improved tolerance to ALS-inhibiting herbicides have been developed, including soybean, maize, wheat, rice, Brassica, and sunflower. Several of these were of interest based on resistance to the imidazolinone herbicides (for review, see Tan et al. (2005) Pest Manag Sci 61:246-257). Several different mutations have been observed to confer resistance to ALS-inhibiting herbicides, the most common of which include A122, P197, A205, W574, and S653 in the large subunit of ALS (using amino acid position numbering based on the A. thaliana sequence). These mutations confer resistance to one or more ALS-inhibiting herbicide classes, as summarized in Tan et al. (2005) Pest Manag Sci 61:246-257, herein incorporated by reference.

The genomic structure, ploidy, and number of gene loci vary extensively across plant species. The number of ALS large subunit genes varies across plant species, with Arabidopsis having one gene, corn having two genes, Brassica having 5 genes (three in the A genome, two in the C genome), rice having one gene, wheat having three genes, (one each on the A, B, and D genomes), and sunflower having 2-3 genes ((for review, see Tan et al. (2005) Pest Manag Sci 61:246-257).

In the mid 1980's, mutation breeding techniques were used to develop a soybean line (designated “W20”) having resistance to SU herbicides (Sebastian et al. (1989) Crop Sci 29:1403-1408; U.S. Pat. No. 5,084,082) determined to be based on modification of an ALS enzyme (no sequence data was available at that time). W20 is the original source of the soybean trait commercially identified as STS® (sulfonylurea tolerant soybean). STS® offers more selectivity and flexibility with SU's, for example with compounds specifically registered for soybean. STS® may also further provide options for the use of more efficacious and/or broader-spectrum SU's.

W20 was released to various soybean breeding companies in both North and South America in the late 1980's. Breeding and commercial use of the STS® trait expanded in the early 1990's, but slowed when transgenic glyphosate resistance became available. Although “stacked” (RR®+STS®) varieties have been available since the mid 1990's, there is renewed interest in SU resistance, alone or in combination with other herbicide tolerance stacks, to provide more options for the control of glyphosate resistant weeds. In particular, there is interest in elite soybean varieties having resistance to ALS-inhibiting herbicides. In some examples elite soybean varieties having resistance to ALS-inhibiting herbicides comprise other traits of interest including transgenic traits, native traits, and any combinations thereof.

Inheritance studies (Sebastian et al. (1989) Crop Sci 29:1403-1408, herein incorporated by reference in its entirety) indicated that the whole-plant SU resistance phenotype observed in W20 was conferred by a single semi-dominant mutation that co-segregated with in-vitro resistance of ALS enzyme activity to SU inhibition. Based on this evidence, the name “Als1” was given to the new allele conferring SU resistance in W20 soybean. Since Als1 provides a high level of SU resistance compared to wild type, simple and reliable phenotypic screens can distinguish between plants that are wild type (als1/als1), heterozygous (Als1/als1), and homozygous (Als1/Als1) in segregating breeding populations. Therefore, there was initially less need to develop genetic markers for marker assisted selection (MAS) of Als1, for example as contained in or derived from soybean line W20.

Subsequent to the development of W20, lines homozygous for Als1 were subjected to a second round of mutagenesis in an attempt to derive mutations that confer higher levels of SU resistance, or to broaden the number of effective compounds useful for weed control, for example as compared to lines comprising Als1 alone. From the second round of mutagenesis, a line “W4-4” was selected. W4-4 showed more resistance to SU's than the original W20 line, as demonstrated via both in vitro ALS enzyme activity assays and whole-plant assays (see, for example, U.S. Pat. No. 5,084,082, herein incorporated by reference in its entirety).

Segregation for SU resistance within populations derived from W4-4× wild type crosses indicated that W4-4 was homozygous for Als1 plus a second independently-segregating mutation. This second mutation was putatively identified as a second ALS gene and designated herein as “Als2”. This Als2 designation was originally based on ALS enzyme activity in vitro assays, and later confirmed by segregation studies as a specific mutation at a locus that was not genetically linked to the locus comprising the Als1 mutation. Further studies demonstrated that the combination of Als1 and Als2 provides higher crop safety for virtually every SU tested—including broad-spectrum SU's not yet registered for use in soybean.

Plants comprising more than one mutation in an ALS gene may or may not have increased resistance to ALS-inhibiting herbicides. In some cases, combinations of mutations have provided additive levels of resistance to an herbicide. In Brassica the PM1 and PM2 mutations were developed using microspore mutagenesis of B. napus (Swanson et al. (1989) Plant Cell Reports 7:83-87). The PM2 mutation is a single nucleotide change (G to T) of the 3′ end of the AHAS3 gene on the A genome (Rutledge et al. (1991) Mol Gen Genet. 229: 31-40), producing a Trp to Leu substitution at amino acid position 556 (Hattori et al. (1995) Mol Gen Genet. 246:419-425). The PM1 mutation on the C genome of B. napus (Rutledge et al. (1991) Mol Gen Genet. 229:31-40) is a single base change (G to A) in the AHAS1 gene resulting in a Ser to Asn amino acid change at position 638 in the protein (Sathasivan et al. (1991) Plant Physiol 97:1044-1050; Hattori et al. (1992) Mol Gen Genet. 232:167-173; US2004/0142353; and US2004/0171027). These two mutations, PM1 (AHAS1) and PM2 (AHAS3), were reported to provide additive levels of tolerance to imidazolinone herbicides (Swanson et al. (1989) Theor Appl Genet. 78:525-530). In tobacco (Nicotiana tabacum L.) two different ALS mutations (csrl-1 and ahas3r) were provided transgenically, and plants comprising either one of the two genes alone or both genes together were screened with sulfonylurea compounds (chlorsulfuron and DPX-R9674). Seedlings carrying both genes were intermediate in response to increasing herbicide concentration relative to seedlings having either gene alone. However, synergy has been observed in Brassica plants having two or more AHASL subunit single mutations as reported in EP2546348A1.

The increasing incidence of glyphosate resistant weeds establishes the need for addition weed control options, for example in soybean. Incorporation of Als1 and/or Als2 mutations, for example as found in W4-4 soybean, provides additional options for herbicide tolerance and weed control for soybean crops. Breeding efforts to introgress and/or stack Als1 and Als2, optionally with other desirable traits (native and/or transgenic) would be greatly facilitated by the development of co-dominant markers (e.g., SNPs) for MAS. These markers eliminate the need for phenotypic screening protocols capable of differentiating the numerous zygosity states at the ALS1 and/or ALS2 loci possible in breeding populations. Further, markers can identify the exact zygotic condition of single plants with high certainty, and would reduce the need for progeny testing to confirm that breeding lines are “fixed” (homozygous and homogeneous) for the Als1 and/or the Als2 alleles. Efforts to quickly backcross multiple genes into target elite germplasm would also be simplified with MAS. Hence, studies were conducted to determine the DNA sequence of the Als1 and Als2 alleles. These sequences were used to facilitate development of broadly-applicable SNP markers for MAS for these alleles. Since W4-4 was known to contain both Als1 and Als2 mutations based on its breeding history and SU-resistance phenotype, sequencing of each mutation could be accomplished by comparing the sequence of ALS genes in W4-4 to the sequence of ALS genes in wild-type soybean lines.

In some examples, any herbicide can be used in the methods and/or compositions comprising ALS inhibitor-tolerant soybean provided, or the area of cultivation containing said crop plant. Classifications of herbicides (i.e., the grouping of herbicides into classes and subclasses) are well-known and include classifications by HRAC (Herbicide Resistance Action Committee) and WSSA (the Weed Science Society of America) (see also, Retzinger & Mallory-Smith (1997) Weed Technol 11:384-393). Herbicides can be classified by their mode of action and/or site of action, by the time at which they are applied (e.g., pre-emergent or post-emergent), by the method of application (e.g., foliar application or soil application), or by how they are taken up by or affect the plant, or combinations of these criteria. For example, thifensulfuron-methyl and tribenuron-methyl are applied to the foliage and are generally metabolized there, while rimsulfuron and chlorimuron-ethyl are generally taken up through both the roots and foliage of a plant. Herbicides can be classified in various ways, including by mode of action and/or site of action.

A herbicidally effective amount of the compound(s) is determined by a number of well established factors, including but not limited to formulation selected, method of application, amount and type of vegetation present, weather conditions, soil conditions, soil characteristics, growing conditions, other chemicals present in or on the field, seed, and/or crop, and the like. An effective amount or an effective concentration is an amount sufficient to kill or inhibit the growth of a similar, wild-type, plant, plant tissue, plant cell, microspore, or host cell, but that does not kill or severely inhibit the growth of herbicide-resistant plants, tissues, cells, germplasm, or seed. Typically, an effective amount corresponds to a concentration or application rate routinely used in agricultural production systems to kill weeds of interest. Such an amount is known to those of ordinary skill in the art, or can be easily determined using methods known in the art. The effective amount of an herbicide in an agricultural production system might be substantially different than an effective amount of an herbicide for a plant culture system. In general, a herbicidally effective amount of a compound is applied at rates from about 0.001 to 20 kg/ha with a preferred rate range of 0.004 to 0.25 kg/ha. One skilled in the art can easily determine application rates necessary for the desired level of weed control for any given crop and set of conditions.

The control of undesired vegetation is understood as meaning the killing of weeds and/or otherwise retarding or inhibiting the normal growth of the weeds. Weeds, in the broadest sense, are any and all those plants which grow in locations where they are undesired. In some examples weeds can include crop plants that are growing in an undesired location. For example, a volunteer maize plant that is in a field that predominantly comprises soybean plants can be considered a weed. Typical weeds include, for example, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum, Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera.

Synergy is the interaction of elements that when combined produce a total effect that is greater than the sum of the individual elements. In order to identify any synergy between two or more individual elements, one must be able to measure the impact of each individual element alone as well as the effect of any combination of two or more of the elements. Many methods are known in the art to detect and analyze whether combinations of factors have an additive, an antagonistic, or a synergistic effect. Berenbaum ((1989) Pharmacol Rev 41:93-141, herein incorporated by reference) provides a thorough review and comparison of various methods. One such method for analysis of interactions between two or more factors is the isobole method. The isobole method is independent of the mechanism of action and is based on an empirical model. The isobole method uses the dose response of biologically active compounds in a mixture, and uses equally effective (isoeffective) doses for each of the compounds to build an isobole graph. Typically, dose response assays are used to estimate the biological activity. The observed response is a measure of some characteristic of the subject which indicates the biological activity of the stimulus. The relationship between dose and response is used to calculate the expected concentration of a sample predicted to elicit an X % response (ECx). For example, the EC50 is the concentration of a compound predicted to elicit a 50% response. =Well-designed dose response experiments should strive to provide a wide-range of responses, for example a low response, e.g., about 20% of control, to very high response, e.g., about 80% control. The EC level used for analysis should be determined by the data, for example if the maximum response observed is 30% of the control, the isobole graph should be determined using an EC value within the range of responses, for example EC20, rather than an EC50 estimate. Using the isobole method, interaction is detected when the effect of a combination of agents differs from that expected from their individual dose response curves.

Acetolactate synthase (ALS; EC 4.1.3.18) is the first enzyme that catalyzes the biochemical synthesis of the branched chain amino acids valine, leucine and isoleucine (Singh (1999) “Biosynthesis of valine, leucine and isoleucine: in: Singh (Ed) Plant amino acids (Marcel Dekker Inc. NY, N.Y. pp 227-247). ALS is the site of action of five structurally diverse herbicide families including the sulfonylureas (LaRossa & Falco (1984) Trends Biotechnol 2:158-161), the imidazolinones (Shaner et al. (1984) Plant Physiol 76:545-546), the triazolopyrimidines (Subramanian & Gerwick (1989) “Inhibition of acetolactate synthase by triazolopyrimidines” in Whitaker & Sonnet (Ed) Biocatalysis in Agricultural Biotechnology (ACS Symposium Series, American Chemical Society Washington, D.C. pp 277-288)), and the pyrimidyloxybenzoates (Subramanian et al. (1990) Plant Physiol 94: 239-244). Imidazolinone and sulfonylurea herbicides are widely used in modern agriculture due to their effectiveness at very low application rates and relative non-toxicity in animals. By inhibiting ALS activity, these families of herbicides prevent further growth and development of susceptible plants including many weed species.

Herbicidal formulations of interest may also include herbicides comprising imidazolinones. Due to their high effectiveness and low-toxicity, these compounds are favored for application by spraying over the top of a wide area of vegetation. The ability to spray an herbicide over the top of a wide range of vegetation decreases the costs associated with plantation establishment and maintenance and decreases the need for site preparation prior to use of such chemicals. Spraying over the top of a desired tolerant species also results in the ability to achieve maximum yield potential of the desired species due to the absence of competitive species. However, the ability to use such spray-over techniques is dependent upon the presence of imidazolinone resistant species of the desired vegetation in the spray-over area. Among the major agricultural crops, some leguminous species such as soybean are naturally resistant to imidazolinone herbicides due to their ability to rapidly metabolize the herbicide compounds (Shaner & Robinson (1985) Weed Sci 33:469-471). Differential sensitivity to the imidazolinone herbicides is dependent on the chemical nature of the particular herbicide and differential metabolism of the compound from a toxic to a non-toxic form in each plant (Shaner et al. (1984) Plant Physiol 76:545-546; Brown et al. (1987) Pestic Biochem Physiol 27:24-29). Other plant physiological differences such as absorption and translocation also play an important role in sensitivity (Shaner & Robinson (1985) Weed Sci 33:469-471). Other reports of plant resistance to imidazolinone herbicides include U.S. Pat. No. 5,013,659, U.S. Pat. No. 5,731,180, and U.S. Pat. No. 5,767,361.

The AHAS enzyme is comprised of two subunits: a large subunit (catalytic role) and a small subunit (regulatory role) (Duggleby & Pang (2000) J Biochem Mol Biol 33:1-36). The AHAS large subunit (AHASL) may be encoded by a single gene, as found in Arabidopsis and rice, or by multiple gene family members such as in maize, canola, and cotton. Specific, single-nucleotide substitutions in the large subunit have been found to confer at least some insensitivity to one or more classes of herbicides (Chang & Duggleby (1998) Biochem J 333:765-777).

Bread wheat, Triticum aestivum L., contains three homologous acetohydroxyacid synthase large subunit genes. Each of these exhibit significant expression based on herbicide response and biochemical data from mutants in each of the three genes (Ascenzi et al. (2003) International Society of Plant Molecular Biologists Congress, Barcelona, Spain, Ref. No. S10-17). The coding regions of these three genes have high sequence homology at the nucleotide level (WO 03/014357). Sequencing AHASL genes from several varieties of Triticum aestivum indicated herbicide tolerance in most IMI-tolerant (imidazolinone-tolerant) lines was caused by a serine to asparagine substitution at a position equivalent to the serine at amino acid 653 in Arabidopsis thaliana (WO 03/014357). This mutation is a single nucleotide polymorphism (SNP) in the DNA sequence encoding the AHASL protein.

Kolkman et al. ((2004) Theor Appl Genet. 109:1147-1159) reported identification, cloning, and sequencing for three AHASL genes (AHASL1, AHASL2, and AHASL3) from herbicide-resistant and wild type genotypes of sunflower (Helianthus annuus L.). Kolkman et al. reported that herbicide-resistance was due either to a Pro197Leu substitution (using Arabidopsis AHASL amino acid numbering), or an Ala205Val substitution in the AHASL1 protein and that each of these substitutions provided resistance to both imidazolinone and sulfonylurea herbicides.

Many effective herbicide formulations and herbicide combinations comprising an ALS-inhibiting herbicide are commercially available, including but not limited to MATRIX® (rimsulfuron), BASIS® (rimsulfuron and thifensulfuron-methyl), OUST® (sulfometuron-methyl), RESOLVE® (rimsulfuron), EXPRESS® (tribenuron), PURSUIT® (imazethpyr), HARMONY® (thifensulfuron), SYNCHRONY® (chlorimuron-ethyl and thifensulfuron-methyl), BRUSH-OFF® (metsulfuron-methyl), GLEAN® (chlorsulfuron), LOGRAN® (triasulfuron), FIRST RATE® (chloransulam-methyl), SCEPTOR® (imazaquin), BEYOND ® (imazamox), ARSENAL® (imazapyr), CLASSIC® (chlorimuron-ethyl), ALLY® (metsulfuron-methyl), DILIGENT™ (rimsulfuron, chlorimuron-ethyl, and flumioxazin), and LIGHTNING® (imazapyr and imazethapyr).

Many effective herbicide formulations and herbicide combinations comprising non-ALS-inhibiting herbicides are commercially available, including but not limited to TOUCHDOWN® (glyphosate), WEATHERMAX® (glyphosate), ROUNDUP® (glyphosate isopropylamine), LORSBAN® (chlorpyrifos), ENLIST® (2,4-D and glyphosate), BASTA® (glufosinate-ammonium), FINALE® (glufosinate-ammonium), RELY® (glufosinate-ammonium), BALANCE® (isoxaflutole and cyprosulfamide), DILIGENT™ (rimsulfuron, chlorimuron-ethyl, and flumioxazin), and CORVUS® (isoxaflutole).

In some cases, an herbicide-tolerance gene will also confer tolerance to other herbicides or chemicals in the same class or subclass. Thus, in some examples a soybean plant provided has tolerance to more than one herbicide or chemical in the same class or subclass, such as, for example, a sulfonylurea, an imidazolinone, an inhibitor of PPO, an HPPD, a metribuzin, or a synthetic auxin.

Typically, soybean plants provided can tolerate treatment with different types of herbicides (i.e., herbicides having different modes of action and/or different sites of action) as well as with higher amounts of herbicides than previously known plants, thereby permitting improved weed management strategies that are recommended in order to reduce the incidence and prevalence of herbicide-tolerant weeds. Specific herbicide combinations can be employed to effectively control weeds.

A soybean plant that can be selected for use in crop production based on the prevalence of herbicide-tolerant weed species in the area where the crop is to be grown is provided. Methods are known in the art for assessing the herbicide tolerance of various weed species. Weed management techniques are also known in the art, such as for example, utilizing crop rotation using a crop that is tolerant to a herbicide to which the local weed species are not tolerant. A number of entities monitor and publicly report the incidence and characteristics of herbicide-tolerant weeds, including the Herbicide Resistance Action Committee (HRAC), the Weed Science Society of America, and various state agencies (see, e.g., herbicide tolerance scores for various broadleaf weeds from the 2004 Illinois Agricultural Pest Management Handbook), and one of skill is able to use this information to determine which crop and herbicide combinations should be used in a particular location. These groups also publish advice and guidelines for preventing the development and/or appearance of and controlling the spread of herbicide tolerant weeds (see, e.g., Owen & Hartzler (2004) Herbicide Manual for Agricultural Professionals, Pub. WC 92 Revised (Iowa State University Extension, Iowa State University, Ames, Iowa); Weed Control for Corn, Soybeans, and Sorghum, Ch 2 of “2004 Illinois Agricultural Pest Management Handbook” (University of Illinois Extension, University of Illinois at Urbana-Champaign, Illinois)); Weed Control Guide for Field Crops, MSU Extension Bulletin E434 (Michigan State University, East Lansing, Mich., USA)).

A soybean plant, germplasm, plant part, or seed further comprising resistance to another herbicidal formulation is provided. For example, the herbicidal formulation can comprise a compound selected from the group consisting of a glyphosate, a hydroxyphenylpyruvatedioxygenase (HPPD) inhibitor, a sulfonamide, an imidazolinone, a bialaphos, a phosphinothricin, a metribuzin, a mesotrione, an isoxaflutole, an azafenidin, a butafenacil, a sulfosate, a glufosinate, a dicamba, a 2,4-D, and a protox inhibitor. In some examples, resistance to the herbicidal formulation is conferred by a transgene.

Glyphosate resistance can be conferred from genes including but not limited to EPSPS, GAT, GOX, and the like, such as described in U.S. Pat. Nos. 6,248,876; 5,627,061; 5,804,425; 5,633,435; 5,145,783; 4,971,908; 5,312,910; 5,188,642; 4,940,835; 5,866,775; 6,225,114; 6,130,366; 5,310,667; 4,535,060; 4,769,061; 5,633,448; 5,510,471; RE36,449; RE37,287 E; 5,491,288; 5,776,760; 5,463,175; 8,044,261; 7,527,955; 7,666,643; 7,998,703; 7,951,995; 7,968,770; 8,088,972, 7,863,503; and US20030083480; WO 97/04103; WO 00/66746; WO 01/66704; and WO 00/66747, which are each incorporated herein by reference in their entireties for all purposes. Additionally, glyphosate tolerant plants can be generated through the selection of naturally occurring mutations that impart tolerance to glyphosate.

HPPD resistance can be conferred by genes including exemplary sequences disclosed in U.S. Pat. Nos. 6,245,968; 6,268,549; and 6,069,115; and WO 99/23886, which are each incorporated herein by reference in their entireties for all purposes. Mutant hydroxyphenylpyruvatedioxygenases having this activity are also known. For further examples see US20110185444 and US20110185445.

Resistance to auxins, such as 2,4-D or dicamba, can be provided by polynucleotides as described, for example, in WO2005/107437, US20070220629, and U.S. Pat. No. 7,838,733 and in Herman et al. (2005) J. Biol. Chem. 280:24759-24767, each which is herein incorporated by reference.

Resistance to PPO-inhibiting herbicides can be provided as described in U.S. Pat. Nos. 6,288,306; 6,282,837; and 5,767,373; and WO 01/12825, each of which is herein incorporated by reference. Plants containing such polynucleotides can exhibit improved tolerance to any of a variety of herbicides which target the protox enzyme. Resistance can also be conferred as described in US20100186131; US20110185444; US20100024080, each of which is herein incorporated by reference.

The development of plants containing an exogenous phosphinothricin acetyltransferase which confers resistance to glufosinate, bialaphos, or phosphinothricin is described, for example, in U.S. Pat. Nos. 5,969,213; 5,489,520; 5,550,318; 5,874,265; 5,919,675; 5,561,236; 5,648,477; 5,646,024; 6,177,616; and 5,879,903, which are each incorporated herein by reference in their entireties for all purposes. Mutant phosphinothricin acetyltransferase having this activity are also known in the art.

In some examples, the plant or germplasm further comprises a trait selected from the group consisting of drought tolerance, stress tolerance, disease resistance, herbicide resistance, enhanced yield, modified oil, modified protein, tolerance to chlorotic conditions, and insect resistance, or any combination thereof. In some examples, the trait is selected from the group consisting of brown stem rot resistance, charcoal rot drought complex resistance, Fusarium resistance, Phytophthora resistance, stem canker resistance, sudden death syndrome resistance, Scierotinia resistance, Cercospora resistance, anthracnose resistance, target spot resistance, frogeye leaf spot resistance, soybean cyst nematode resistance, root knot nematode resistance, rust resistance, high oleic content, low linolenic content, aphid resistance, stink bug resistance, and iron chlorosis deficiency tolerance, or any combination thereof. In some examples, one or more of the traits is conferred by one or more transgenes, by one or more native loci, or any combination thereof. Examples of markers and loci conferring improved iron chlorosis deficiency tolerance are disclosed in US20110258743, U.S. Pat. No. 7,582,806, and U.S. Pat. No. 7,977,533, each of which is herein incorporated by reference. Various disease resistance loci and markers are disclosed, for example, in WO1999031964, U.S. Pat. No. 5,948,953, U.S. Pat. No. 5,689,035, US20090170112, US20090172829, US20090172830, US20110271409, US20110145953, U.S. Pat. No. 7,642,403, U.S. Pat. No. 7,919,675, US20110131677, U.S. Pat. No. 7,767,882, U.S. Pat. No. 7,910,799, US20080263720, U.S. Pat. No. 7,507,874, US20040034890, US20110055960, US20110185448, US20110191893, US20120017339, U.S. Pat. No. 7,250,552, U.S. Pat. No. 7,595,432, U.S. Pat. No. 7,790,949, U.S. Pat. No. 7,956,239, U.S. Pat. No. 7,968,763, each of which is herein incorporated by reference. Markers and loci conferring improved yield are provided, for example, in U.S. Pat. No. 7,973,212 and WO2000018963, each of which is herein incorporated by reference. Markers and loci conferring improved resistance to insects are disclosed in, for example, US20090049565, U.S. Pat. No. 7,781,648, US20100263085, U.S. Pat. No. 7,928,286, U.S. Pat. No. 7,994,389, and WO2011116131, each of which is herein incorporated by reference. Markers and loci for modified soybean oil content or composition are disclosed in, for example, US20120028255 and US20110277173, each of which is herein incorporated by reference. Methods and compositions to modified soybean oil content are described in, for example, WO2008147935, U.S. Pat. No. 8,119,860; U.S. Pat. No. 8,119,784; U.S. Pat. No. 8,101,189; U.S. Pat. No. 8,058,517; U.S. Pat. No. 8,049,062; U.S. Pat. No. 8,124,845, U.S. Pat. No. 7,790,959, U.S. Pat. No. 7,531,718, U.S. Pat. No. 7,504,563, and U.S. Pat. No. 6,949,698, each of which is herein incorporated by reference. Markers and loci conferring tolerance to nematodes are disclosed in, for example, US20090064354, US20090100537, US20110083234, US20060225150, US20110083224, U.S. Pat. No. 5,491,081, U.S. Pat. No. 6,162,967, U.S. Pat. No. 6,538,175, U.S. Pat. No. 7,872,171, U.S. Pat. No. 6,096,944, and U.S. Pat. No. 6,300,541, each of which is herein incorporated by reference. Resistance to nematodes may be conferred using a transgenic approach as described, for example, in U.S. Pat. No. 6,284,948 and U.S. Pat. No. 6,228,992, each of which is herein incorporated by reference. Plant phenotypes can be modified using isopentyl transferase polynucleotides as described, for example, in U.S. Pat. No. 7,553,951 and U.S. Pat. No. 7,893,236, each of which is herein incorporated by reference.

Soybean plants, germplasm, cells, or seed may be evaluated by any method to determine the presence of a mutated ALS polynucleotide or polypeptide. Methods include phenotypic evaluations, genotypic evaluations, or combinations thereof. The progeny plants may be evaluated in subsequent generations for herbicide resistance, and other desirable traits. Resistance to ALS-inhibitor herbicides may be evaluated by exposing plants, cells, or seed to one or more appropriate ALS-inhibitor herbicides and evaluating herbicide injury. Genotypic evaluation of the plants, germplasm, cells or seeds includes using techniques such as isozyme electrophoresis, restriction fragment length polymorphisms (RFLPs), randomly amplified polymorphic DNAs (RAPDs), arbitrarily primed polymerase chain reaction (AP-PCR), DNA amplification fingerprinting (DAF), sequence characterized amplified regions (SCARs), amplified fragment length polymorphisms (AFLPs), simple sequence repeats (SSRs), single nucleotide polymorphisms (SNPs), insertions or deletions (indels), sequencing, northern blots, southern blots, marker profiles, and the like.

Provided are markers, marker combinations, and/or marker profiles associated with tolerance of soybean plants to at least one herbicide, as well as related primers and/or probes and methods for the use of any of the foregoing for identifying and/or selecting soybean plants with improved tolerance to at least one herbicide. A method for determining the presence or absence of at least one allele of a particular marker or combination of markers associated with tolerance to at least one herbicide comprises analyzing genomic DNA from a soybean plant or germplasm to determine if at least one, or a plurality, of such markers is present or absent and if present, and determining the allelic form of the marker(s). In some examples a plurality of markers on a single linkage group are investigated, and the markers present in the particular plant or germplasm can be used to determine a haplotype for that plant/germplasm. In other examples a plurality of markers on distinct linkage groups are investigated, and the markers present in the particular plant or germplasm can be used to determine a marker profile for that plant/germplasm.

Soybean seeds, plants, and plant parts comprising a mutant acetolactate synthase gene may be cleaned and/or treated. The resulting seeds, plants, or plant parts produced by the cleaning and/or treating process(es) may exhibit enhanced yield characteristics.

Enhanced yield characteristics can include one or more of the following: increased germination efficiency under normal and/or stress conditions, improved plant physiology, growth and/or development, such as water use efficiency, water retention efficiency, improved nitrogen use, enhanced carbon assimilation, improved photosynthesis, and accelerated maturation, and improved disease and/or pathogen tolerance. Yield characteristics can furthermore include enhanced plant architecture (under stress and non-stress conditions), including but not limited to early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield characteristics include seed composition, such as carbohydrate content, protein content, oil content and composition, nutritional value, reduction in anti-nutritional compounds, improved processability and better storage stability.

Cleaning a seed or seed cleaning refers to the removal of impurities and debris material from the harvested seed. Material to be removed from the seed includes but is not limited to soil, and plant waste, pebbles, weed seeds, broken soybean seeds, fungi, bacteria, insect material, including insect eggs, larvae, and parts thereof, and any other pests that exist with the harvested crop. The terms cleaning a seed or seed cleaning also refer to the removal of any debris or low quality, infested, or infected seeds and seeds of different species that are foreign to the sample.

Treating a seed or applying a treatment to a seed refers to the application of a composition to a seed as a coating or otherwise. The composition may be applied to the seed in a seed treatment at any time from harvesting of the seed to sowing of the seed. The composition may be applied using methods including but not limited to mixing in a container, mechanical application, tumbling, spraying, misting, and immersion. Thus, the composition may be applied as a powder, a crystalline, a ready-to-use, a slurry, a mist, and/or a soak. For a general discussion of techniques used to apply fungicides to seeds, see “Seed Treatment,” 2d ed., (1986), edited by KA Jeffs (chapter 9), herein incorporated by reference in its entirety. The composition to be used as a seed treatment can comprise one or more of a pesticide, a fungicide, an insecticide, a nematicide, an antimicrobial, an inoculant, a growth promoter, a polymer, a flow agent, a coating, or any combination thereof. General classes or family of seed treatment agents include triazoles, anilides, pyrazoles, carboxamides, succinate dehydrogenase inhibitors (SDHI), triazolinthiones, strobilurins, amides, and anthranilic diamides. In some examples, the seed treatment comprises trifloxystrobin, azoxystrobin, metalaxyl, metalaxyl-m, mefenoxam, fludioxinil, imidacloprid, thiamethoxam, thiabendazole, ipconazole, penflufen, sedaxane, prothioconazole, picoxystrobin, penthiopyrad, pyraclastrobin, xemium, Rhizobia spp., Bradyrhizobium spp. (e.g., B. japonicum), Bacillus spp. (e.g., B. firmus, B. pumilus, B. subtilus), lipo-chitooligosaccharide, clothianidin, cyantraniliprole, chlorantraniliprole, abamectin, and any combination thereof. In some examples the seed treatment comprises trifloxystrobin, metalaxyl, imidacloprid, Bacillus spp., and any combination thereof. In some examples the seed treatment comprises picoxystrobin, penthiopyrad, cyantraniliprole, chlorantraniliprole, and any combination thereof. In some examples, the seed treatment improves seed germination under normal and/or stress environments, early stand count, vigor, yield, root formation, nodulation, and any combination thereof. In some examples seed treatment reduces seed dust levels, insect damage, pathogen establishment and/or damage, plant virus infection and/or damage, and any combination thereof.

Genetic elements or genes located on a single chromosome segment are physically linked. In some examples, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosome segment are also genetically linked, typically within a genetic recombination distance of less than or equal to 50 centimorgans (cM), e.g., about 49, 40, 30, 20, 10, 9, 8, 7, 6, 5, 4, 3, 2, 1, 0.75, 0.5, or 0.25 cM or less. That is, two genetic elements within a single chromosome segment undergo recombination during meiosis with each other at a frequency of less than or equal to about 50%, e.g., about 49%, 40%, 30%, 20%, 10%, 9%, 8%, 7%, 6%, 5%, 4%, 3%, 2%, 1%, 0.75%, 0.5%, or 0.25% or less. Closely linked markers display a cross over frequency with a given marker of about 10% or less (the given marker is within about 10 cM of a closely linked marker). Put another way, closely linked loci co-segregate at least about 90% of the time.

In certain examples, plants or germplasm are identified that have at least one favorable allele, marker, marker profile, and/or haplotype that positively correlates with tolerance or improved tolerance. However, in other examples, it is useful to identify alleles, markers, marker profiles, and/or haplotypes that negatively correlate with tolerance, for example to eliminate such plants or germplasm from subsequent rounds of breeding.

Any marker associated with a herbicide tolerance QTL is useful. Further, any suitable type of marker can be used, including Restriction Fragment Length Polymorphisms (RFLPs), Single Sequence Repeats (SSRs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Single Nucleotide Polymorphisms (SNPs). Additionally, other types of molecular markers known in the art or phenotypic traits may also be used in the methods.

Markers that map closer to an herbicide tolerance QTL are generally preferred over markers that map farther from such a QTL. Marker loci are especially useful when they are closely linked to an herbicide tolerance QTL. Thus, in one example, marker loci display an inter-locus cross-over frequency of about 10% or less, about 9% or less, about 8% or less, about 7% or less, about 6% or less, about 5% or less, about 4% or less, about 3% or less, about 2% or less, about 1% or less, about 0.75% or less, about 0.5% or less, or about 0.25% or less with a herbicide tolerance QTL to which they are linked. Thus, the loci are separated from the QTL to which they are linked by about 10 cM, 9 cM, 8 cM, 7 cM, 6 cM, 5 cM, 4 cM, 3 cM, 2cM, 1cM, 0.75 cM, 0.5 cM, or 0.25 cM or less. In certain examples, multiple marker loci that collectively make up a haplotype are investigated, for instance 2, 3, 4, 5, 6, 7, 8, 9, 10, 11, 12, 13, 14, 15, 16, 17, 18, 19, 20, 21, 22, 23, 24, 25, 26, 27, 28, 29, 30, or more marker loci.

Both chromosome number and linkage group identifiers have been used to describe soybean genome based on genetic mapping data, physical mapping data, and sequencing data and assemblies. Linkage group lengths in cM are based on the Soybean Consensus Map 3.0 produced by Perry Cregan's group at the USDA-ARS Soybean Genomics and Improvement Lab. The 11 initial linkage group to chromosome number assignments were made by Ted Hymowitz's group (Zou et al. (2003) Theor Appl Genet. 107:745-750 and citations therein). The remaining 9 were given chromosome numbers in decreasing order of linkage group genetic length. Based on this system, linkage group C1 is chromosome 4 (Gm04), and linkage group C2 is chromosome 6 (Gm06). The soybean chromosome number to linkage group assignments can be found at Soybase (see, e.g., soybase.org/LG2Xsome.php).

Large numbers of soybean genetic markers have been mapped and linkage groups created, for example as described in Cregan et al., “An Integrated Genetic Linkage Map of the Soybean Genome” (1999) Crop Sci 39:1464-90, and Choi et al., “A Soybean Transcript Map: Gene Distribution, Haplotype and Single-Nucleotide Polymorphism Analysis” (2007) Genetics 176:685-96. Many soybean markers are publicly available at the USDA affiliated soybase website (www.soybase.org). All markers are used to define a specific locus on the soybean genome. Each marker is therefore an indicator of a specific segment of DNA, having a unique nucleotide sequence. The map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage and/or under environmental conditions where the trait can be expressed. Molecular markers have been widely used to determine genetic composition in soybeans.

In addition to the markers discussed herein, information regarding useful soybean markers can be found, for example, on the USDA's Soybase website, available at www.soybase.org. One of skill in the art will recognize that the identification of favorable marker alleles may be germplasm-specific. One of skill will also recognize that methods for identifying the favorable alleles are routine and well known in the art, and furthermore, that the identification and use of such favorable alleles is well within the scope of the invention.

The use of marker assisted selection (MAS) to select a soybean plant or germplasm based upon detection of a particular marker or haplotype of interest is provided. For instance, in certain examples, a soybean plant or germplasm possessing a certain predetermined favorable marker allele, marker profile, or haplotype will be selected via MAS. Using MAS, soybean plants or germplasm can be selected for markers or marker alleles that positively correlate with tolerance, without actually raising soybean and measuring for tolerance (or, contrawise, soybean plants can be selected against if they possess markers that negatively correlate with tolerance). MAS is a powerful tool to select for desired phenotypes and for introgressing desired traits into cultivars of soybean (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

In some examples, molecular markers are detected using a suitable amplification-based detection method. Typical amplification methods include various polymerase based replication methods, including the polymerase chain reaction (PCR), ligase mediated methods, such as the ligase chain reaction (LCR), and RNA polymerase based amplification (e.g., by transcription) methods. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. For example, oligonucleotides can be synthesized chemically according to the solid phase phosphoramidite triester method described by Beaucage & Caruthers (1981) Tetrahedron Letts 22:1859-1862, e.g., using a commercially available automated synthesizer, e.g., as described in Needham-VanDeventer, et al. (1984) Nucl Acids Res 12:6159-6168. Oligonucleotides, including modified oligonucleotides, can also be ordered from a variety of commercial sources known to persons of skill in the art.

It will be appreciated that suitable primers and probes to be used can be designed using any suitable method. It is not intended that the invention be limited to any particular primer, primer pair, or probe. For example, primers can be designed using any suitable software program, such as LASERGENE® or Primer3.

It is not intended that the primers be limited to generating an amplicon of any particular size. For example, the primers used to amplify the marker loci and alleles herein are not limited to amplifying the entire region of the relevant locus. In some examples, marker amplification produces an amplicon at least 20 nucleotides in length, at least 50 nucleotides in length, at least 100 nucleotides in length, at least 200 nucleotides in length, at least 300 nucleotides in length, at least 400 nucleotides in length, at least 500 nucleotides in length, at least 1000 nucleotides in length, at least 2000 nucleotides in length, or greater than 2000 nucleotides in length.

PCR, RT-PCR, and LCR are common amplification and amplification-detection methods for amplifying nucleic acids of interest (e.g., those comprising marker loci), facilitating detection of the markers. Details regarding the use of these and other amplification methods are well known in the art and can be found in any of a variety of standard texts. Details for these techniques can also be found in numerous journal and patent references, such as Mullis, et al. (1987) U.S. Pat. No. 4,683,202; Arnheim & Levinson (1990) C&EN 68:36-47; Kwoh et al. (1989) Proc Natl Acad Sci USA 86:1173; Guatelli et al. (1990) Proc Natl Acad Sci USA 87:1874; Lomeli et al. (1989) J Clin Chem 35:1826; Landegren et al. (1988) Science 241:1077-1080; Van Brunt (1990) Biotechnology 8:291-294; Wu & Wallace (1989) Gene 4:560; Barringer et al. (1990) Gene 89:117; and Sooknanan & Malek (1995) Biotechnology 13:563-564.

Such nucleic acid amplification techniques can be applied to amplify and/or detect nucleic acids of interest, such as nucleic acids comprising marker loci. Amplification primers for amplifying useful marker loci and suitable probes to detect useful marker loci or to genotype alleles, such as SNP alleles, are provided. However, one of skill will immediately recognize that other primer and probe sequences could also be used. For instance primers to either side of the given primers can be used in place of the given primers, so long as the primers can amplify a region that includes the allele to be detected, as can primers and probes directed to other marker loci. Further, it will be appreciated that the precise probe to be used for detection can vary, e.g., any probe that can identify the region of a marker amplicon to be detected can be substituted for those examples provided herein, and the configuration of the amplification primers and detection probes can, of course, vary. Thus, the compositions and methods are not limited to the primers and probes specifically recited herein.

In certain examples, probes will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. A probe can also constitute radiolabelled PCR primers that are used to generate a radiolabelled amplicon. Labeling strategies for nucleic acids and corresponding detection strategies can be found, e.g., in Haugland (1996) Handbook of Fluorescent Probes and Research Chemicals (6th Ed.), Molecular Probes, Inc. (Eugene, Oreg.); or in Haugland (2001) Handbook of Fluorescent Probes and Research Chemicals (8th Ed.), Molecular Probes, Inc. (Eugene, Oreg.).

Detectable labels may also include reporter-quencher pairs, such as are employed in Molecular Beacon and TaqMan™ probes. The reporter may be a fluorescent organic dye modified with a suitable linking group for attachment to the oligonucleotide, such as to the terminal 3′ carbon or terminal 5′ carbon. The quencher may also be an organic dye, which may or may not be fluorescent. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules (2nd Ed.), Academic Press, New York, 1971, the content of which is incorporated herein by reference. Examples of modifying reporters and quenchers for covalent attachment via common reactive groups that can be added to an oligonucleotide in the present invention may be found, for example, in Haugland (2001) Handbook of Fluorescent Probes and Research Chemicals (8th Ed.), Molecular Probes, Inc. (Eugene, Oreg.), the content of which is incorporated herein by reference.

In certain examples, reporter-quencher pairs are selected from xanthene dyes including fluorescein and rhodamine dyes. Many suitable forms of these compounds are available commercially with substituents on the phenyl groups, which can be used as the site for bonding or as the bonding functionality for attachment to an oligonucleotide. Another useful group of fluorescent compounds for use as reporters are the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like. In certain other examples, the reporters and quenchers are selected from fluorescein and rhodamine dyes. These dyes and appropriate linking methodologies for attachment to oligonucleotides are well known in the art.

Suitable examples of reporters may be selected from dyes such as SYBR green, 5-carboxyfluorescein (5-FAM™ available from Applied Biosystems (Foster City, Calif., USA), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from available from Applied Biosystems, and the like. Suitable examples of quenchers may be selected from 6-carboxy-tetramethyl-rhodamine, 4-(4-dimethylaminophenylazo)benzoic acid (DABYL), tetramethylrhodamine (TAMRA), BHQ-0™, BHQ-1™, BHQ-2™, and BHQ-3™, each of which are available from Biosearch Technologies, Inc. (Novato, Calif., USA), QSY-7™, QSY-9™, QSY-21™ and QSY-35™, each of which are available from Molecular Probes, Inc., and the like.

In one aspect, real time PCR or LCR is performed on the amplification mixtures described herein, e.g., using molecular beacons or TaqMan™ probes. A molecular beacon (MB) is an oligonucleotide which, under appropriate hybridization conditions, self-hybridizes to form a stem and loop structure. The MB has a label and a quencher at the termini of the oligonucleotide; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, such as to a region of an amplicon during amplification), the MB label is unquenched. Details regarding standard methods of making and using MBs are well established in the literature and MBs are available from a number of commercial reagent sources. See also, e.g., Leone, et al., (1995) Nucl Acids Res 26:2150-2155; Tyagi & Kramer (1996) Nature Biotechnol 14:303-308; Blok & Kramer (1997) Mol Cell Probes 11:187-194; Hsuih et al. (1997) J Clin Microbiol 34:501-507; Kostrikis et al. (1998) Science 279:1228-1229; Sokol et al. (1998) Proc Natl Acad Sci USA 95:11538-11543; Tyagi et al. (1998) Nature Biotechnol 16:49-53; Bonnet et al. (1999) Proc Natl Acad Sci USA 96:6171-6176; Fang et al. (1999) J Am Chem Soc 121:2921-2922; Marras et al. (1999) Genet Anal Biomol Eng 14:151-156; and, Vet et al. (1999) Proc Natl Acad Sci USA 96:6394-6399. Additional details regarding MB construction and use are also found in the patent literature, e.g., U.S. Pat. Nos. 5,925,517; 6,150,097; and 6,037,130.

Another real-time detection method is the 5′-exonuclease detection method, also called the TaqMan™ assay, for example as set forth in U.S. Pat. Nos. 5,804,375; 5,538,848; 5,487,972; and 5,210,015, each of which is hereby incorporated by reference in its entirety. In the TaqMan™ assay, a modified probe, typically 10-30 nucleotides in length, is employed during PCR which binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′ to 3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are typically attached to the probe within a few nucleotides of one another, usually within 30 nucleotides of one another, or within 6 to 16 nucleotides. Typically, this separation is achieved by attaching one member of a reporter-quencher pair to the 5′ end of the probe and the other member to a nucleotide about 6 to 16 nucleotides away, in some cases at the 3′ end of the probe.

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

One example of a suitable real-time detection technique that does not use a separate probe that binds intermediate to the two primers is the KASPar detection system/method, which is well-known in the art. In KASPar, two allele specific primers are designed such that the 3′ nucleotide of each primer hybridizes to the polymorphic base. For example, if the SNP is an A/C polymorphism, one of the primers would have an “A” in the 3′ position, while the other primer would have a “C” in the 3′ position. Each of these two allele specific primers also has a unique tail sequence on the 5′ end of the primer. A common reverse primer is employed that amplifies in conjunction with either of the two allele specific primers. Two 5′ fluor-labeled reporter oligos are also included in the reaction mix, one designed to interact with each of the unique tail sequences of the allele-specific primers. Lastly, one quencher oligo is included for each of the two reporter oligos, the quencher oligo being complementary to the reporter oligo and being able to quench the fluor signal when bound to the reporter oligo. During PCR, the allele-specific primers and reverse primers bind to complementary DNA, allowing amplification of the amplicon to take place. During a subsequent cycle, a complementary nucleic acid strand containing a sequence complementary to the unique tail sequence of the allele-specific primer is created. In a further cycle, the reporter oligo interacts with this complementary tail sequence, acting as a labeled primer. Thus, the product created from this cycle of PCR is a fluorescently-labeled nucleic acid strand. Because the label incorporated into this amplification product is specific to the allele specific primer that resulted in the amplification, detecting the specific fluor presenting a signal can be used to determine the SNP allele that was present in the sample.

Further, it will be appreciated that amplification is not a requirement for marker detection, for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established and are taught, e.g., in Sambrook et al. Molecular Cloning—A Laboratory Manual (3d ed.) Vol. 1-3, Cold Spring Harbor Laboratory, Cold Spring Harbor, N.Y., 2000 (“Sambrook”); Current Protocols in Molecular Biology, F.M. Ausubel et al., eds., Current Protocols, a joint venture between Greene Publishing Associates, Inc. and John Wiley & Sons, Inc., (supplemented through 2002) (“Ausubel”); and, PCR Protocols A Guide to Methods and Applications (Innis et al., eds) Academic Press Inc. San Diego, Calif. (1990) (“Innis”), additional details regarding detection of nucleic acids in plants can also be found, e.g., in Plant Molecular Biology (1993) Croy (ed.) BIOS Scientific Publishers, Inc., each of these are herein incorporated by reference in their entirety.

Other techniques for detecting SNPs can also be employed, such as allele specific hybridization (ASH) or nucleic acid sequencing techniques. ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

Real-time amplification assays, including MB or TaqMan™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 13-18 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end. By detecting signal for each label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

Introgression of herbicide tolerance into less tolerant soybean germplasm is provided. Any method for introgressing a QTL or marker into soybean plants known to one of skill in the art can be used. Typically, a first soybean germplasm having tolerance to at least one herbicide based on a particular marker or haplotype and a second soybean germplasm that lacks such tolerance are provided. The first soybean germplasm may be crossed with the second soybean germplasm to provide progeny soybean germplasm. These progeny germplasm are screened to determine the presence of herbicide tolerance derived from the marker or haplotype, and progeny that tests positive for the presence of tolerance derived from the marker or haplotype are selected as being soybean germplasm into which the marker or haplotype has been introgressed. Methods for performing such screening are well known in the art and any suitable method can be used.

One application of MAS is to use the tolerance markers or haplotypes to increase the efficiency of an introgression or backcrossing effort aimed at introducing a tolerance trait into a desired (typically high yielding) background. In marker assisted backcrossing of specific markers from a donor source, e.g., to an elite genetic background, one selects among backcross progeny for the donor trait and then uses repeated backcrossing to the elite line to reconstitute as much of the elite background's genome as possible.

Thus, the markers and methods can be utilized to guide marker assisted selection or breeding of soybean varieties with the desired complement (set) of allelic forms of chromosome segments associated with superior agronomic performance (tolerance, along with any other available markers for yield, disease tolerance, etc.). Any of the disclosed marker alleles or haplotypes can be introduced into a soybean line via introgression, by traditional breeding (or introduced via transformation, or both) to yield a soybean plant with superior agronomic performance. The number of alleles associated with tolerance that can be introduced or be present in a soybean plant ranges from 1 to the number of alleles disclosed herein, each integer of which is incorporated herein as if explicitly recited.

This also provides a method of making a progeny soybean plant and these progeny soybean plants, per se. The method comprises crossing a first parent soybean plant with a second soybean plant and growing the female soybean plant under plant growth conditions to yield soybean plant progeny. Methods of crossing and growing soybean plants are well within the ability of those of ordinary skill in the art. Such soybean plant progeny can be assayed for alleles associated with tolerance and, thereby, the desired progeny selected. Such progeny plants or seed can be sold commercially for soybean production, used for food, processed to obtain a desired constituent of the soybean, or further utilized in subsequent rounds of breeding. At least one of the first or second soybean plants is a soybean plant that comprises at least one of the markers or haplotypes associated with tolerance, such that the progeny are capable of inheriting the marker or haplotype.

Often, a method is applied to at least one related soybean plant such as from progenitor or descendant lines in the subject soybean plants pedigree such that inheritance of the desired tolerance can be traced. The number of generations separating the soybean plants being subject to the methods will generally be from 1 to 20, commonly 1 to 5, and typically 1, 2, or 3 generations of separation, and quite often a direct descendant or parent of the soybean plant will be subject to the method (i.e., 1 generation of separation).

Genetic diversity is important for long term genetic gain in any breeding program. With limited diversity, genetic gain will eventually plateau when all of the favorable alleles have been fixed within the elite population. One objective is to incorporate diversity into an elite pool without losing the genetic gain that has already been made and with the minimum possible investment. MAS provides an indication of which genomic regions and which favorable alleles from the original ancestors have been selected for and conserved over time, facilitating efforts to incorporate favorable variation from exotic germplasm sources (parents that are unrelated to the elite gene pool) in the hopes of finding favorable alleles that do not currently exist in the elite gene pool. For example, the markers, haplotypes, primers, and probes can be used for MAS involving crosses of non-elite lines to elite lines or exotic lines, elite lines to exotic soybean lines (elite X exotic), or any other crossing strategy, by subjecting the segregating progeny to MAS to maintain major yield alleles, along with the tolerance marker alleles herein.

As an alternative to standard breeding methods of introducing traits of interest into soybean (e.g., introgression), transgenic approaches can also be used to create transgenic plants with the desired traits. In these methods, exogenous nucleic acids that encode a desired QTL, marker, or haplotype are introduced into target plants or germplasm. For example, a nucleic acid that codes for a ALS-inhibiting herbicide tolerance trait is cloned, e.g., via positional cloning, and introduced into a target plant or germplasm.

Experienced plant breeders can recognize herbicide tolerant soybean plants in the field, and can select the tolerant individuals or populations for breeding purposes or for propagation. In this context, the plant breeder recognizes “tolerant” and “non-tolerant” or “susceptible” soybean plants. However, plant tolerance is a phenotypic spectrum consisting of extremes in tolerance and susceptibility, as well as a continuum of intermediate tolerance phenotypes. Evaluation of these intermediate phenotypes using reproducible assays are of value to scientists who seek to identify genetic loci that impart tolerance, to conduct marker assisted selection for tolerant populations, and to use introgression techniques to breed a tolerance trait into an elite soybean line, for example.

Phenotypic screening and selection of tolerant and/or susceptible soybean plants may be performed, for example, by exposing plants to a herbicidal compound comprising at least one ALS-inhibiting herbicide, including but not limited to examples such as spray tests, dosage tests, leaf painting assays, tissue culture assays, and/or germination assays, and selecting those plants showing tolerance. Any such assay known to the art may be used, e.g., as described in Odell et al. (1990) Plant Physiol 94:1647-1654, U.S. Pat. No. 5,084,082, and Sebastian et al. (1989) Crop Sci 29:1403-1408, each of which is incorporated herein by reference in its entirety, or as described herein.

In some examples, a kit or an automated system for detecting markers or haplotypes, and/or for correlating the markers or haplotypes with a desired phenotype (e.g., ALS-inhibiting herbicide tolerance), are provided. Thus, a typical kit can include a set of marker probes and/or primers configured to detect at least one favorable allele of one or more marker locus associated with tolerance, improved tolerance, or susceptibility to ALS-inhibiting herbicides. These probes or primers can be configured, for example, to detect the marker alleles noted in the tables and examples herein, e.g., using any available allele detection format, such as solid or liquid phase array based detection, microfluidic-based sample detection, etc. The kits can further include packaging materials for packaging the probes, or primers, instructions, controls, such as control amplification reactions that include probes, primers, and/or template nucleic acids for amplifications, molecular size markers, buffers, other reagents, containers for mixing and/or reactions, or the like.

A typical system can also include a detector that is configured to detect one or more signal outputs from the set of marker probes or primers, or amplicon thereof, thereby identifying the presence or absence of the allele. A wide variety of signal detection apparatus are available, including photo multiplier tubes, spectrophotometers, CCD arrays, scanning detectors, phototubes and photodiodes, microscope stations, galvo-scans, microfluidic nucleic acid amplification detection appliances, and the like. The precise configuration of the detector will depend, in part, on the type of label used to detect the marker allele, as well as the instrumentation that is most conveniently obtained for the user. Detectors that detect fluorescence, phosphorescence, radioactivity, pH, charge, absorbance, luminescence, temperature, magnetism or the like can be used.

Typical detector examples include light (e.g., fluorescence) detectors or radioactivity detectors. For example, detection of a light emission (e.g., a fluorescence emission) or other probe label is indicative of the presence or absence of a marker allele. Fluorescent detection is generally used for detection of amplified nucleic acids (however, upstream and/or downstream operations can also be performed on amplicons, which can involve other detection methods). In general, the detector detects one or more label (e.g., light) emission from a probe label, which is indicative of the presence or absence of a marker allele. The detector(s) optionally monitors one or a plurality of signals from an amplification reaction. For example, the detector can monitor optical signals which correspond to “real time” amplification assay results.

System or kit instructions that describe how to use the system or kit and/or that correlate the presence or absence of the allele with the predicted tolerance or susceptibility phenotype are also provided. For example, the instructions can include at least one look-up table that includes a correlation between the presence or absence of the favorable allele(s) and the predicted tolerance or improved tolerance. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector.

Dominant sulfonylurea herbicide resistance could be used to produce experimental or commercial quantities of pure F1 hybrid seeds. In one example, a herbicide-resistant line that is rendered male sterile through genetic, chemical, and/or manual means can be planted, either interplanted or in separate rows, in the same field with a male fertile but herbicide sensitive line. After pollination, the male parent can be removed from the field with an ALS-inhibiting herbicide, for example a sulfonylurea herbicide treatment that is selectively lethal to the male parent. The entire field (containing F1 seeds borne by the resistant female line) can then be bulk harvested without seed contamination from the male line. Dominant sulfonylurea resistance could also be used for experimental or commercial production of F2 varieties. In one example, F1 hybrid seed would be produced on a herbicide sensitive female parent (rendered at least partially male sterile) that is fertilized by pollen from a parent with dominant homozygous herbicide resistance. Male and female parents could be planted in separated rows to facilitate mechanical harvest of seed from the female parent. Resulting F1 seeds/plants will be heterozygous for herbicide resistance, undesirable seeds/plants resulting from self-pollination of the female parent can be rogued from a population of F1 seeds/plants with a herbicide treatment that is selectively lethal to the sensitive female line to produce a pure stand of F1 plants that could be bulk harvested for the production of a pure F2 seed population.

A selectively lethal sulfonylurea treatment could be used to rogue sensitive plants from sulfonylurea-resistant populations that have been contaminated through seed handling operations. The use of other dominant markers (such as purple hypocotyls and tawny pubescence) requires visual inspection of each plant for expression of the marker and hand removal of undesirable types. Such labor makes these visual markers less practical for commercial-scale purification of inbred lines or hybrids. With dominant herbicide resistance, large seed production fields can be easily eliminated by spraying the entire field with an herbicide treatment that is lethal to herbicide-sensitive plants. Isolated nucleic acids comprising a nucleic acid sequence coding for tolerance or susceptibility to at least one ALS-inhibiting herbicide, or capable of detecting such a phenotypic trait, or sequences complementary thereto, are also included. In certain examples, the isolated nucleic acids are capable of hybridizing under stringent conditions to nucleic acids of a soybean cultivar displaying tolerance to at least one ALS-inhibiting herbicide, for instance to particular markers, including but not limited to one or more of S12761-1 on linkage group C1, a marker locus comprising nucleotides 43645228-43645929 of Glyma04g37270.1, a marker that detects a polymorphism at nucleotide 43645620 of Glyma04g37270.1, a marker that detects a polymorphism that encodes a P178S mutation in an acetolactate synthase gene on chromosome 4; S12764-1 on linkage group C2; a marker locus comprising nucleotides 14143182-14143881 of Glyma06g17790.1, a marker that detects a polymorphism at nucleotide 14143499 of Glyma06g17790.1, a marker that detects a polymorphism that encodes a W560L mutation in an acetolactate synthase gene on chromosome 6, a marker locus closely linked to any of the marker loci, and any combination of thereof.

Vectors comprising such nucleic acids, expression products of such vectors expressed in a host compatible therewith, antibodies to the expression product (both polyclonal and monoclonal), and antisense nucleic acids are also included.

Soybean plants and germplasm disclosed herein or derived therefrom or identified using the methods provided and having marker loci associated with ALS-herbicide tolerance may be used as a parental line. Also included are soybean plants produced by any of the foregoing methods. Seed of a soybean germplasm produced by crossing a soybean variety having a marker, a haplotype, and/or a marker profile associated with ALS-herbicide tolerance with a soybean variety lacking such marker, haplotype, and/or marker profile and progeny thereof, is also included.

The present invention is illustrated by the following examples. The foregoing and following description of the present invention and the various examples are not intended to be limiting of the invention but rather are illustrative thereof. Hence, it will be understood that the invention is not limited to the specific details of these examples.

### EXAMPLES

### Example 1

### ALS Mutants

Soybean plants comprising mutations in an acetolactate synthase (ALS) gene were produced, selected, and characterized as described in U.S. Pat. No. 5,084,082 and Sebastian et al. (1989) Crop Sci 29:1403-1408. Briefly, in order to produce soybean lines with tolerance or higher tolerance to sulfonylurea-containing herbicides (SUs), seed from 3 parental cultivars were chemically mutagenized under various mutagen, mutagen concentration, and/or exposure experimental regimes. Approximately 50,000 seed (8.6 kg) from SU-sensitive parental cultivars Williams, Williams82, and A3205 were treated with the mutagens ethyl methane sulfonate (EMS) or n-nitroso-N-methylurea (NMU). Treated seed were planted to produce M1 plants which progressed through maturity to produce M2 seed. M2 seed was harvested in bulk from each M1 plant, in total eight different M2 populations were produced. From these efforts, an SU tolerant line denoted as W20 was produced from the Williams cultivar. Extensive testing indicated that the W20 phenotype is based on a semidominant SU-resistance allele in an ALS gene, denoted as Als1. It was hypothesized that it was highly probable that W20 and five other mutants in the same population could have been derived from the same M1 source event. Further testing did not disprove this hypothesis, and therefore W20 was considered to represent a class of mutants which also included line W4.

Once true-breeding herbicide resistant mutant lines were established after one round of treatment, a seed increase was done to obtain starting material for a second round of mutagenesis with NMU. The resulting M2 populations were screened by exposure to a sulfonylurea herbicide treatment which was lethal to the starting material. This process identified a second generation mutant denoted as W4-4 comprising greater herbicide tolerance than parental line W4. W4-4 was allowed to self-pollinate to produce a true-breeding line.

### Example 2

### DNA Isolation and Sequencing

W4-4 soybean seeds were sterilized in 95% ethanol for 1 minute, 15% v/v bleach for 5 minutes, followed by 3 washes with sterile water. Five seeds were sandwiched between two sheets of sterile paper (Whatman Cat #1001 125) moistened with deionized water and placed in a 120 mm ½″ plate (Falcon #351058), then sealed with PARAFILM™. The plate of seeds was kept in a growth chamber at 26° C. for four days and allowed to germinate.

Two seeds germinated and had roots approximately 0.5 cm long that were removed. Half of each seed was added to a 2-ml tube along with two 5/32″ stainless steel balls. The tubes were placed in a GenoGrinder 2000 to grind the tissue with settings 1×250 strokes/min for 1.5 minutes. The tubes were spun 1 minute at 13,000 rcf in a microcentrifuge. The grind and spin were repeated once more. Genomic DNA was then isolated at room temperature using the Qiagen DNAeasy Plant Mini Kit (Cat. No. 69104), as per the manufacturer's protocol.

The cDNA sequence for the Als1 catalytic subunit was used to search a proprietary sequence database (UniSoy 3.0) using the blastn program (Altschul et al. (1997) Nucl Acids Res 25:3389-3402). Nine genes described as acetolactate synthase sequences were identified and mapped to the soy genome using a proprietary genome browser tool to utilize the JGI/DOE soy genomic sequence assembly Glymal (Schmutz et al. (2010) Nature 463:178-183) for wild type soybean genotype Williams 82. These nine genes corresponded to five loci annotated as ALS catalytic subunit genes.

| Gene ID | Fgenesh | ORF (AA) | Note | Physical Position |
|---|---|---|---|---|
| Glyma01g09920.1 | GM01_5367 | 278 | | |
| Glyma04g37270.1 | GM04_21145 | 641 | Als1 | 43645228-43645929 |
| Glyma06g17790.1 | GM06_4197 | 645 | Als2 | 14143182-14143881 |
| Glyma13g31470.1 | GM13_13846 | 645 | | |
| Glyma15g07860.1 | GM15_1662 | 653 | | |

PCR primers were designed to flank the genes predicted by the Fgenesh algorithm at these loci:

| (SEQ ID NO. 20) | |
|---|---|
| GM01F | GAAACTCTCCACCGCCTC |
| (SEQ ID NO. 21) | |
| GM01R | GATCACTAAGTAACCATTAAAGAC |
| (SEQ ID NO. 22) | |
| GM04F | TTAATAAATTTTCTACATCCCAGTGA |
| (SEQ ID NO. 23) | |
| GM04R | GATGCTACTGCATGTAGTAAG |
| (SEQ ID NO. 24 | |
| GM06F | GACACACTCTGAGAGTCTC |
| (SEQ ID NO. 25) | |
| GM06R | TACCAAAACTACTGCAAACTATG |
| (SEQ ID NO. 26) | |
| GM13F | ACCTAAGTTAATTCATGAAATGTTTG |
| (SEQ ID NO. 27) | |
| GM13R | GCTATATTAGCTTACTATTTTTACAAAAC |
| (SEQ ID NO. 28) | |
| GM15F | GATCATTAAACGTTTTAACGCG |
| (SEQ ID NO. 29) | |
| GM15R | TATCTTAGTTGCCAACATGAATAC |

Using W4-4 genomic DNA as the template, PCR products for the ALS genes were cloned using Finnzymes Phusion DNA polymerase (Cat. No. F-530S), following the instructions in the manufacturer's protocol. The PCR products were cloned into pCR-BluntII-TOPO and transformed into TOP10 cells (Invitrogen, Cat. No. K2800-20). Sequencing was done by Sequetech (Mountain View, Calif.). Sequences were aligned with Sequencher 4.8 software.

Two 6-mm leaf punches per tube were taken from the first trifoliate leaves of 4-week old W4-4 plants and stored at −80° C. The Qiagen RNeasy Mini Plant Kit (Cat. No. 74904) was used for the purification of total RNA and a GenoGrinder 2000 was used to grind the tissue with 2 steel 3/32″ balls and buffer RLT with β-mercaptoethanol. The tissue was ground using GenoGrinder set at 1×250 strokes/minute for 1.5 minutes. Synthesis of cDNA was done with Invitrogen's 3′ RACE Kit (Cat. No. 18373019) using 1 μg total RNA as template, and included a no-reverse-transcriptase control. Finnzymes Phusion DNA polymerase (Cat. No. F-530S) was used for PCR with the cDNA as starting template, following the manufacturer's protocol. PCR forward primers were designed in the 5′ untranslated regions on chromosomes 4 and 6, and reverse primers located in the gene near the stop codon and in the terminator region.

| (SEQ ID NO. 30) | |
|---|---|
| ALS1F | TGGTGCTACCCACACAACAC |
| (SEQ ID NO. 31) | |
| ALS2F | CAGTGCAGCCACACAAAGAC |
| (SEQ ID NO. 32) | |
| ALS3′UTRR | CTCACCACAGGCCAAATC |
| (SEQ ID NO: 33) | |
| ALSR | CATCCTTGAAGGATCCATTACTGGGAATCA |

To confirm that the Als1 and Als2 expression results from cDNA cloning, reverse transcription quantitative PCR (RT-qPCR) was performed. Approximately 6 μg of total RNA isolated as described above was DNase treated with Qiagen RNase-Free DNase Set (Cat No. 79254) following the manufacturer's RNA Cleanup protocol with DNase on-column digestion in the Qiagen RNeasy Mini Kit (Cat. No. 74104). Synthesis of cDNA was performed using Invitrogen's 3′ RACE Kit (Cat. No. 18373019) using 870 ng of total RNA as template, and including a no-reverse-transcriptase control. The cDNA and the no-reverse-transcriptase control were both diluted with 60 μl of TE. Forward and reverse primers were used to amplify regions of approximately 200 bp long for Als1 and Als2. Primers to amplify a region in the EF1a gene were used as a control. RT-qPCR primer sequences:

| (SEQ ID NO: 34) | |
|---|---|
| ALS1QF | CTTCACCAAGGAAGCGC |
| (SEQ ID NO. 35) | |
| ALS1QR | TTCGGCGGCGAAGAC |
| (SEQ ID NO. 36) | |
| ALS2QF | CGCCGGCAACATCAG |
| (SEQ ID NO. 37) | |
| ALS2QR | TCGGCGGCGAAGATG |
| (SEQ ID NO. 38) | |
| EF1AQF | ATGCTGCGCAGACAGTCACT |
| (SEQ ID NO. 39) | |
| EF1AQR | CAGCCTCATCCAATACAAACATCT |

PerfeCTa SYBR Green Supermix, UNG (Quanta Biosciences 95068-500) was used for each reaction, which was scaled down to 25 μl from the manufacturer's original 50 μl protocol, as well as 2 μl of diluted cDNA (or no-RT control) and 1.25 μl of each 10 μM primer. Each template/primer pair reaction was done in triplicate. Amplification was performed with a Bio-Rad Chromo4 Real-Time Detector with a DNA Engine thermal cycler. Initial incubation was at 50° C. for 2 minutes, followed by denaturation at 95° C. for 2 minutes. 40 PCR cycles were performed: denaturation at 95° C. for 15 seconds, annealing at 58° C. for 10 seconds, and elongation at 72° C. for 15 seconds. This was followed by a melting curve program reading every 0.2° C. from 65° C. to 85° C. Opticon Monitor 3.1.32 software was used to analyze the results.

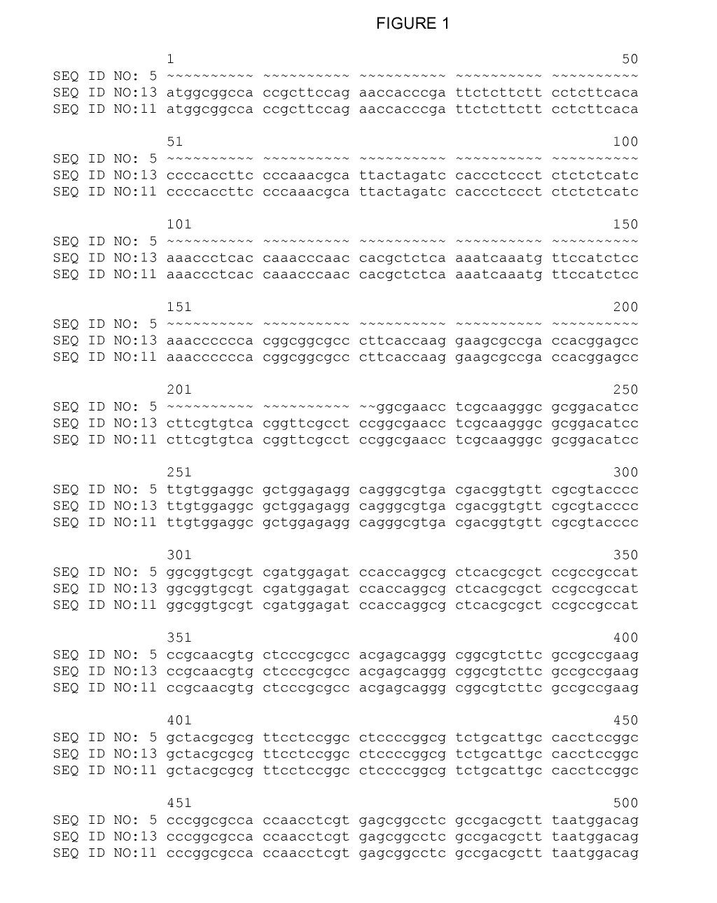

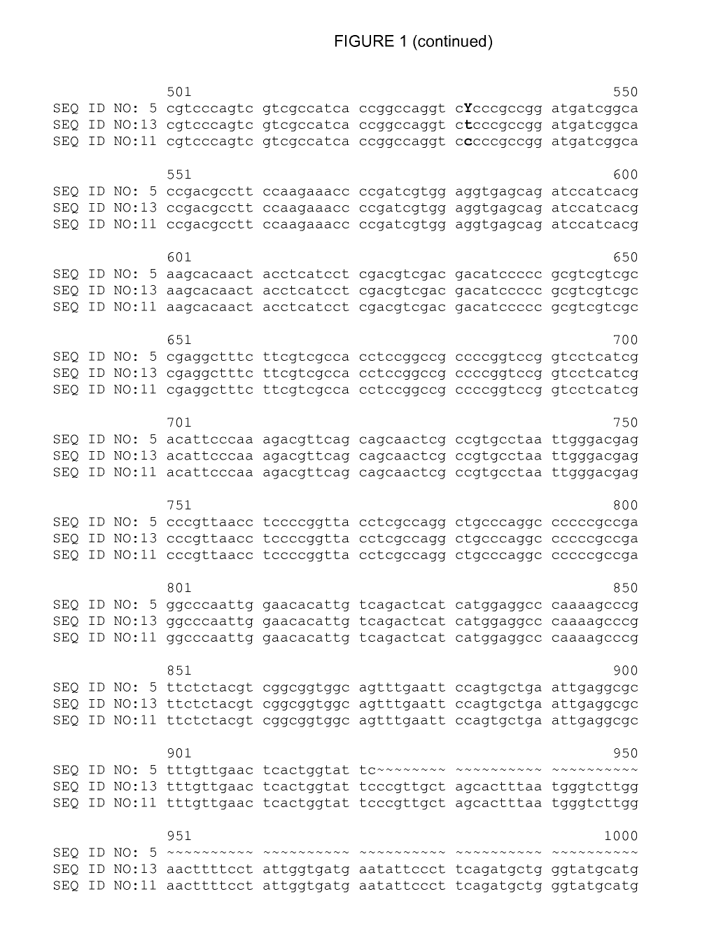

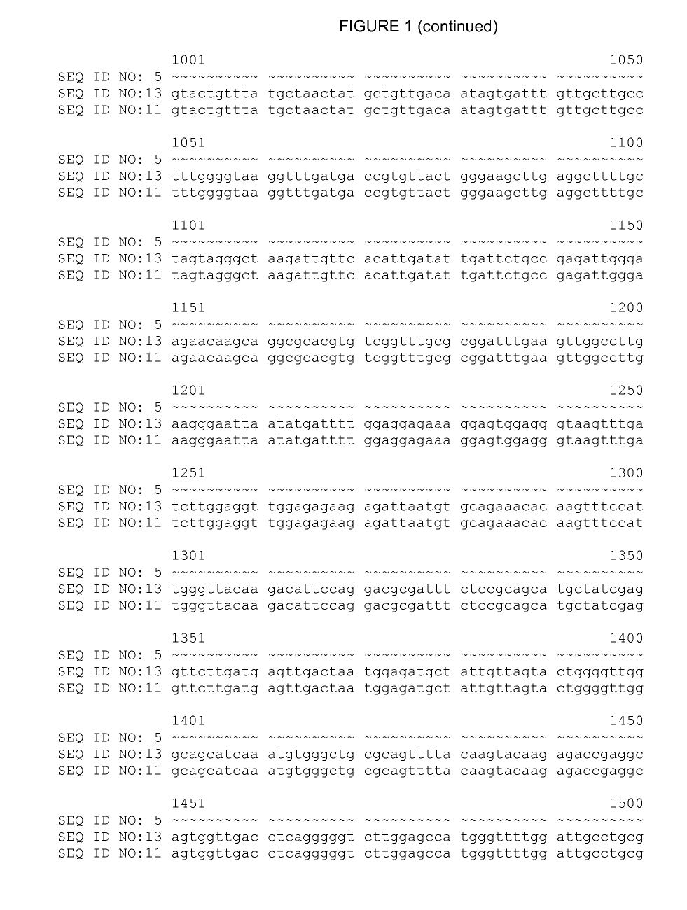

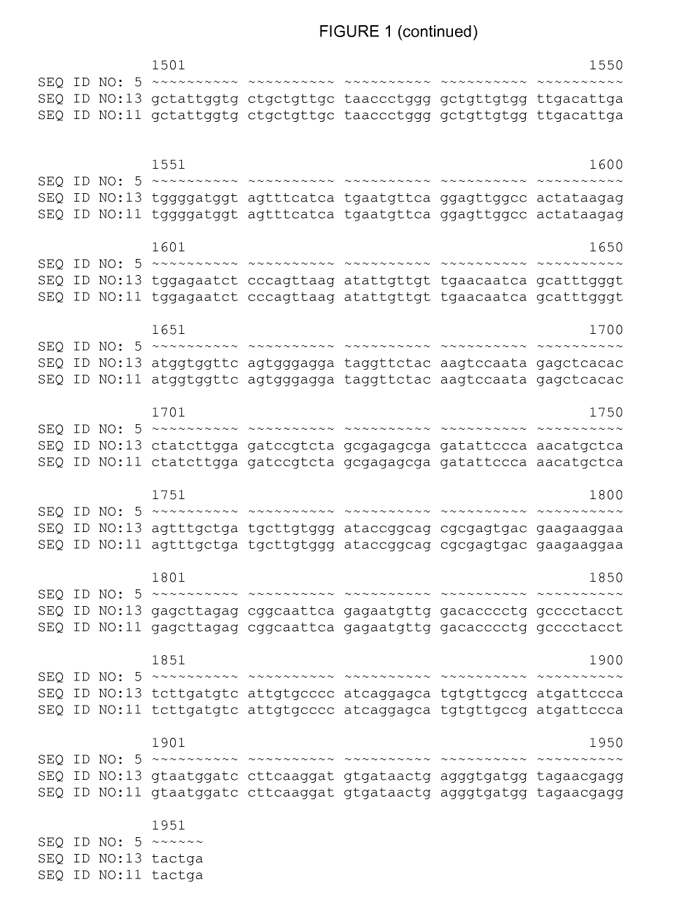

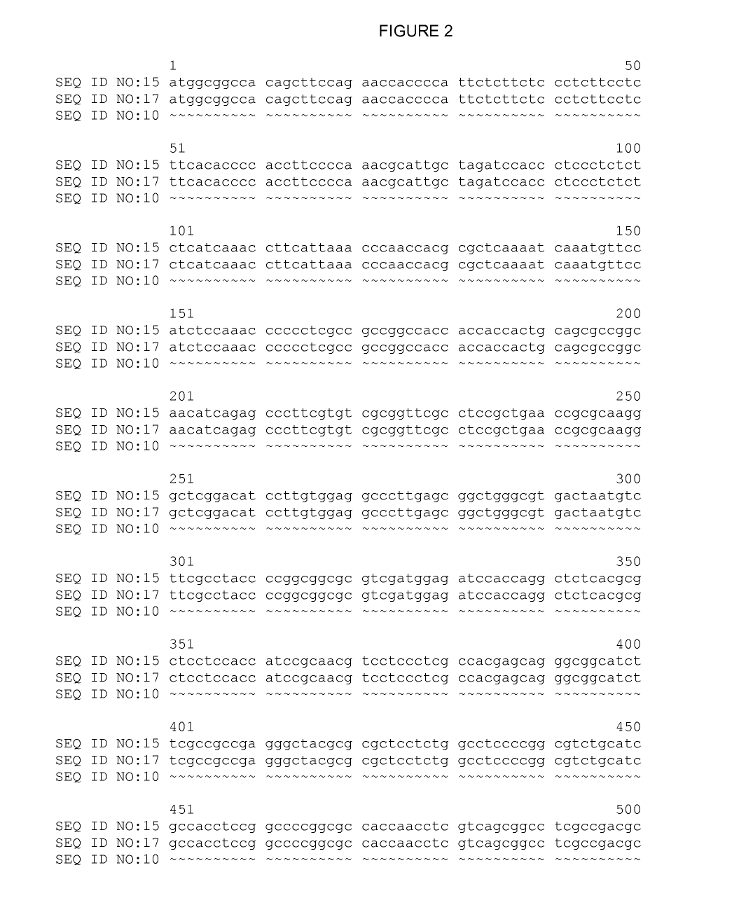

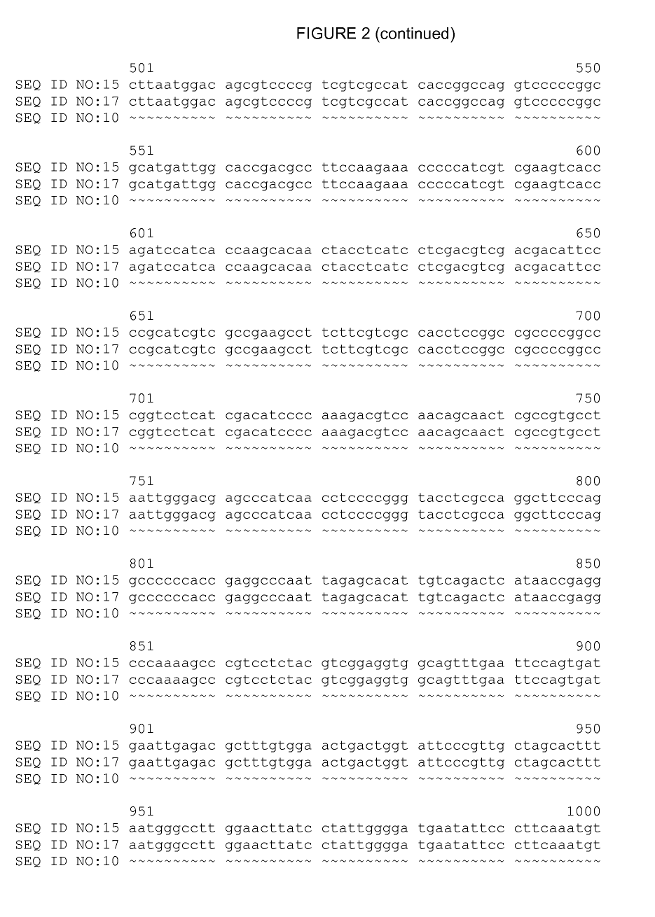

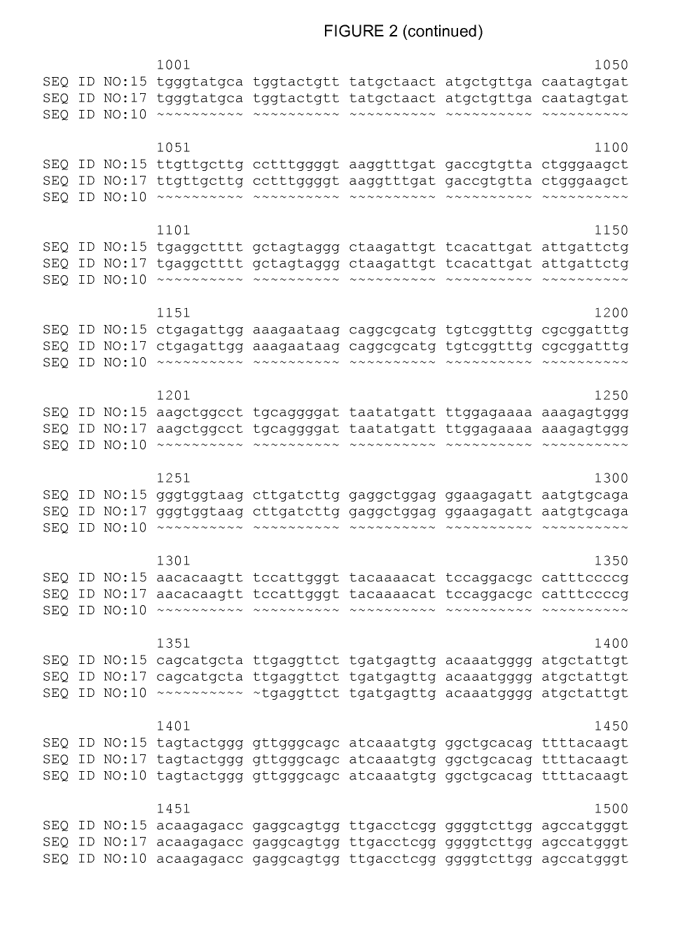

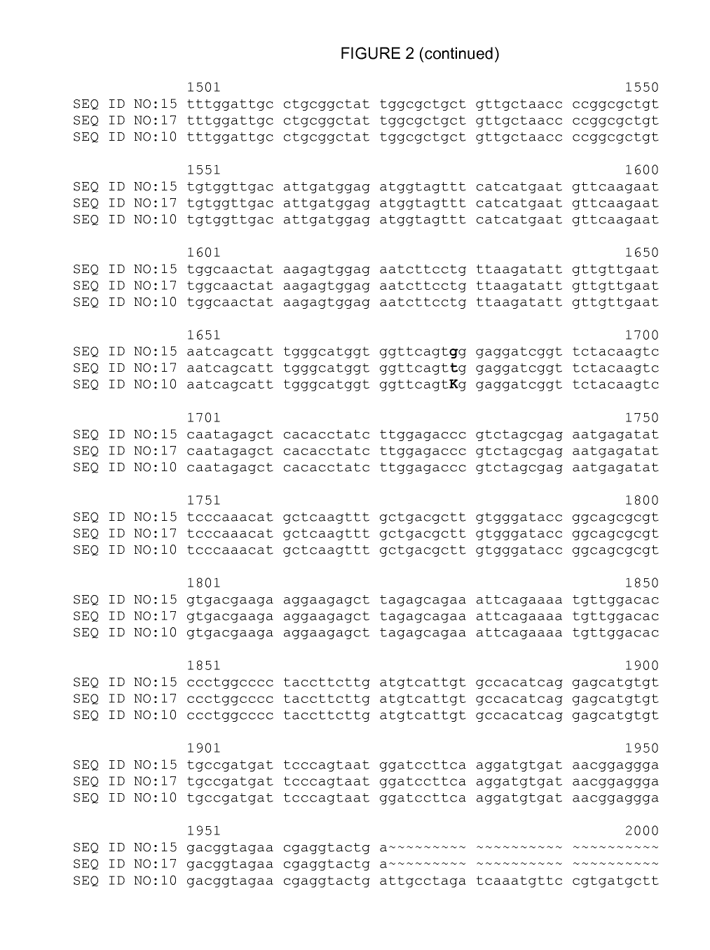

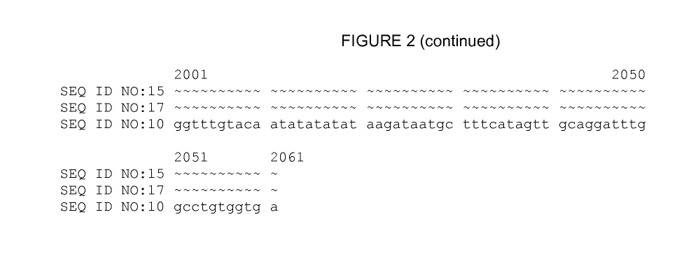

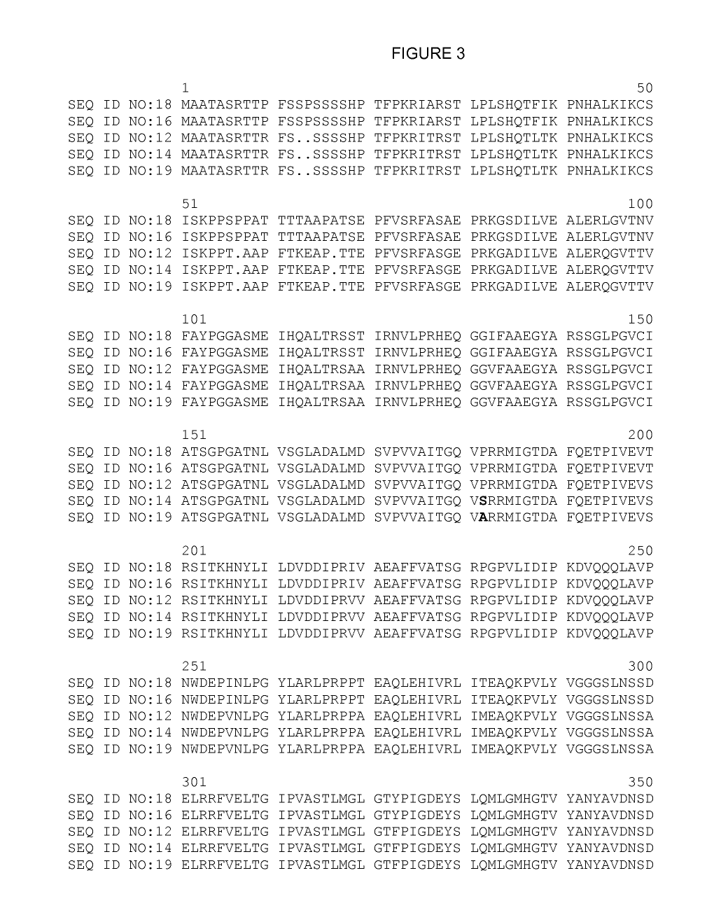

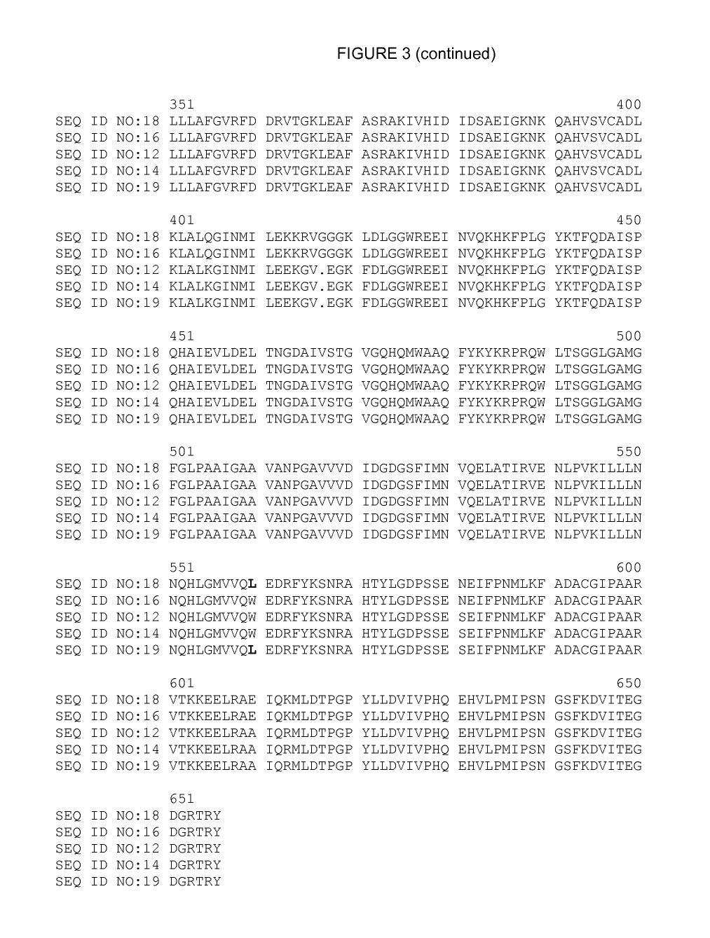

Sequencing results for the five ALS genes showed a mutation in 2 out of the 5 genes identified by BLASTN, Glyma04g37270.1 and Glyma06g17790.1. The gene in Glyma04g37270.1 corresponds to the Als1 designated in Sebastian et al. (1989) Crop Sci 291403-1408. The gene in Glyma06g17790.1 is herein designated as Als2. Alignments of wild type als1 and als2 alleles with their W4-4 counterparts were made with the AlignX feature in Vector NTI software (Invitrogen). Mutation of Als1 allele from the W4-4 line resulted in a proline to serine substitution at position 178. Mutation of Als2 allele in the W4-4 line resulted in a tryptophan to leucine substitution at position 560. These mutations are similar to the mutations in the highly herbicide-resistant ALS variant known as HRA, which has mutations P178A and W555L on the same (Als1) gene (U.S. Pat. Nos. 5,013,659, 5,141,870, 5,378,824, 5,605.011; Lee et al. (1988) EMBO J. 7:1241-1248; Mazur & Falco (1989) Ann Rev Plant Biol 40:441-470). Amino acid position 555 in soybean Als1 is analogous to amino acid position 560 in the soybean Als2 (see, e.g., FIG. 3). Multiple sequence alignments were generated using PileUp (GCG SeqWeb, Accelrys) and are shown in FIGS. 1-3, variant nucleotides and amino acids are shown in bold.

### Example 3

### Genetic Markers for Mutant ALS Sequences

The genetic sequence information confirms that the Als1 and Als2 mutations are caused by base substitutions within the coding regions of soybean ALS genes. These results also confirm previous evidence that Als1 and Als2 are unlinked and on different chromosomes (GM04 and GM06, respectively). The gene sequences and position(s) of the exact sequence changes were used to develop genetic markers for detection of the causative SNPs responsible for the herbicide-resistant phenotypes. Markers designed around causative SNPs are preferred to markers that are merely linked with the trait of interest. For example, even closely-linked markers could be linked in repulsion in some germplasm, or could become decoupled from the causative SNP due to recombination in future breeding cycles. Optimized markers facilitate rapid and precise incorporation of these commercially-useful mutations into a wide variety of elite soybean germplasm.

Using the sequence information determined in Example 2, marker primers and probes specific for als1 and als2 wildtype alleles (Williams82) and Als1 and Als2 W4-4 alleles were designed using several software tools including Pioneer proprietary software, Primer Express (Applied Biosystems, Foster City, Calif., USA), Primer 3 (open source community-development project hosted by SourceForge), and Primer Prim'r (Everett et al. (2004) J Structural Functional Genomics 5:13-21).

### Marker Alleles in Tolerant and Susceptible Lines

| | S12761 | S12764 |
|---|---|---|
| | ALS1 Locus | ALS2 locus |
| Tolerant allele | T (Als1) | T (Als2) |
| Susceptible allele | C (als1) | G (als2) |
| Physical position | 43645620 | 14143499 |

Designed markers were validated using W4-4, Williams82, and a panel of 11 proprietary Pioneer varieties adapted to production in southern environments. Thirty-two seed for each of W4-4 and Williams82 were planted in a growth chamber, 25 of which germinated for W4-4, and 19 of which germinated for Williams82. Samples for DNA preparation were taken by leaf punch, and DNA isolated by citrate extraction. DNA from urea extraction of the 11 proprietary Pioneer varieties was available and used in this validation test, using 2 replicates of each variety. Samples were set up in a 96 well plate, which was aliquoted 4 times into a 384 plate and dried down. For the Taqman assay, each reaction mix is as follows:

DNA (dried down)

| | | |
|---|---|---|
| Water | 2.42 | μl |
| KlearKall Mastermix | 2.5 | μl |
| Forward Primer (100 μM) | 0.0375 | μl |
| Reverse Primer (100 μM) | 0.0375 | μl |
| Probe 1 (100 μM) | 0.005 | μl |
| Probe 2 (100 μM) | 0.005 | μl |
| TOTAL | 5 | μl |

DNA was amplified by PCR using KlearKall (KBioscience) in a hydrocycler using the following conditions:

94° C. 2 min 1 cycle, followed by 40 cycles of:

94° C. 30 sec

60° C. 60 sec

Results of the marker detection reaction were scored and are summarized below.

| | | S12761-1 | S12764-1 | |
|---|---|---|---|---|
| ID | Sample | (ALS1 locus) | (ALS2 locus) | Profile |
| 4518789 | W4-4 | T | G | Als1/als2 |
| 4518791 | W4-4 | T | T | Als1/Als2 |
| 4518792 | W4-4 | T | T | Als1/Als2 |
| 4518794 | W4-4 | T | T | Als1/Als2 |
| 4518796 | W4-4 | T | T | Als1/Als2 |
| 4518797 | W4-4 | T | T | Als1/Als2 |
| 4518798 | W4-4 | T | T | Als1/Als2 |
| 4518799 | W4-4 | T | T | Als1/Als2 |
| 4518801 | W4-4 | T | G | Als1/als2 |
| 4518802 | W4-4 | T | G | Als1/als2 |
| 4518803 | W4-4 | T | T | Als1/Als2 |
| 4518804 | W4-4 | T | T | Als1/Als2 |
| 4518806 | W4-4 | T | T | Als1/Als2 |
| 4518807 | W4-4 | T | T | Als1/Als2 |
| 4518808 | W4-4 | T | T | Als1/Als2 |
| 4518809 | W4-4 | T | T | Als1/Als2 |
| 4518810 | W4-4 | T | G | Als1/als2 |
| 4518811 | W4-4 | T | G | Als1/als2 |
| 4518814 | W4-4 | T | G | Als1/als2 |
| 4518815 | W4-4 | T | T | Als1/Als2 |
| 4518816 | W4-4 | T | G | Als1/als2 |
| 4518817 | W4-4 | T | T | Als1/Als2 |
| 4518818 | W4-4 | T | T | Als1/Als2 |
| 4518819 | W4-4 | T | G | Als1/als2 |
| 4518820 | W4-4 | T | G | Als1/als2 |
| 4518822 | Williams82 | C | G | als1/als2 |
| 4518823 | Williams82 | C | G | als1/als2 |
| 4518824 | Williams82 | C | G | als1/als2 |
| 4518828 | Williams82 | C | G | als1/als2 |
| 4518830 | Williams82 | C | G | als1/als2 |
| 4518833 | Williams82 | C | G | als1/als2 |
| 4518834 | Williams82 | C | G | als1/als2 |
| 4518835 | Williams82 | C | G | als1/als2 |
| 4518836 | Williams82 | C | G | als1/als2 |
| 4518837 | Williams82 | C | G | als1/als2 |
| 4518839 | Williams82 | C | G | als1/als2 |
| 4518840 | Williams82 | C | G | als1/als2 |
| 4518845 | Williams82 | C | G | als1/als2 |
| 4518846 | Williams82 | C | G | als1/als2 |
| 4518848 | Williams82 | C | G | als1/als2 |
| 4518849 | Williams82 | C | G | als1/als2 |
| 4518850 | Williams82 | C | G | als1/als2 |
| 4518851 | Williams82 | C | G | als1/als2 |
| 4518852 | Williams82 | C | G | als1/als2 |
| 4518855 | 95B97 | C | G | als1/als2 |
| 4518856 | 95B97 | C | G | als1/als2 |
| 4518857 | 95M30 | C | G | als1/als2 |
| 4518858 | 95M30 | C | G | als1/als2 |
| 4518859 | 95M50 | T | G | Als1/als2 |
| 4518860 | 95M50 | T | G | Als1/als2 |
| 4518861 | 95M60 | C | G | als1/als2 |
| 4518862 | 95M60 | C | G | als1/als2 |
| 4518863 | 95M82 | C | G | als1/als2 |
| 4518864 | 95M82 | C | G | als1/als2 |
| 4518865 | 96B01 | C | G | als1/als2 |
| 4518867 | 96B01 | C | G | als1/als2 |
| 4518868 | 96B21 | C | G | als1/als2 |
| 4518869 | 96B21 | C | G | als1/als2 |
| 4518870 | 97M50 | C | G | als1/als2 |
| 4518871 | 97M50 | C | G | als1/als2 |
| 4518872 | 98Y30 | C | G | als1/als2 |
| 4518873 | 98Y30 | C | G | als1/als2 |
| 4518874 | 98Y51 | C | G | als1/als2 |
| 4518875 | 98Y51 | C | G | als1/als2 |
| 4518876 | 99R01 | C | G | als1/als2 |
| 4518877 | 99R01 | C | G | als1/als2 |

The marker profiles for this source of W4-4 seed demonstrate that it was likely not a genetically pure source, showing 25/25 with the Als1 tolerance allele, 16/25 with the Als2 tolerance allele and 9/25 with the als2 susceptible allele. We suspect that the sample of W4-4 used in this study was actually a mixture of W20 and W4-4 seed, wherein the mixing of genotypes occurred due to human error. However, these results provide an ideal demonstration of the value of the Als1 and Als2 markers. Without the markers, the impurity of the W4-4 sample would have had to be determined by progeny testing of many single plants and their progeny to confirm which ones were pure for both mutations. Without having any markers to genotype and to select for plants having both the Als1 tolerance allele and the Als2 tolerance allele, genetic purity, segregants or revertants can develop or be maintained within a line, and be very difficult to detect or remove.

All Williams82 samples tested had the expected wt marker profile als1/als2, with susceptible alleles at both ALS loci. The proprietary Pioneer varieties all had the wildtype susceptible marker profile, except for 95M50, which had the Als1 mutant (tolerant) allele. Pioneer variety 95M50 is an STS™ variety developed from a line comprising the Als1 mutation, which is confirmed by this marker profile.

Validated markers were used to genotype another panel of soybean lines, using the methodology essentially as described above. Some of the genotyped varieties had also been phenotypically characterized, as shown in the table below. Results of the marker detection reaction were scored and are summarized below.

| | Genotype | | | | |
|---|---|---|---|---|---|
| | S12761- | | | Phenotype | |
| GE_ID | 1 | S12764-1 | Genotype | Screened | Phenotype |
| 1441020 | T | G | Als1/als2 | No | — |
| 11309493 | T | G | Als1/als2 | No | — |
| 3903279 | T | G | Als1/als2 | No | — |
| 5613712 | T | G | Als1/als2 | No | — |
| 11309609 | T | G | Als1/als2 | No | — |
| 9932081 | T | G | Als1/als2 | No | — |
| 10563663 | T | G | Als1/als2 | No | — |
| 21525771 | T | G | Als1/als2 | No | — |
| 21525772 | T | G | Als1/als2 | No | — |
| 21525767 | C, T | G | Als1(het)/als2 | No | — |
| 13466 | T | G | Als1/als2 | Yes | STS |
| 13503 | T | G | Als1/als2 | Yes | STS |
| 632345 | T | G | Als1/als2 | Yes | STS |
| 1756109 | T | G | Als1/als2 | Yes | STS |
| 5613804 | T | G | Als1/als2 | Yes | STS |
| 12992740 | T | G | Als1/als2 | Yes | STS |
| 7517726 | T | G | Als1/als2 | Yes | STS |
| 10564138 | T | G | Als1/als2 | Yes | STS |
| 24895125 | T | G | Als1/als2 | Yes | STS |
| 632332 | T | G | Als1/als2 | Yes | STS |
| 11309630 | C, T | G | Als1(het)/als2 | Yes | STS |

As before, the markers confirmed expected genotypes based on phenotypic information if available, and identified lines heterozygous for one or more of the alleles. These SNP markers could be useful, for example, for detecting and/or selecting soybean plants with improved tolerance to ALS-inhibiting herbicides. The physical position of each locus and each SNP has been provided. Any marker capable of detecting a polymorphism at one of these physical positions, or a marker closely linked thereto, could also be useful, for example, for detecting and/or selecting soybean plants with improved ALS-inhibiting herbicide tolerance. In some examples, the SNP allele present in the tolerant parental line could be used as a favorable allele to detect or select plants with improved tolerance. In other examples, the SNP allele present in the susceptible parent line could be used as an unfavorable allele to detect or select plants without improved tolerance.

### Example 4

### Evaluation of Herbicide Tolerance for Als1, Als2, and Als1+Als2 Mutants

Soybean lines having wild type als1 & 2 genes, the Als1 mutation, the Als2 mutation, or both the Als1 and Als2 mutations were evaluated for their response to various herbicides. Two seeds from each line were planted into 4-inch pots containing Redi-Earth potting mix (Sun Gro Horticulture Canada Ltd. 52130 RR65, Seba Beach, Alberta TOE 2B0), with two replications planted for each treatment. Pots were placed in flats at 15 pots/flat, and the resulting plants were grown in a greenhouse environment supplemented with lighting (16 hr photoperiod) for the duration of the test. Selected herbicides representing the mode of action that inhibits the acetolactate synthase enzyme, also known as the acetohydroxyacid synthase enzyme, were applied postemergence to soybean when soybean plants were at the V2 growth stage, which is the stage at which the first two trifoliolate leaves were expanded.

Eleven active ingredients representing the five chemical families with herbicidal activity on the acetolactate synthase enzyme were tested. These herbicides can cause a phytotoxic response in wild type soybean plants. The five chemical families tested were sulfonylaminocarbonyltriazolinone (also known as triazolinone), imidazolinone, sulfonylurea, pyrimidinylthiobenzoate, and triazolopyrimidine. Application rates ranged from 0.5 times to eight times the currently labeled application rate for each active ingredient with spray application volume at 30 gallons/acre. The application rates tested were 0.5×, 1×, 2×, 4× and 8× the label rate for each herbicide. Herbicidal active ingredients were applied as formulated materials dispersed in water containing 0.25% nonionic surfactant (Trend® 90).

The test was evaluated by visually scoring plant phenotype 14 days after the herbicides were applied. Controls consisted of soybean plants of the same variety that received no herbicide treatments for each of all the varieties tested. Soybean response was recorded on a zero to 100 visual scale in which zero is no phytotoxic response and 100 is plant death fourteen days after the herbicides were applied. Soybean response (percent phytotoxicity) to postemergence applications of herbicides inhibiting the acetolactate synthase enzyme test results are shown in Table 1.

TABLE 1

| | Active | Rate | Wild | | | Als1 + |
|---|---|---|---|---|---|---|
| Chemical family | ingredient | (g ai/ha) | type | Als1 | Als2 | Als2 |
| Imidazolinone | Imazapyr | 8.75 | 10 | 0 | 5 | 0 |
| Imidazolinone | Imazapyr | 17.5 | 40 | 10 | 20 | 0 |
| Imidazolinone | Imazapyr | 35 | 75 | 45 | 25 | 0 |
| Imidazolinone | Imazapyr | 70 | 90 | 80 | 50 | 40 |
| Imidazolinone | Imazapyr | 140 | 98 | 98 | 55 | 55 |
| Imidazolinone | Imazethapyr | 35 | 0 | 0 | 0 | 0 |
| Imidazolinone | Imazethapyr | 70 | 0 | 0 | 0 | 0 |
| Imidazolinone | Imazethapyr | 140 | 0 | 0 | 0 | 0 |
| Imidazolinone | Imazethapyr | 280 | 15 | 10 | 0 | 5 |
| Imidazolinone | Imazethapyr | 560 | 20 | 20 | 5 | 10 |
| Pyrimidinylthio- | Pyrithiobac | 35 | 70 | 20 | 20 | 0 |
| benzoic acid | sodium | | | | | |
| Pyrimidinylthio- | Pyrithiobac | 70 | 95 | 50 | 30 | 5 |
| benzoic acid | sodium | | | | | |
| Pyrimidinylthio- | Pyrithiobac | 140 | 98 | 70 | 40 | 25 |
| benzoic acid | sodium | | | | | |
| Pyrimidinylthio- | Pyrithiobac | 280 | 98 | 98 | 45 | 45 |
| benzoic acid | sodium | | | | | |
| Pyrimidinylthio- | Pyrithiobac | 560 | 98 | 97 | 49 | 50 |
| benzoic acid | sodium | | | | | |
| Sulfonylurea | Chlorimuron | 8.75 | 0 | 0 | 0 | 0 |
| Sulfonylurea | Chlorimuron | 17.5 | 5 | 0 | 0 | 0 |
| Sulfonylurea | Chlorimuron | 35 | 20 | 0 | 0 | 0 |
| Sulfonylurea | Chlorimuron | 70 | 35 | 0 | 0 | 0 |
| Sulfonylurea | Chlorimuron | 140 | 45 | 0 | 0 | 0 |
| Sulfonylurea | Nicosulfuron | 17.5 | 0 | 0 | 0 | 0 |
| Sulfonylurea | Nicosulfuron | 35 | 10 | 0 | 0 | 0 |
| Sulfonylurea | Nicosulfuron | 70 | 20 | 0 | 0 | 0 |
| Sulfonylurea | Nicosulfuron | 140 | 45 | 5 | 20 | 0 |
| Sulfonylurea | Nicosulfuron | 280 | 65 | 25 | 35 | 5 |
| Sulfonylurea | Rimsulfuron | 8.75 | 10 | 0 | 0 | 0 |
| Sulfonylurea | Rimsulfuron | 17.5 | 35 | 0 | 0 | 0 |
| Sulfonylurea | Rimsulfuron | 35 | 85 | 20 | 20 | 0 |
| Sulfonylurea | Rimsulfuron | 70 | 95 | 40 | 60 | 10 |
| Sulfonylurea | Rimsulfuron | 140 | 98 | 80 | 80 | 10 |
| Sulfonylurea | Sulfometuron | 4.38 | 60 | 0 | 35 | 0 |
| Sulfonylurea | Sulfometuron | 8.75 | 95 | 0 | 40 | 0 |
| Sulfonylurea | Sulfometuron | 17.5 | 98 | 10 | 70 | 0 |
| Sulfonylurea | Sulfometuron | 35 | 98 | 55 | 75 | 10 |
| Sulfonylurea | Sulfometuron | 70 | 98 | 80 | 80 | 5 |
| Sulfonylurea | Thifensulfuron | 4.38 | 0 | 0 | 0 | 0 |
| Sulfonylurea | Thifensulfuron | 8.75 | 10 | 0 | 0 | 0 |
| Sulfonylurea | Thifensulfuron | 17.5 | 10 | 0 | 0 | 0 |
| Sulfonylurea | Thifensulfuron | 35 | 25 | 0 | 0 | 0 |
| Sulfonylurea | Thifensulfuron | 70 | 45 | 0 | 0 | 0 |
| Sulfonylurea | Tribenuron | 4.38 | 10 | 0 | 0 | 0 |
| Sulfonylurea | Tribenuron | 8.75 | 70 | 0 | 15 | 0 |
| Sulfonylurea | Tribenuron | 17.5 | 93 | 0 | 40 | 0 |
| Sulfonylurea | Tribenuron | 35 | 98 | 0 | 60 | 0 |
| Sulfonylurea | Tribenuron | 70 | 98 | 10 | 65 | 0 |
| Triazolinone | Flucarbazone | 17.5 | 60 | 0 | 5 | 0 |
| Triazolinone | Flucarbazone | 35 | 80 | 0 | 15 | 0 |
| Triazolinone | Flucarbazone | 70 | 90 | 5 | 20 | 0 |
| Triazolinone | Flucarbazone | 140 | 98 | 10 | 25 | 0 |
| Triazolinone | Flucarbazone | 280 | 98 | 10 | 30 | 0 |
| Triazolo- | Cloransulam | 8.75 | 0 | 0 | 0 | 0 |
| pyrimidine | methyl | | | | | |
| Triazolo- | Cloransulam | 17.5 | 0 | 0 | 0 | 0 |
| pyrimidine | methyl | | | | | |
| Triazolo- | Cloransulam | 35 | 0 | 0 | 0 | 0 |
| pyrimidine | methyl | | | | | |
| Triazolo- | Cloransulam | 70 | 5 | 0 | 0 | 0 |
| pyrimidine | methyl | | | | | |
| Triazolo- | Cloransulam | 140 | 15 | 0 | 0 | 0 |
| pyrimidine | methyl | | | | | |

The presence of the Als1 gene only or the Als2 gene only substantially improves soybean tolerance to all active ingredients when responses of soybeans containing either the Als1 gene or the Als2 gene are compared to the wild type soybean. The higher doses of both rimsulfuron and sulfometuron cause substantial phytotoxicity to soybean containing only the Als1 gene or the Als2 gene. However, soybean containing both the Als1 and the Als2 genes are extremely tolerant to the higher doses of rimsulfuron and sulfometuron indicating the Als1 gene and the Als2 gene are acting synergistically to improve soybean crop tolerance as confirmed by isobole analysis of the data.

Results in Table 1 were evaluated using the isobole method for analyzing interactions. The isobole method is based on the dose response of the biologically active agents in a combination and uses ‘isoeffective’ or equally effective doses for each of the components to build an isobole graph. Here the biologically active agents that are being assessed for interaction (synergy) are gene mutations in soybean. The effectiveness of the genes in providing improved tolerance to ALS-inhibiting herbicides was assessed by applying increasing doses of herbicides to each soybean line with its mutation(s).

An isobole reduces a dose response surface in three dimensional space to a two dimensional plot by fixing a defined response level (e.g., 50% response). The isobole plot is a graph in the Cartesian plane with a straight line connecting the isoeffective rates of the individual components of the combination. This straight line represents expected responses for zero interactive combinations. The isoeffective rate used to create the isobole is often the EC₅₀, the rate estimated to elicit 50% response, but other ECx values can be used. Since the isobole method is based on the potency of the components of the combination, an experimental design testing a range of rates expected to elicit the full range of responses (e.g., 0 to maximum response) is best. (Ferry et al. (2005) 2005 Conference on Applied Statistics in Agriculture Proceedings pp 33-50, Manhattan, Kans., Kansas State Univ.; Green and Streibig (1993) Herbicide Mixtures, pp 117-134 in Herbicide Bioassays, Streibig and Hudsk Eds, CRC, Boca Raton, Fla.).

Loglogistic dose response curves were fit to percent phytotoxicity response data using log 10 of dose (McCullagh and Nelder (1989) Generalized Linear Models, Chapman & Hall, London). Curves were fit using the logit link with normal errors. Inverse prediction was used to calculate the EC₅₀ or EC₁₀, rate of herbicide estimated to cause 50% or 10% phytotoxic response. Ninety five percent confidence intervals for the ECxs were calculated using Fieller's theorem (Finney (1971) Probit Analysis 3^{rd} Ed., Cambridge University Press, Cambridge). These ECx values and their confidence intervals are inputs for the isobole method.

The isobole shows a ‘zero interactive’ line, the straight line connecting the ECx (e.g. EC50) values for response of the two individual mutants (Als1 and Als2) to the herbicide. The observed response for the combination (Als 1+Als2) is plotted by parsing out the combinations' ECx into its Als1 and Als2 components (using a 1:1 ratio assumption). If the zero interactive line is below the curve generated for Als1+Als2, it indicates a synergistic response, it took more herbicide than expected to generate X % phytotoxicity. Isoeffective curves are drawn connecting the lower confidence intervals and the upper confidence intervals. If the zero interactive line is below the lower confidence interval for the ECx for the observed response of the combination then synergy is indicated. This method is generally valid regardless of shape of dose response curves or mechanism of action.

Isobole analysis was used to assess synergy or antagonism effect of the combination of Als1 and Als2 mutations on soybean tolerance to various herbicides. If full dose response curve was generated (i.e., responses from 20% to 80%), then the EC50 value was used for analysis. In cases where the maximum response was only 10% for any of the mutations for a given herbicide, then the EC10 was used for the isobole analysis in order to not extrapolate or assume that applying more herbicide would cause more response. Herbicides chlorimuron, cloransulam methyl and thifensulfuron did not cause any phytotoxic response for lines having Als1, Als2, or Als1+Als2, therefore dose response analysis could not be performed for these compounds.

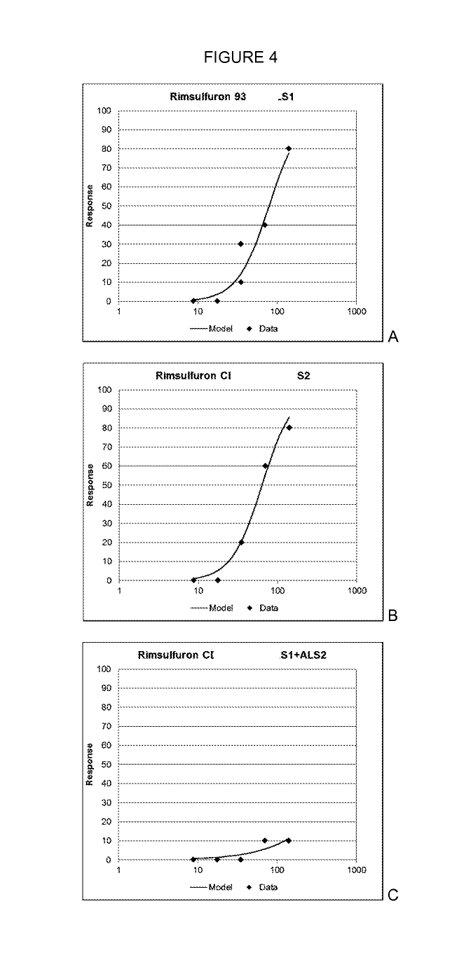

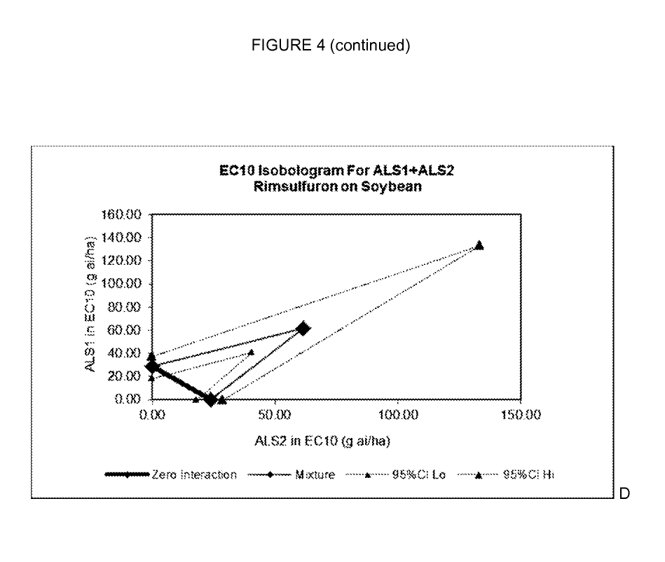

An example of the dose response curves that generated the ECx values and the isobole plot used to assess synergy analysis are shown in FIG. 4, parts A-D. FIG. 4 A shows a dose-response curve for soybean plants with Als1 only treated with rimsulfuron. Similar plots are shown for soybean plants with Als2 alone (FIG. 4 B) or Als1+Als2 (FIG. 4 C), each treated with rimsulfuron as described above. FIG. 4 D shows the isobole plot based on EC10 for Als1+Als2 when treated with rimsulfuron. As seen in the isobole plot, the Als1+Als2 line for observed response and the lower confidence interval are both above the zero interactive line, indicating a stronger protection to the herbicide in plants having both mutations. This can also be stated as the combination of the mutations is providing a safening effect to the herbicide. The observed responses were beyond those expected from additive effects alone, and were determined to be synergistic. Dose-response and isobole analysis data is summarized in Table 2.

TABLE 2

| | | | Avg response (% phytotoxicity) | | |
|---|---|---|---|---|---|
| | Rate | Label | Trait | | |
| Chemical | (g ai/ha) | Rate | Als1 | Als2 | Als1 + Als2 |
| Flucarbazone | 17.5 | 0.5X | 0 | 5 | 0 |
| | 35 | 1X | 0 | 30 | 0 |
| | 70 | 2X | 5 | 50 | 0 |
| | 140 | 4X | 10 | 55 | 0 |
| | 280 | 8X | 10 | 60 | 0 |
| EC10 | | | 230.45 | 8.14 | >280 |
| EC10 (LL, UL*) | | | (118.37, 1829.32) | (0.54, 20.08) | Synergy |
| Isobole Result | | | | | |
| Imazapyr | 8.75 | 0.5X | 0 | 5 | 0 |
| | 17.5 | 1X | 10 | 20 | 0 |
| | 35 | 2X | 45 | 25 | 0 |
| | 70 | 4X | 80 | 50 | 40 |
| | 140 | 8X | 98 | 55 | 55 |
| EC50 | | | 38.86 | 93.91 | 112.81 |
| EC50 (LL, UL*) | | | (34.23, 44.17) | (71.17, 140.56) | (86.7, 172.06) |
| Isobole Result | | | | | Synergy |
| Pyrithiobac | 35 | 0.5X | 20 | 40 | 0 |
| sodium | 70 | 1X | 50 | 60 | 5 |
| | 140 | 2X | 70 | 80 | 25 |
| | 280 | 4X | 98 | 92.5 | 45 |
| | 560 | 8X | 96.5 | 98 | 50 |
| EC50 | | | 74.29 | 49.19 | 448.6 |
| EC50 (LL, UL*) | | | (61.64, 88.58) | (45.83, 52.51) | (331.28, 747.47) |
| Isobole Result | | | | | Synergy |
| Rimsulfuron | 8.75 | 0.5X | 0 | 0 | 0 |
| | 17.5 | 1X | 0 | 0 | 0 |
| | 35 | 2X | 20 | 20 | 0 |
| | 70 | 4X | 40 | 60 | 10 |
| | 140 | 8X | 80 | 80 | 10 |
| EC10 | | | 28.61 | 23.88 | 123.22 |
| EC10 (LL, UL*) | | | (18, 37.16) | (18.09, 28.87) | (81.01, 267.13) |
| Isobole Result | | | | | Synergy |
| Sulfometuron | 4.38 | 0.5X | 0 | 35 | 0 |
| | 8.75 | 1X | 0 | 40 | 0 |
| | 17.5 | 2X | 10 | 70 | 0 |
| | 35 | 4X | 55 | 75 | 10 |
| | 70 | 8X | 80 | 80 | 5 |
| EC10 | | | 14.93 | 0.76 | 114.81 |
| EC10 (LL, UL*) | | | (8.95, 19.34) | (0.1, 1.83) | (16.68, 790.24) |
| Isobole Result | | | | | Synergy |
| Tribenuron | 4.38 | 0.5X | 0 | 0 | 0 |
| | 8.75 | 1X | 0 | 15 | 0 |
| | 17.5 | 2X | 0 | 40 | 0 |
| | 35 | 4X | 0 | 60 | 0 |
| | 70 | 8X | 10 | 65 | 0 |
| EC10 | | | 70 | 4.96 | >70 |
| EC10 (LL, UL*) | | | (70, 70) | (1.85, 8.14) | Synergy |
| Isobole Result | | | | | |
| Imazethapyr | 35 | 0.5X | 0 | 0 | 0 |
| | 70 | 1X | 0 | 0 | 0 |
| | 140 | 2X | 0 | 0 | 0 |
| | 280 | 4X | 10 | 0 | 5 |
| | 560 | 8X | 20 | 10 | 10 |
| EC10 | | | 333.22 | 560.00 | 549.93 |
| EC10 (LL, UL*) | | | (6.49, 451.54) | (559.99, 560.00) | (374.30, 2729.79) |
| Isobole Result | | | | | Possible Synergy |
| Nicosulfuron | 17.5 | 0.5X | 0 | 0 | 0 |
| | 35 | 1X | 0 | 0 | 0 |
| | 70 | 2X | 0 | 0 | 0 |
| | 140 | 4X | 5 | 20 | 0 |
| | 280 | 8X | 25 | 35 | 5 |
| EC10 | | | 187.47 | 105.60 | >280 |
| EC10 (LL, UL*) | | | (91.33, 221.72) | (58.45, 137.57) | Probable Synergy |
| Isobole Result | | | | | |

*LL and UL are the lower limit and upper limit, respectively, for the 95% confidence interval.

Synergistic interaction between Als1 and Als2 was observed for flucarbazon (triazolinone), imazapyr (imidazolinone), pyrithiobac sodium (pyrimidinylthiobenzoate), and rimsulfuron, sulfometuron, and tribenuron (sulfonylureas). For imazethapyr (imidazolinone) and for nicosulfuron (sulfonylurea) the data indicated possible or probable synergy effect respectively. In the analysis of imazethapyr, the entire confidence interval is not above the zero interactive line, but the data suggests possible synergy effect. An EC10 could not be estimated for nicosulfuron Als1+Als2 as the 8× rate only gave 5% phytotoxicity. In order to graph the isobole plot, a conservative EC10 of was used (292), which is greater than the highest rate tested (280). The confidence interval was broad, and given these caveats the conservative conclusion from isobole analysis was probable synergy.

### Example 5

### Evaluation of Herbicide Tolerance for Als1, Als2, and Als1+Als2 Mutants in the Field

Soybean lines having wild type als1 & 2 genes, the Als1 mutation, the Als2 mutation, or both the Als1 and Als2 mutations were evaluated for their response to DUPONT™ Affinity® Broadspec herbicide which contains a combination of sulfonylurea active ingredients, thifensulfuron-methyl (25% by weight) and tribenuron-methyl (25% by weight).

The ALS gene classes, Als1, Als2, and Als1+Als2 were tested at 3 different Iowa field locations using a split-plot design with each location having 6 replications and 4 different treatments: unsprayed check lines, and Affinity® Broadspec applied to the plants at the V3 stage at 1× (17.5 g ai/ha, or 0.5 oz/a), 2×(35 g ai/ha), and 4×(70.0 g ai/ha) application rates. Crop response, % crop damage on a 0-100% scale, was assessed at several time points: 3, 7, and 14 days after treatment (DAT), wherein 0%=no injury, and 100%=crop death. The crop response and yield results are summarized in Table 3.

TABLE 3

| | | Als1 (%) | | Als2 (%) | Als1 + 2 (%) | | |
|---|---|---|---|---|---|---|---|
| Rate | DAT | Variety1 | Variety2 | Variety2 | Variety1 | Variety2 | Variety3 |
| 1X | 3 | 29.5 | 30.3 | 31.9 | 17.0 | 18.9 | 19.8 |
| | 7 | 27.4 | 32.5 | 46.7 | 15.1 | 16.6 | 15.7 |
| | 14 | 14.7 | 19.2 | 53.8 | 7.7 | 8.0 | 7.4 |
| 2X | 3 | 34.6 | 37.6 | 35.9 | 22.0 | 24.9 | 24.7 |
| | 7 | 42.3 | 46.6 | 59.7 | 20.1 | 23.3 | 21.5 |
| | 14 | 33.4 | 39.1 | 68.3 | 13.3 | 13.3 | 11.7 |
| 4X | 3 | 40.8 | 42.7 | 36.7 | 29.0 | 28.5 | 30.3 |
| | 7 | 57.0 | 61.4 | 66.1 | 25.4 | 27.6 | 25.1 |
| | 14 | 61.8 | 67.2 | 79.3 | 16.0 | 19.1 | 16.3 |

The data results in Table 3 for Variety1 and Variety2 were evaluated using the isobole method for analyzing interactions, essentially as described in Example 4 with the following considerations:
1. The data sets for Variety1 and Variety2 were analyzed separately as well as combined. Similar mean and variability for each variety were observed, and the data set for each variety generated the same isobole conclusion.
2. The dataset for each location was analyzed separately because of differences in mean and variation. However, the isobole conclusions were the same for all locations.
3. In at least some instances for Als2 there is an unbalanced experimental design, but ECx was estimable and sufficient data for valid isobole analysis was available.

TABLE 4

| | | | Avg response (% crop response) | | |
|---|---|---|---|---|---|
| | Rate | Label | Trait | | |
| Chemical | (oz/a) | Rate | Als1 | Als2 | Als1 + Als2 |
| 3 DAT | | | | | |
| Location1 | | | | | |
| Tribenuron + | 0.5 | 1X | 26.25 | 30.83 | 12.50 |
| Thifensulfuron | 1 | 2X | 34.17 | 31.67 | 17.92 |
| | 2 | 4X | 34.58 | 28.33 | 24.58 |
| EC25 | | | 0.30 | ~0.4 | 2.07 |
| EC25 | | | (0.04, 0.52) | NA | (1.71, 2.78) |
| (LL, UL*) | | | | | Synergy |
| Isobole Result | | | | | |
| 3 DAT | | | | | |
| Location2 | | | | | |
| Tribenuron + | 0.5 | 1X | 32.92 | 30.83 | 21.25 |
| Thifensulfuron | 1 | 2X | 40.00 | 39.17 | 27.92 |
| | 2 | 4X | 47.92 | 42.50 | 29.17 |
| EC25 | | | 0.21 | 0.20 | 0.83 |
| EC25 | | | (0.12, 0.31) | (0.01, 0.4) | (0.51, 1.12) |
| (LL, UL*) | | | | | Synergy |
| Isobole Result | | | | | |
| 3 DAT | | | | | |
| Location3 | | | | | |
| Tribenuron + | 0.5 | 1X | 30.00 | 34.17 | 20.00 |
| Thifensulfuron | 1 | 2X | 34.58 | 36.67 | 24.17 |
| | 2 | 4X | 42.08 | 39.17 | 32.92 |
| EC25 | | | 0.28 | 0.03 | 0.97 |
| EC25 | | | (0.1, 0.43) | (0, 1.57) | (0.73, 1.2) |
| (LL, UL*) | | | | | Synergy |
| Isobole Result | | | | | |
| 7 DAT | | | | | |
| Location1 | | | | | |
| Tribenuron + | 0.5 | 1X | 31.67 | 42.50 | 19.58 |
| Thifensulfuron | 1 | 2X | 51.25 | 64.17 | 23.33 |
| | 2 | 4X | 65.42 | 70.83 | 29.25 |
| EC25 | | | 0.35 | 0.19 | 1.52 |
| EC25 | | | (0.26, 0.42) | (0.1, 0.27) | (1.08, 3.15) |
| (LL, UL*) | | | | | Synergy |
| Isobole Result | | | | | |
| 7 DAT | | | | | |
| Location2 | | | | | |
| Tribenuron + | 0.5 | 1X | 32.08 | 47.50 | 9.58 |
| Thifensulfuron | 1 | 2X | 47.50 | 57.50 | 19.58 |
| | 2 | 4X | 61.25 | 64.17 | 24.58 |
| EC25 | | | 0.33 | 0.06 | 1.93 |
| EC25 | | | (0.24, 0.41) | (0, 0.16) | (1.63, 2.49) |
| (LL, UL*) | | | | | Synergy |
| Isobole Result | | | | | |
| 7 DAT | | | | | |
| Location3 | | | | | |
| Tribenuron + | 0.5 | 1X | 25.42 | 51.67 | 18.50 |
| Thifensulfuron | 1 | 2X | 34.42 | 55.00 | 21.08 |
| | 2 | 4X | 52.50 | 60.83 | 27.75 |
| EC25 | | | 0.53 | 0.01 | 1.48 |
| EC25 | | | (0.39, 0.65) | (0, 0.07) | (1.07, 2.72) |
| (LL, UL*) | | | | | Synergy |
| Isobole Result | | | | | |
| 14 DAT | | | | | |
| Location1 | | | | | |
| Tribenuron + | 0.5 | 1X | 11.42 | 55.00 | 5.0 |
| Thifensulfuron | 1 | 2X | 31.67 | 70.83 | 7.75 |
| | 2 | 4X | 71.67 | 85.00 | 12.17 |
| EC15 | | | 0.63 | 0.08 | 2.83 |
| EC15 | | | (0.56, 0.7) | (0.05, 0.13) | (1.97, 9.23) |
| (LL, UL*) | | | | | Synergy |
| Isobole Result | | | | | |
| 14 DAT | | | | | |
| Location2 | | | | | |
| Tribenuron + | 0.5 | 1X | 25.42 | 62.50 | 6.25 |
| Thifensulfuron | 1 | 2X | 55.0 | 70.83 | 17.92 |
| | 2 | 4X | 73.33 | 80.83 | 22.50 |
| EC15 | | | 0.30 | 0.02 | 1.03 |
| EC15 | | | (0.23, 0.37) | (0, 0.04) | (0.75, 1.25) |
| (LL, UL*) | | | | | Synergy |
| Isobole Result | | | | | |
| 14 DAT | | | | | |
| Location3 | | | | | |
| Tribenuron + | 0.5 | 1X | 13.83 | 45.83 | 12.50 |
| Thifensulfuron | 1 | 2X | 22.17 | 63.33 | 14.25 |
| | 2 | 4X | 48.75 | 71.67 | 17.17 |
| EC15 | | | 0.64 | 0.07 | 1.15 |
| EC15 | | | (0.5, 0.76) | (0.01, 0.16) | (0.53, 2.65) |
| (LL, UL*) | | | | | Synergy |
| Isobole Result | | | | | |

*LL and UL are the lower limit and upper limit, respectively, for the 95% confidence interval.

In each analysis, except as noted below, the zero interactive line is below the lower confidence interval for the observed response for Als1+Als2, indicating a synergistic response in improving tolerance to tribenuron+thifensulfuron. It took more herbicide than expected to show 25% crop response in the 3 DAT and 7 DAT groups, or 15% crop response in the 14 DAT group. The EC25 could not be estimated from the 3 DAT dose response data for Als2, so the Excel forecast function was used to estimate EC25 between 0 and 0.5. No confidence intervals were calculated for Als2 so all confidence intervals were removed from the isobole. The conclusion of synergy was based on the zero interactive line being below the observed curve for Als1+Als2.

What is claimed is:

1. A method of identifying a soybean plant, germplasm or seed comprising an endogenous polynucleotide encoding a mutant acetolactate synthase gene in its genome, said method comprising isolating nucleic acids from said plant, germplasm or seed, and detecting at least one allele of one or more marker locus that is associated with herbicide resistance, wherein the one or more marker locus is selected from the group consisting of:
(a) S12761-1 on linkage group C1;
(b) a marker locus comprising nucleotides 43645228-43645929 of Glyma04g37270.1;
(c) a marker that detects a polymorphism at nucleotide 43645620 of Glyma04g37270.1;
(d) a marker that detects a polymorphism that encodes a P178S mutation in an acetolactate synthase gene on chromosome 4;
(e) a marker locus closely linked to a marker locus of (a), (b), (c), or (d);
(f) S12764-1 on linkage group C2;
(g) a marker locus comprising nucleotides 14143182-14143881 of Glyma06g17790.1;
(h) a marker that detects a polymorphism at nucleotide 14143499 of Glyma06g17790.1;
(i) a marker that detects a polymorphism that encodes a W560L mutation in an acetolactate synthase gene on chromosome 6; and,
(j) a marker locus closely linked to a marker locus of (f), (g), (h), or (i).

2. The method of claim 1, comprising detecting two or more marker loci of (a)-(j), wherein said two or more marker loci are located on different linkage groups.

3. The method of claim 1, wherein the at least one allele is a favorable allele that positively correlates with improved herbicide resistance when compared to a soybean plant lacking the favorable allele, wherein the at least one favorable allele selected from the group consisting of allele T of S12761-1, allele T of S12764-1, allele Tat nucleotide 43645620 of Glyma04g37270.1, and allele Tat nucleotide 14143499 of Glyma06g17790.1.

4. The method of claim 1, wherein the herbicide comprises an herbicidal compound selected from the group consisting of:
(a) a sulfonylurea;
(b) a pyrimidinylsulfonylurea;
(c) a triazinylsulfonylurea;
(d) a chlorimuron;
(e) a sulfometuron;
(f) a nicosulfuron;
(g) a rimsulfuron;
(h) a thifensulfuron;
(i) a tribenuron;
(j) an imidazolinone;
(k) an imazamethabenz;
(l) an imazamox;
(m) an imazapicl;
(n) an imazapyr;
(o) an imazaquin;
(p) an imazethapyr;
(q) a pyrimidinylthiobenzoate;
(r) a pyrithiobac sodium;
(s) a triazolinone;
(t) a flucarbazone;
(u) a triazolopyrimidine; and,
(v) a cloransulam methyl.

5. A kit for characterizing at least one soybean plant, germplasm or seed, the kit comprising:
(a) primers or probes for detecting one or more marker loci selected from the group consisting of S12761-1, S12764-1, and markers closely linked thereto; and
(b) instructions for using the primers or probes to detect the one or more marker loci and for correlating the detected marker loci with predicted tolerance to at least one herbicide.

6. The kit of claim 5, wherein marker loci S12761-1 and S12764-1 are detected.

7. The kit of claim 5, wherein the primers or probes comprise one or more of SEQ ID NOs: 1-18.

8. An isolated polynucleotide capable of detecting a marker locus selected from the group consisting of:
(a) S12761-1 on linkage group C1;
(b) a marker locus comprising nucleotides 43645228-43645929 of Glyma04g37270.1;
(c) a marker that detects a polymorphism at nucleotide 43645620 of Glyma04g37270.1;
(d) a marker that detects a polymorphism that encodes a P178S mutation in an acetolactate synthase gene on chromosome 4;
(e) S12764-1 on linkage group C2;
(f) a marker locus comprising nucleotides 14143182-14143881 of Glyma06g17790.1;
(g) a marker that detects a polymorphism at nucleotide 14143499 of Glyma06g17790.1;
(h) a marker that detects a polymorphism that encodes a W560L mutation in an acetolactate synthase gene on chromosome 6; and,
(i) a nucleotide sequence selected from the group consisting of SEQ ID NOs: 1-18.

9. An elite soybean plant, germplasm or seed identified by the method of claim 1, said plant, germplasm or seed comprising at least one endogenous polynucleotide encoding an mutant acetolactate synthase gene in its genome, said mutant acetolactate synthase gene encoding a mutation selected from the group consisting of a P178S mutation in an acetolactate synthase gene on chromosome 4, and a W560L mutation in an acetolactate synthase gene on chromosome 6, wherein said plant or germplasm has improved herbicide resistance when compared to a soybean plant or germplasm lacking a mutant acetolactate synthase gene in its genome.

10. The elite soybean plant, germplasm, or seed of claim 9, wherein the plant, germplasm or seed comprises the P178S mutation in an acetolactate synthase gene on chromosome 4 and the W560L mutation in an acetolactate synthase gene on chromosome 6.

11. The elite soybean plant, germplasm or seed of claim 9, wherein the herbicide comprises an herbicidal compound selected from the group consisting of:
(a) a sulfonylurea;
(b) a pyrimidinylsulfonylurea;
(c) a triazinylsulfonylurea;
(d) a chlorimuron;
(e) a sulfometuron;
(f) a nicosulfuron;
(g) a rimsulfuron;
(h) a thifensulfuron;
(i) a tribenuron;
(j) an imidazolinone;
(k) an imazamethabenz;
(l) an imazamox;
(m) an imazapicl;
(n) an imazapyr;
(o) an imazaquin;
(p) an imazethapyr;
(q) a pyrimidinylthiobenzoate;
(r) a pyrithiobac sodium;
(s) a triazolinone;
(t) a flucarbazone;
(u) a triazolopyrimidine; and,
(v) a cloransulam methyl.

12. The elite soybean plant, germplasm or seed of claim 9 further comprising resistance to a herbicidal formulation comprising a compound selected from the group consisting of a hydroxyphenylpyruvatedioxygenase inhibitor, a glyphosate, a sulfonamide, an imidazolinone, a bialaphos, a phosphinothricin, a metribuzin, a mesotrione, an isoxaflutole, an azafenidin, a butafenacil, a sulfosate, a glufosinate, a dicamba, a 2,4-D, and a protox inhibitor.

13. The elite soybean plant, germplasm or seed of claim 12, wherein resistance to the herbicidal formulation is conferred by a transgene.

14. The elite soybean plant, germplasm or seed of claim 9 further comprising a trait selected from the group consisting of drought tolerance, stress tolerance, disease resistance, enhanced yield, modified oil, modified protein, tolerance to chlorotic conditions, and insect resistance.

15. The elite soybean plant, germplasm or seed of claim 14, wherein the trait is selected from the group consisting of brown stem rot resistance, charcoal rot drought complex resistance, Fusarium resistance, Phytophthora resistance, stem canker resistance, sudden death syndrome resistance, Sclerotinia resistance, Cercospora resistance, target spot resistance, frogeye leaf spot resistance, soybean cyst nematode resistance, root knot nematode resistance, rust resistance, high oleic, low linolenic, aphid resistance, stink bug resistance, and iron chlorosis deficiency tolerance.

16. A method for selectively controlling weeds in a field containing a soybean crop comprising:
(a) planting the field with soybean plants, germplasm or seed comprising at least one favorable allele of a marker locus of claim 1, wherein said plants, germplasm or seed have tolerance to an ALS-inhibiting herbicide conferred by said favorable allele; and,
(b) applying an effective amount of the herbicide to the field containing the soybean crop to control the weeds without significantly affecting the crop.

17. The method of claim 16 wherein said plants, germplasm or seed comprise two favorable alleles of a marker locus of claim 1.

18. The method of claim 17 wherein said plants, germplasm or seed comprising two favorable alleles have a synergistic level of resistance to one or more ALS-inhibiting herbicides.

19. The method of claim 18, wherein said plants, germplasm or seed comprising two favorable alleles have a synergistic level of resistance to two or more ALS-inhibiting herbicides.

20. The method of claim 16, wherein the herbicide comprises a herbicidal compound selected from the group consisting of:
(a) a sulfonylurea;
(b) a pyrimidinylsulfonylurea;
(c) a triazinylsulfonylurea;
(d) a chlorimuron;
(e) a sulfometuron;
(f) a nicosulfuron;
(g) a rimsulfuron;
(h) a thifensulfuron;
(i) a tribenuron;
(j) an imidazolinone;
(k) an imazamethabenz;
(l) an imazamox;
(m) an imazapicl;
(n) an imazapyr;
(o) an imazaquin;
(p) an imazethapyr;
(q) a pyrimidinylthiobenzoate;
(r) a pyrithiobac sodium;
(s) a triazolinone;
(t) a flucarbazone;
(u) a triazolopyrimidine; and,
(v) a cloransulam methyl.

21. The method of claim 16, wherein the herbicide is applied as a pre-emergent herbicide, a post-emergent herbicide, or a seed treatment.

22. The method of claim 16, further comprising applying to the crop and weeds in the field a simultaneous or chronologically staggered application of the herbicide and optionally an additional herbicide formulation.

23. The method of claim 22, wherein the additional herbicide formulation is applied and the herbicide formulation contains an active ingredient selected from the group consisting of a hydroxyphenylpyruvatedioxygenase inhibitor, a glyphosate, a sulfonamide, an imidazolinone, a bialaphos, a phosphinothricin, a metribuzin, a mesotrione, an isoxaflutole, an azafenidin, a butafenacil, a sulfosate, a glufosinate, a dicamba, a 2,4-D, and a protox inhibitor, wherein said crop seeds or plants further comprise tolerance to the active ingredient of the additional herbicide formulation.

24. The method of claim 23, wherein tolerance to the active ingredient of the additional herbicide formulation is provided by a transgene which confers the tolerance.

25. A method of producing a soybean crop in a field comprising at least one residual ALS-inhibiting herbicide, said method comprising planting the plant, germplasm or seed of claim 9 in a field having residual ALS-inhibiting herbicide present.


SEQUENCE LISTING (39 sequences)

SEQ ID NO: 1 - DNA, 23 bp, Glycine max
aggatgaggtagttgtgcttcgt

SEQ ID NO: 2 - DNA, 18 bp, Glycine max
cctcgccgacgctttaat

SEQ ID NO: 3 - DNA, 14 bp, Glycine max
caggtctcccgccg

SEQ ID NO: 4 - DNA, 13 bp, Glycine max
caggtcccccgcc

SEQ ID NO: 5 - DNA, 700 bp, Glycine max
ggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcycccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattc

SEQ ID NO: 6 - DNA, 27 bp, Glycine max
caactataagagtggagaatcttcctg

SEQ ID NO: 7 - DNA, 23 bp, Glycine max
tgtttgggaatatctcattctcg

SEQ ID NO: 8 - DNA, 16 bp, Glycine max
ccgatcctcccactga

SEQ ID NO: 9 - DNA, 15 bp, Glycine max
tcctccaactgaacc

SEQ ID NO: 10 - DNA, 700 bp, Glycine max
tgaggttcttgatgagttgacaaatggggatgctattgttagtactggggttgggcagcatcaaatgtgggctgcacagttttacaagtacaagagaccgaggcagtggttgacctcggggggtcttggagccatgggttttggattgcctgcggctattggcgctgctgttgctaaccccggcgctgttgtggttgacattgatggagatggtagtttcatcatgaatgttcaagaattggcaactataagagtggagaatcttcctgttaagatattgttgttgaataatcagcatttgggcatggtggttcagtkggaggatcggttctacaagtccaatagagctcacacctatcttggagacccgtctagcgagaatgagatattcccaaacatgctcaagtttgctgacgcttgtgggataccggcagcgcgtgtgacgaagaaggaagagcttagagcagaaattcagaaaatgttggacacccctggcccctaccttcttgatgtcattgtgccacatcaggagcatgtgttgccgatgattcccagtaatggatccttcaaggatgtgataacggagggagacggtagaacgaggtactgattgcctagatcaaatgttccgtgatgcttggtttgtacaatatatatataagataatgctttcatagttgcaggatttggcctgtggtga

SEQ ID NO: 11 - DNA, 1956 bp, Glycine max
atggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcccccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactga

SEQ ID NO: 12 - protein, 651 aa, Glycine max
MAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVPRRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQWEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 13 - DNA, 1956 bp, Glycine max
atggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtctcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactga

SEQ ID NO: 14 - protein, 651 aa, Glycine max
MAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVSRRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQWEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 15 - DNA, 1971 bp, Glycine max
atggcggccacagcttccagaaccaccccattctcttctccctcttcctcttcacaccccaccttccccaaacgcattgctagatccaccctccctctctctcatcaaaccttcattaaacccaaccacgcgctcaaaatcaaatgttccatctccaaacccccctcgccgccggccaccaccaccactgcagcgccggcaacatcagagcccttcgtgtcgcggttcgcctccgctgaaccgcgcaagggctcggacatccttgtggaggcccttgagcggctgggcgtgactaatgtcttcgcctaccccggcggcgcgtcgatggagatccaccaggctctcacgcgctcctccaccatccgcaacgtcctccctcgccacgagcagggcggcatcttcgccgccgagggctacgcgcgctcctctggcctccccggcgtctgcatcgccacctccggccccggcgccaccaacctcgtcagcggcctcgccgacgccttaatggacagcgtccccgtcgtcgccatcaccggccaggtcccccggcgcatgattggcaccgacgccttccaagaaacccccatcgtcgaagtcaccagatccatcaccaagcacaactacctcatcctcgacgtcgacgacattccccgcatcgtcgccgaagccttcttcgtcgccacctccggccgccccggcccggtcctcatcgacatccccaaagacgtccaacagcaactcgccgtgcctaattgggacgagcccatcaacctccccgggtacctcgccaggcttcccaggccccccaccgaggcccaattagagcacattgtcagactcataaccgaggcccaaaagcccgtcctctacgtcggaggtggcagtttgaattccagtgatgaattgagacgctttgtggaactgactggtattcccgttgctagcactttaatgggccttggaacttatcctattggggatgaatattcccttcaaatgttgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgctgagattggaaagaataagcaggcgcatgtgtcggtttgcgcggatttgaagctggccttgcaggggattaatatgattttggagaaaaaaagagtggggggtggtaagcttgatcttggaggctggagggaagagattaatgtgcagaaacacaagtttccattgggttacaaaacattccaggacgccatttccccgcagcatgctattgaggttcttgatgagttgacaaatggggatgctattgttagtactggggttgggcagcatcaaatgtgggctgcacagttttacaagtacaagagaccgaggcagtggttgacctcggggggtcttggagccatgggttttggattgcctgcggctattggcgctgctgttgctaaccccggcgctgttgtggttgacattgatggagatggtagtttcatcatgaatgttcaagaattggcaactataagagtggagaatcttcctgttaagatattgttgttgaataatcagcatttgggcatggtggttcagtgggaggatcggttctacaagtccaatagagctcacacctatcttggagacccgtctagcgagaatgagatattcccaaacatgctcaagtttgctgacgcttgtgggataccggcagcgcgtgtgacgaagaaggaagagcttagagcagaaattcagaaaatgttggacacccctggcccctaccttcttgatgtcattgtgccacatcaggagcatgtgttgccgatgattcccagtaatggatccttcaaggatgtgataacggagggagacggtagaacgaggtactga

SEQ ID NO: 16 - protein, 656 aa, Glycine max
MAATASRTTPFSSPSSSSHPTFPKRIARSTLPLSHQTFIKPNHALKIKCSISKPPSPPATTTTAAPATSEPFVSRFASAEPRKGSDILVEALERLGVTNVFAYPGGASMEIHQALTRSSTIRNVLPRHEQGGIFAAEGYARSSGLPGVCIATSGPGATNLVSGLADALMDSVPVVAITGQVPRRMIGTDAFQETPIVEVTRSITKHNYLILDVDDIPRIVAEAFFVATSGRPGPVLIDIPKDVQQQLAVPNWDEPINLPGYLARLPRPPTEAQLEHIVRLITEAQKPVLYVGGGSLNSSDELRRFVELTGIPVASTLMGLGTYPIGDEYSLQMLGMHGTVYANYAVDNSDLLLAFGVRFDDRVTGKLEAFASRAKIVHIDIDSAEIGKNKQAHVSVCADLKLALQGINMILEKKRVGGGKLDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQWEDRFYKSNRAHTYLGDPSSENEIFPNMLKFADACGIPAARVTKKEELRAEIQKMLDTPGPYLLDVIVPHQEHVLPMIPSNGSFKDVITEGDGRTRY

SEQ ID NO: 17 - DNA, 1971 bp, Glycine max
atggcggccacagcttccagaaccaccccattctcttctccctcttcctcttcacaccccaccttccccaaacgcattgctagatccaccctccctctctctcatcaaaccttcattaaacccaaccacgcgctcaaaatcaaatgttccatctccaaacccccctcgccgccggccaccaccaccactgcagcgccggcaacatcagagcccttcgtgtcgcggttcgcctccgctgaaccgcgcaagggctcggacatccttgtggaggcccttgagcggctgggcgtgactaatgtcttcgcctaccccggcggcgcgtcgatggagatccaccaggctctcacgcgctcctccaccatccgcaacgtcctccctcgccacgagcagggcggcatcttcgccgccgagggctacgcgcgctcctctggcctccccggcgtctgcatcgccacctccggccccggcgccaccaacctcgtcagcggcctcgccgacgccttaatggacagcgtccccgtcgtcgccatcaccggccaggtcccccggcgcatgattggcaccgacgccttccaagaaacccccatcgtcgaagtcaccagatccatcaccaagcacaactacctcatcctcgacgtcgacgacattccccgcatcgtcgccgaagccttcttcgtcgccacctccggccgccccggcccggtcctcatcgacatccccaaagacgtccaacagcaactcgccgtgcctaattgggacgagcccatcaacctccccgggtacctcgccaggcttcccaggccccccaccgaggcccaattagagcacattgtcagactcataaccgaggcccaaaagcccgtcctctacgtcggaggtggcagtttgaattccagtgatgaattgagacgctttgtggaactgactggtattcccgttgctagcactttaatgggccttggaacttatcctattggggatgaatattcccttcaaatgttgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgctgagattggaaagaataagcaggcgcatgtgtcggtttgcgcggatttgaagctggccttgcaggggattaatatgattttggagaaaaaaagagtggggggtggtaagcttgatcttggaggctggagggaagagattaatgtgcagaaacacaagtttccattgggttacaaaacattccaggacgccatttccccgcagcatgctattgaggttcttgatgagttgacaaatggggatgctattgttagtactggggttgggcagcatcaaatgtgggctgcacagttttacaagtacaagagaccgaggcagtggttgacctcggggggtcttggagccatgggttttggattgcctgcggctattggcgctgctgttgctaaccccggcgctgttgtggttgacattgatggagatggtagtttcatcatgaatgttcaagaattggcaactataagagtggagaatcttcctgttaagatattgttgttgaataatcagcatttgggcatggtggttcagttggaggatcggttctacaagtccaatagagctcacacctatcttggagacccgtctagcgagaatgagatattcccaaacatgctcaagtttgctgacgcttgtgggataccggcagcgcgtgtgacgaagaaggaagagcttagagcagaaattcagaaaatgttggacacccctggcccctaccttcttgatgtcattgtgccacatcaggagcatgtgttgccgatgattcccagtaatggatccttcaaggatgtgataacggagggagacggtagaacgaggtactga

SEQ ID NO: 18 - protein, 656 aa, Glycine max
MAATASRTTPFSSPSSSSHPTFPKRIARSTLPLSHQTFIKPNHALKIKCSISKPPSPPATTTTAAPATSEPFVSRFASAEPRKGSDILVEALERLGVTNVFAYPGGASMEIHQALTRSSTIRNVLPRHEQGGIFAAEGYARSSGLPGVCIATSGPGATNLVSGLADALMDSVPVVAITGQVPRRMIGTDAFQETPIVEVTRSITKHNYLILDVDDIPRIVAEAFFVATSGRPGPVLIDIPKDVQQQLAVPNWDEPINLPGYLARLPRPPTEAQLEHIVRLITEAQKPVLYVGGGSLNSSDELRRFVELTGIPVASTLMGLGTYPIGDEYSLQMLGMHGTVYANYAVDNSDLLLAFGVRFDDRVTGKLEAFASRAKIVHIDIDSAEIGKNKQAHVSVCADLKLALQGINMILEKKRVGGGKLDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQLEDRFYKSNRAHTYLGDPSSENEIFPNMLKFADACGIPAARVTKKEELRAEIQKMLDTPGPYLLDVIVPHQEHVLPMIPSNGSFKDVITEGDGRTRY

SEQ ID NO: 19 - protein, 651 aa, Glycine max
MAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVARRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQLEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 20 - DNA, 18 bp, Glycine max
gaaactctccaccgcctc

SEQ ID NO: 21 - DNA, 24 bp, Glycine max
gatcactaagtaaccattaaagac

SEQ ID NO: 22 - DNA, 26 bp, Glycine max
ttaataaattttctacatcccagtga

SEQ ID NO: 23 - DNA, 21 bp, Glycine max
gatgctactgcatgtagtaag

SEQ ID NO: 24 - DNA, 19 bp, Glycine max
gacacactctgagagtctc

SEQ ID NO: 25 - DNA, 23 bp, Glycine max
taccaaaactactgcaaactatg

SEQ ID NO: 26 - DNA, 26 bp, Glycine max
acctaagttaattcatgaaatgtttg

SEQ ID NO: 27 - DNA, 29 bp, Glycine max
gctatattagcttactatttttacaaaac

SEQ ID NO: 28 - DNA, 22 bp, Glycine max
gatcattaaacgttttaacgcg

SEQ ID NO: 29 - DNA, 24 bp, Glycine max
tatcttagttgccaacatgaatac

SEQ ID NO: 30 - DNA, 20 bp, Glycine max
tggtgctacccacacaacac

SEQ ID NO: 31 - DNA, 20 bp, Glycine max
cagtgcagccacacaaagac

SEQ ID NO: 32 - DNA, 18 bp, Glycine max
ctcaccacaggccaaatc

SEQ ID NO: 33 - DNA, 30 bp, Glycine max
catccttgaaggatccattactgggaatca

SEQ ID NO: 34 - DNA, 17 bp, Glycine max
cttcaccaaggaagcgc

SEQ ID NO: 35 - DNA, 15 bp, Glycine max
ttcggcggcgaagac

SEQ ID NO: 36 - DNA, 15 bp, Glycine max
cgccggcaacatcag

SEQ ID NO: 37 - DNA, 15 bp, Glycine max
tcggcggcgaagatg

SEQ ID NO: 38 - DNA, 20 bp, Glycine max
atgctgcgcagacagtcact

SEQ ID NO: 39 - DNA, 24 bp, Glycine max
cagcctcatccaatacaaacatct